(19) United States
(12) Patent Application Publication
(10) Pub. No.: US 2024/0261411 A1
(43) Pub. Date: Aug. 8, 2024

(54) PERMEATION ENHANCERS FOR GASTROINTESTINAL SYNTHETIC EPITHELIAL LININGS

(71) Applicant: Syntis Bio, Inc., Boston, MA (US)

(72) Inventors: Rahul K. DHANDA, Boston, MA (US); Anthony C. YU, San Francisco, CA (US); Maria KANELLI, Cambridge, MA (US); Thomas VON ERLACH, Cambridge, MA (US); James L. WRIGHT, Lexington, MA (US); Edgardo RIVERA-DELGADO, Cambridge, MA (US); Luke C. SEPICH, Somerville, MA (US)

(21) Appl. No.: 18/418,090

(22) Filed: Jan. 19, 2024

Related U.S. Application Data
(60) Provisional application No. 63/541,238, filed on Sep. 28, 2023.
(60) Provisional application No. 63/480,921, filed on Jan. 20, 2023.

(51) Int. Cl. A61K 47/28; A61K 9/00; A61K 38/26; A61K 47/02; A61K 47/12; A61K 47/14; A61K 47/18; A61K 47/22; A61K 47/24; A61K 47/26

(52) U.S. Cl. CPC A61K 47/28; A61K 38/26; A61K 47/02; A61K 47/12; A61K 47/14; A61K 47/183; A61K 47/186; A61K 47/22; A61K 47/24; A61K 47/26; A61K 9/0053

(57) ABSTRACT

The disclosure relates to synthetic linings for the gastrointestinal (GI) tract, “Gastrointestinal Synthetic Epithelial Linings” or “GSELs,” which can modulate absorption of drugs, nutrients, and other substances in the GI tract, and one or more permeation enhancers to aid in such absorption. A GSEL is a temporary lining on the inner surface of the lumen of the GI tract, keeping its active agent (e.g., drug, nutrients, or other substances) in intimate contact with the GI mucosa such that the active agent can be absorbed over long periods of time.

### CROSS-REFERENCE TO RELATED APPLICATIONS

This application claims priority to U.S. provisional application 63/480,921, filed on Jan. 20, 2023, and U.S. provisional application 63/541,238, filed on Sep. 28, 2023, the contents of each is incorporated herein by reference in their entirety.

### FIELD OF THE INVENTION

The invention relates to synthetic linings for the gastrointestinal (GI) tract, which can modulate absorption of drugs, nutrients, and other substances in the GI tract, and permeation enhancers to aid in such absorption.

### BACKGROUND OF THE INVENTION

The gastrointestinal (GI) tract in humans, vertebrates, and other animals serves as a highly versatile organ system with multiple functions. A primary function of the GI tract is absorption of substances, such as nutrients from food. Due to the convenience of oral administration of drugs versus other methods such as injection or inhalation, absorption of substances in the GI tract is also widely utilized for administration of pharmaceuticals. Multiple diverse modes are available for oral administration of drugs, including both immediate release and sustained release formulations.

Many pharmaceuticals cannot be administered orally, however, due to the function of the epithelial lining as a barrier against certain types of substances. Various physical and chemical properties of certain pharmaceuticals prevent passage across the epithelium lining the GI tract. Pharmaceuticals, especially biologics, may be too large for transport across the epithelium. Other properties which affect transport include charge and hydrophobicity.

The current disclosure describes composition, methods, and kits for modulating absorption of pharmaceuticals in the gastrointestinal tract, which can enhance absorption of drugs in the GI tract by prolonging their contact with the gastric mucosa. The current disclosure also provides gastrointestinal synthetic epithelial linings (GSELs) in combination with one or more permeation enhancers, where the permeation enhancers promote absorption of pharmaceuticals in the gastrointestinal tract.

### SUMMARY OF THE INVENTION

The present disclosure relates to synthetic linings formed in situ in the gastrointestinal tract, such as in the small intestine, may be used in combination with an agent and one or more permeation enhancers, where the one or more permeation enhancers increase the absorption of the agent across the intestinal tissue as compared to the synthetic lining without the permeation enhancer. The synthetic lining itself is referred to as a Gastrointestinal Synthetic Epithelial Lining (GSEL).

Disclosed herein is a composition for oral administration for forming a polymer in situ in a subject, comprising: a polymer precursor; an oxygen source; and a permeation enhancer that enhances permeation of one or more active pharmaceutical ingredients. In some embodiments, the composition comprises one or more active pharmaceutical ingredients. In some embodiments, the composition further comprises a buffering agent. In some embodiments, the composition further comprises one or more additional permeation enhancers. In some embodiments, the composition comprises an amount of polymer precursor to (polymer precursor, oxygen source, and permeation enhancer) of 40% to 90%. In some embodiments, the composition comprises an amount of oxygen source to (polymer precursor, oxygen source, and permeation enhancer) of 1% to 15%. In some embodiments, the composition comprises an amount of permeation enhancer to (polymer precursor, oxygen source, and permeation enhancer) of 0.1% to 60%. In some embodiments, the composition comprises an amount of polymer precursor to (polymer precursor, oxygen source, permeation enhancer, and active pharmaceutical ingredient(s)) of 40% to 90%. In some embodiments, the composition comprises an amount of oxygen source to (polymer precursor, oxygen source, permeation enhancer, and active pharmaceutical ingredient(s)) of 1% to 15%. In some embodiments, the composition comprises an amount of permeation enhancer to (polymer precursor, oxygen source, permeation enhancer, and active pharmaceutical ingredient(s)) of 1% to 60%. In some embodiments, the composition comprises an amount of active pharmaceutical ingredient(s) to (polymer precursor, oxygen source, permeation enhancer, and active pharmaceutical ingredient(s)) of 0.1% to 10%.

In some embodiments, the active pharmaceutical ingredient is a macromolecule having a molecular weight between about 1 kD and about 160 kD. In some embodiments, the macromolecule is a polypeptide or polynucleotide. In some embodiments, the macromolecule is a polypeptide. In some embodiments, the polypeptide has a molecular weight of between about 1 kD and about 10 kD. In some embodiments, the polypeptide comprises between about 8 and about 80 amino acids. In some embodiments, the polypeptide comprises insulin, semaglutide, a GLP-1 receptor agonist, tirzepatide, liraglutide, desmopressin, octreotide, an analgesic peptide, difelikefalin, H-20, an antibiotic, cyclosporin, vancomycin, lactase, beta galactosidase, exenatide, teriparatide, nafarelin, buserelin, captopril, daptomycin, an antibody, caplacizumab, ozoralizumab, brolucizumab, ranibizumab, bevacizumab, trastuzumab, rituximab, adalimumab, an enzyme, lipase, a protease, phenylalanine hydroxylase, carbamoylphosphate synthetase I, glucose oxidase, or L-asparaginase. In some embodiments, the macromolecule is a polynucleotide. In some embodiments, the polynucleotide has a molecular weight of between about 5 kD and about 1500 kD. In some embodiments, the polynucleotide is single-stranded. In some embodiments, the polynucleotide is double-stranded. In some embodiments, the polynucleotide is single-stranded and comprises between about 10 and about 5000 bases. In some embodiments, the polynucleotide is single-stranded and comprises between about 10 and about 1000 bases. In some embodiments, the polynucleotide is single-stranded and comprises between about 10 and about 30 bases. In some embodiments, the polynucleotide is double-stranded and comprises between about 5 and about 2500 base pairs. In some embodiments, the polynucleotide is double-stranded and comprises between about 5 and about 500 base pairs. In some embodiments, the polynucleotide is double-stranded and comprises between about 5 and about 15 base pairs. In some embodiments, the polynucleotide comprises an antisense oligonucleotide, mipomersen, patisiran, exondys, an siRNA, or an inflammatory bowel disease (IBD) targeting siRNA. In some embodiments, the active pharmaceutical ingredient is a small molecule having a molecular weight of 1 kD or less.

In some embodiments, the polymer precursor comprises one or both of a monomer and an oligomer precursor to a polymer. In some embodiments, the polymer precursor is selected from Table 1 or Table 2, or combinations thereof. In some embodiments, the monomer is dopamine, dopamine HCl, levodopa, norepinephrine, methyldopa, levodopa methyl ester, levodopa ethyl ester, or combinations thereof. In some embodiments, the oxygen source is a substrate for an endogenous catalyst. In some embodiments, the oxygen source is urea hydrogen peroxide or hydrogen peroxide. In some embodiments, the composition is in an oral dosage form. In some embodiments, the oral dosage form is a solution, a gel, a tablet, a powder, or a capsule. In some embodiments, the oral dosage form comprises one or more solutions, gels, tablets, powders, or capsules. In some embodiments, the oral dosage form is an enteric dosage form.

In some embodiments, the permeation enhancer is a carnitine. In some embodiments, the carnitine is an acyl carnitine. In some embodiments, the carnitine is selected from the group consisting of lauroylcarnitine, palmitoylcarnitine, and palmitoyl carnitine chloride (PCC). In some embodiments, the carnitine is lauroylcarnitine or palmitoylcarnitine. In some embodiments, the permeation enhancer is a choline. In some embodiments, the choline is lysophosphatidyl choline. In some embodiments, the permeation enhancer is an aromatic alcohol. In some embodiments, the aromatic alcohol is selected from the group consisting of propyl gallate, butylated hydroxytoluene, and butylated hydroxyanisole. In some embodiments, the aromatic alcohol is benzyl alcohol, phenyl alcohol, or phenoxyethanol. In some embodiments, the permeation enhancer is a piperazine derivative. In some embodiments, the piperazine derivative is selected from the group consisting of 1-phenylpiperazinc, 1-methyl-4-phenylpiperazine, 1-(4-methylphenyl)piperazine, and 1-benzylpiperazine. In some embodiments, the piperazine derivative is 1-phenylpiperazine or 1-methyl-4-piperazine. In some embodiments, the permeation enhancer is a mucoadhesive polymer. In some embodiments, the mucoadhesive polymer is selected from the group consisting of chitosan, chitosan hydrochloride, trimethylated chitosan chloride, and N,N,N-trimethyl chitosan chloride. In some embodiments, the mucoadhesive polymer is trimethylated chitosan chloride. In some embodiments, the permeation enhancer is a cell penetrating peptide. In some embodiments, the cell penetrating peptide is selected from the group consisting of transportan and penetratin. In some embodiments, the cell penetrating peptide is selected from the group consisting of oligoarginines, polyarginines, oligolysines, polylysines, oligotryptophans, and polytryptophan. In some embodiments, the permeation enhancer is an amino acid. In some embodiments, the amino acid is tryptophan. In some embodiments, the permeation enhancer is an ionic liquid. In some embodiments, the ionic liquid is selected from the group consisting of choline geranate, nicotinic acid and trigonelline. In some embodiments, the ionic liquid is choline geranate. In some embodiments, the permeation enhancer is an organic solvent. In some embodiments, the solvent is selected from the group consisting of ethanol, 2-propanol, 1-propanol, and 2-methyl-2-propanol. In some embodiments, the organic solvent is selected from the group consisting of dimethyl sulfoxide, ethyl acetate, and acetone. In some embodiments, the permeation enhancer is an anionic surfactant. In some embodiments, the anionic surfactant is sodium dodecyl sulphate, or an alternative pharmaceutically acceptable salt thereof. In some embodiments, the anionic surfactant is sodium cholate, or an alternative pharmaceutically acceptable salt thereof. In some embodiments, the permeation enhancer is a chelating agent. In some embodiments, the chelating agent is selected from the group consisting of EDTA, EGTA, and DTPA. In some embodiments, the chelating agent is EDTA. In some embodiments, the permeation enhancer is a non-ionic surfactant. In some embodiments, the non-ionic surfactant is an ethoxylate. In some embodiments, the non-ionic surfactant is an alcohol ethoxylate (CXEY, where X is the alcohol carbon number and Y is the ethylene oxide number). In some embodiments, the non-ionic surfactant is a medium or long chain fatty acid sugar ester. In some embodiments, the non-ionic surfactant is a medium or long chain fatty acid sucrose ester. In some embodiments, the non-ionic surfactant is an ethoxylated fatty acid sugar ester. In some embodiments, the non-ionic surfactant is an ethoxylated sorbitan ester. In some embodiments, the non-ionic surfactant is an ethoxylated glyceride. In some embodiments, the non-ionic surfactant is selected from the group consisting of macrogol-8 glyceride, sucrose esters, sucrose laurate, ethoxylates, alkyl maltosides, dodecyl maltoside, short chain polyethylene glycol, Brij® series, polyoxyethylene(10) oleyl ether, polyoxyethylene (23) lauryl ether, polysorbates, polysorbate series PS 20, PS 40, PS 60, PS 65, PS 80, and Triton X-100. In some embodiments, the non-ionic surfactant is caprylocaproyl polyoxyl-8 glyceride (LABRASOL®), poloxamers, polyoxylglycerides, or polyethylene monostearate. In some embodiments, the permeation enhancer is a non-ionic detergent. In some embodiments, the non-ionic detergent is sucrose monolaurate or n-tetradecyl β-D-maltopyranoside (TDM). In some embodiments, the non-ionic detergent is sucrose monolaurate. In some embodiments, the non-ionic detergent is n-tetradecyl ß-D-maltopyranoside (TDM). In some embodiments, the permeation enhancer is a fatty acid, a fatty acid salt, an ethoxylated fatty acid ester, a sugar fatty acid ester, or an ethoxylated sugar fatty acid ester. In some embodiments, the fatty acid salt is sodium caprate (C₁₀), or an alternative pharmaceutically acceptable salt thereof. In some embodiments, the fatty acid salt is sodium caprylate (C₈), or an alternative pharmaceutically acceptable salt thereof, or sodium laurate (C₁₂), or alternative pharmaceutically acceptable salt thereof. In some embodiments, the sugar fatty acid ester is a fatty acid ester of a monosaccharide. In some embodiments, the sugar fatty acid ester is an ethoxylated fatty acid ester of a monosaccharide. In some embodiments, the sugar fatty acid ester is a fatty acid ester of sorbitan or glucose. In some embodiments, the sugar fatty acid ester is an ethoxylated fatty acid ester of sorbitan or glucose. In some embodiments, the sugar fatty acid ester is a fatty acid ester of a disaccharide. In some embodiments, the sugar fatty acid ester is an ethoxylated fatty acid ester of a disaccharide. In some embodiments, the sugar fatty acid ester is a fatty acid ester of sucrose or maltose. In some embodiments, the sugar fatty acid ester is an ethoxylated fatty acid ester of sucrose or maltose. In some embodiments, the fatty acid, ethoxylated fatty acid ester, sugar fatty acid ester, or ethoxylated sugar fatty acid ester is selected from the group consisting of dodecylmaltoside, sodium dodecyl sulfate, nonaethylene glycol monododecyl ether (C₁₂E₉), sodium laurate (C₁₂), sodium nonanoate (C₉), sodium undecanoate (CH), sodium undecylenate (C11:1), sodium oleate, linoleic acid, and sucrose monolaurate, and pharmaceutically acceptable salts thereof or alternative pharmaceutically acceptable salts thereof. In some embodiments, the fatty acid, ethoxylated fatty acid ester, sugar fatty acid ester, or ethoxylated sugar fatty acid ester is nonaethylene glycol monododecyl ether (C₁₂E₉). In some embodiments, the permeation enhancer is an endogenous secretion. In some embodiments, the endogenous secretion is a bile salt. In some embodiments, the bile salt is selected from the group consisting of sodium taurodeoxycholate, sodium taurocholate, sodium cholate, sodium deoxycholate, sodium glycodeoxycholate, sodium glycochenodeoxycholate, sodium glycocholiate, sodium chenodeoxycholate, sodium taurochenodeoxycholate, sodium lithocholate, and mixed sodium taurodihydrofusidate, or an alternative pharmaceutically acceptable salt thereof. In some embodiments, the bile salt is sodium cholate, or an alternative pharmaceutically acceptable salt thereof. In some embodiments, the permeation enhancer is an N-acylated acid. In some embodiments, the N-acylated acid is acetylsalicylic acid, or a pharmaceutically acceptable salt thereof. In some embodiments, the N-acylated acid is sodium N-[8-(2-hydroxybenzoyl)amino]caprylate (SNAC), or an alternative pharmaceutically acceptable salt thereof, or the corresponding acid thereof. In some embodiments, the N-acylated acid is 8-(N-2-hydroxy-5-chloro-benzoyl)-amino-caprylic acid (5-CNAC), or an alternative pharmaceutically acceptable salt thereof, or the corresponding acid thereof. In some embodiments, the N-acylated acid is 4-[(4-chloro-2-hydroxy-benzoyl)amino]butanoic acid (4-CNAB), or an alternative pharmaceutically acceptable salt thereof, or the corresponding acid thereof. In some embodiments, the N-acylated acid is N-(10-[2-hydroxybenzoyl]-amino)decanoic acid (SNAD), or an alternative pharmaceutically acceptable salt thereof, or the corresponding acid thereof. In some embodiments, the N-acylated acid is monosodium N-(4-chlorosalicyloyl)-4-aminobutyrate (5-CNAB), or an alternative pharmaceutically acceptable salt thereof, or the corresponding acid thereof. In some embodiments, the N-acylated acid is N-[8-(2-hydroxy-4-methoxy)benzoyl]amino caprylic acid (4-MOAC), or a pharmaceutically acceptable salt thereof. In some embodiments, the N-acylated acid is sodium N-[8-(2-hydroxybenzoyl)amino]caprylate (SNAC), or an alternative pharmaceutically acceptable salt thereof, or the corresponding acid thereof. In some embodiments, the permeation enhancer is a high molecular weight polymer. In some embodiments, the high molecular weight polymer is a polysaccharide. In some embodiments, the high molecular weight polymer is an antibacterial toxin. In some embodiments, the antibacterial toxin is selected from the group consisting of zonula occludens toxin analogs, viral protein 8 analogs, and Clostridium perfringens enterotoxin analogs. In some embodiments, the high molecular weight polymer is chitosan or carboxymethylcellulose. In some embodiments, the permeation enhancer is a caprylocaproyl PEG 8 glyceride. In some embodiments, the permeation enhancer is a sugar-based surfactant. In some embodiments, the sugar-based surfactant is dodecyl-ß-D-maltopyranoside (DDM). In some embodiments, the permeation enhancer is glyceryl monocaprate. In some embodiments, the permeation enhancer is urea. In some embodiments, the permeation enhancer is sodium docusate, or an alternative pharmaceutically acceptable salt thereof, or the corresponding acid thereof. In some embodiments, the permeation enhancer is citric acid, or a pharmaceutically acceptable salt thereof. In some embodiments, the permeation enhancer enhances paracellular transport. In some embodiments, the permeation enhancer enhances transcellular transport. In some embodiments, the permeation enhancer enhances paracellular transport and transcellular transport.

Disclosed herein is a method of forming a polymer coating in the small intestine of a subject, the method comprising administering to a subject any one of the compositions disclosed herein.

Disclosed herein is a method of forming a polymer coating in the small intestine of a subject, the method comprising orally administering to a subject: a polymer precursor; an oxygen source; and a permeation enhancer that enhances uptake of one or more active pharmaceutical ingredients; wherein the polymer precursor and the oxygen source contact a catalyst endogenous to the subject and the catalyst polymerizes the polymer precursor. In some embodiments, the method further comprises administering an active pharmaceutical ingredient to the subject. In some embodiments, the polymer precursor, the oxygen source and the permeation enhancer are administered as a single composition. In some embodiments, the polymer precursor, the oxygen source, the permeation enhancer, and the active pharmaceutical ingredient are administered as a single composition. In some embodiments, the polypeptide comprises semaglutide. In some embodiments, the polypeptide comprises tirzepatide.

### BRIEF DESCRIPTION OF THE DRAWINGS

FIG. 1A shows the permeability (expressed as fold change) of the active pharmaceutical ingredient (API) semaglutide delivered using GSEL. Each data point represents the average permeability of a separate formulation. The short horizontal bar represents the mean. Data is normalized to the permeability of GSEL_40 and sorted by the mean permeability. The x-axis is labeled with the excipient abbreviation and concentration in mg/ml (e.g., CHA_80 indicates 80 mg/ml of CHA; CHA indicates Cholic Acid Sodium Salt). All formulations contained GSEL_40 (9.8 mg/mL dopamine HCl, 25 mg/mL PDA, 0.67 mg/mL hydrogen peroxide, 2.2 mg/mL Tris HCl, 4.3 mg/mL Tris base). Table E1 in Example 1 provides a key to the abbreviations used for the excipients.

FIG. 1B shows the percent mean permeability of active pharmaceutical ingredient (API) tirzepatide. Pm=(amount that crossed tissue) divided by (amount initially placed in the upper chamber), multiplied by 100. CV represents the coefficient of variation and is defined as: (standard deviation) divided by (mean), multiplied by 100. Each data point represents the average permeability of a separate formulation. The short horizontal bar represents the mean. Data is sorted by the mean permeability. The x-axis is labeled with the excipient abbreviation and concentration in mg/ml (e.g., CHA_80 indicates 80 mg/ml of CHA; CHA indicates Cholic Acid Sodium Salt). All formulations contained GSEL_40 (9.8 mg/mL dopamine HCl, 25 mg/ml PDA, 0.67 mg/mL hydrogen peroxide, 2.2 mg/mL Tris HCl, 4.3 mg/mL Tris base; values can vary by plus or minus 5%; and at pH 8.5). Table E1 in Example 1 provides a key to the abbreviations used for the excipients, and Table E2 reports the percent permeability (% Pm).

FIG. 1C shows the summary of values determined for each GSEL-single excipient combination. Active pharmaceutical ingredient (API) transported is semaglutide. Fold change GSEL (FC GSEL) measures the fold change of permeability of each formulation with excipient, and normalized by the permeability of GSEL only. Data is sorted by the mean permeability. The numerical value indicates excipient concentration (e.g., GSEL-GCA_40-40 indicates 40 mg/ml GSEL and 40 mg/ml sodium glycocholate). Pm=(amount that crossed tissue) divided by (amount initially placed in the upper chamber), multiplied by 100. CV represents the coefficient of variation and is defined as: (standard deviation) divided by (mean), multiplied by 100.

FIG. 1D shows the summary of values determined for each GSEL-single excipient combination. Active pharmaceutical ingredient (API) transported is tirzepatide. Fold change GSEL (FC GSEL) measures the fold change of permeability of each formulation with excipient, and normalized by the permeability of GSEL only. Data is sorted by the mean permeability. The value indicates excipient concentration. Pm=(amount that crossed tissue) divided by (amount initially placed in the upper chamber), multiplied by 100. CV represents the coefficient of variation and is defined as: (standard deviation) divided by (mean), multiplied by 100.

FIG. 2A shows the average fold change of gastrointestinal synthetic epithelial lining (GSEL) permeability, clustered by classes of excipients. Active pharmaceutical ingredient (API) transported is semaglutide. Fold change GSEL (FC GSEL) measures the fold change of permeability of each formulation with excipient, and normalized by the permeability of GSEL only. Each data point represents the average permeability of a separate formulation. The short horizontal bar represents the mean. Data is sorted by the mean permeability of the classes. The value indicates excipient concentration (mg/ml).

FIG. 2B shows the average fold change of gastrointestinal synthetic epithelial lining (GSEL) permeability, clustered by classes of excipients. Active pharmaceutical ingredient (API) transported is tirzepatide. Fold change GSEL (FC GSEL) measures the fold change of permeability of each formulation with excipient, and normalized by the permeability of GSEL only. Each data point represents the average permeability of a separate formulation. The short horizontal bar represents the mean. Data is sorted by the mean permeability of the classes. The value indicates excipient concentration (mg/ml).

FIG. 2C shows the average fold change of gastrointestinal synthetic epithelial lining (GSEL) permeability. Active pharmaceutical ingredient (API) transported is semaglutide. Fold change GSEL (FC GSEL) measures the fold change of permeability of each formulation with excipient, and normalized by the permeability of GSEL only. Each data point represents the average permeability of a separate formulation. The short horizontal bar represents the mean. Data is sorted by the mean permeability of the classes. The numerical value (e.g., 20, 40, 80) on the x-axis indicates excipient concentration (mg/ml).

FIG. 3 shows the percent mean permeability of gastrointestinal synthetic epithelial lining (GSEL) permeability. Active pharmaceutical ingredient (API) transported is tirzepatide. Each data point represents the average permeability of a separate formulation. The short horizontal bar represents the mean. Data is sorted by the mean permeability. The numerical value (e.g., 20, 40, 80) on the x-axis indicates excipient concentration (mg/ml). Table E1 in Example 1 provides a key to the abbreviations used for the excipients, and Table E3 reports the percent permeability (% Pm).

FIG. 4A shows the percent mean permeability of the gastrointestinal synthetic epithelial lining (GSEL) for each combination of excipient. Active pharmaceutical ingredient (API) transported is semaglutide. Fold change GSEL (FC GSEL) measures the fold change of permeability of each formulation with excipient, and normalized by the permeability of GSEL only. Each data point represents the average permeability of a separate formulation. The short horizontal bar represents the mean. Data is sorted by the mean permeability of the classes. The numerical value (e.g., 20, 40, 80) on the x-axis indicates excipient concentration (mg/ml). GSEL_80 represents a doubling of the amount of the ingredients in GSEL_40 except for Tris; GSEL_40 contains 9.8 mg/mL dopamine HCl, 25 mg/ml PDA, 0.67 mg/mL hydrogen peroxide, 2.2 mg/mL Tris HCl, 4.3 mg/mL Tris base, at approximately pH 8.5, and GSEL_80 is 19.6 mg/mL dopamine HCl, 50 mg/ml PDA, 1.34 mg/mL hydrogen peroxide, 2.2 mg/mL Tris HCl, 4.3 mg/mL Tris base (values can vary by plus or minus 5%). The solution was at about pH 8.5.

FIG. 4B shows the percent mean permeability of the gastrointestinal synthetic epithelial lining (GSEL) for each combination of excipient. Active pharmaceutical ingredient (API) transported is semaglutide. Fold change GSEL (FC GSEL) measures the fold change of permeability of each formulation with excipient, and normalized by the permeability of GSEL only. Each data point represents the average permeability of a separate formulation. The short horizontal bar represents the mean. Data is sorted by the mean permeability of the classes. The numerical value (e.g., 20, 40, 80) on the x-axis indicates excipient concentration (mg/ml). GSEL_80 represents a doubling of the amount of the ingredients in GSEL_40 except for Tris; GSEL_40 contains 9.8 mg/mL dopamine HCl, 25 mg/ml PDA, 0.67 mg/mL hydrogen peroxide, 2.2 mg/mL Tris HCl, 4.3 mg/mL Tris base, at approximately pH 8.5, and GSEL_80 is 19.6 mg/mL dopamine HCl, 50 mg/ml PDA, 1.34 mg/mL hydrogen peroxide, 2.2 mg/mL Tris HCl, 4.3 mg/mL Tris base (values can vary by plus or minus 5%). The solution was at about pH 8.5.

FIG. 5A shows the percent mean permeability of the gastrointestinal synthetic epithelial lining (GSEL) permeability for each combination of excipient. Active pharmaceutical ingredient (API) transported is tirzepatide. Percent Pm=(amount that crossed tissue) divided by (amount initially placed in the upper chamber), multiplied by 100. CV represents the coefficient of variation and is defined as: (standard deviation) divided by (mean), multiplied by 100. Each data point represents the average permeability of a separate formulation. The short horizontal bar represents the mean. Data is sorted by the mean permeability of the classes. The numerical value (e.g., 20, 40, 80) on the x-axis indicates excipient concentration (mg/ml).

FIG. 5B shows the percent mean permeability of the gastrointestinal synthetic epithelial lining (GSEL) permeability for each combination of excipient. Active pharmaceutical ingredient (API) transported is tirzepatide. Percent Pm=(amount that crossed tissue) divided by (amount initially placed in the upper chamber), multiplied by 100. CV represents the coefficient of variation and is defined as: (standard deviation) divided by (mean), multiplied by 100. Each data point represents the average permeability of a separate formulation. The short horizontal bar represents the mean. Data is sorted by the mean permeability of the classes. The numerical value (e.g., 20, 40, 80) on the x-axis indicates excipient concentration (mg/ml).

FIG. 5C shows the percent mean permeability of the gastrointestinal synthetic epithelial lining (GSEL) permeability for each excipient. Active pharmaceutical ingredient (API) transported is tirzepatide. Percent Pm=(amount that crossed tissue) divided by (amount initially placed in the upper chamber), multiplied by 100. CV represents the coefficient of variation and is defined as: (standard deviation) divided by (mean), multiplied by 100. Each data point represents the average permeability of a separate formulation. The short horizontal bar represents the mean. Data is sorted by the mean permeability of the classes. The numerical value (e.g., 20, 40, 80) on the x-axis indicates excipient concentration (mg/ml). Table E1 in Example 1 provides a key to the abbreviations used for the excipients, and Table E4 reports the % Pm.

FIG. 5D shows the percent mean permeability of the gastrointestinal synthetic epithelial lining (GSEL) permeability for single excipients and combinations of excipients. Active pharmaceutical ingredient (API) transported is tirzepatide. Percent Pm (% Pm)=(amount that crossed tissue) divided by (amount initially placed in the upper chamber), multiplied by 100. CV represents the coefficient of variation and is defined as: (standard deviation) divided by (mean), multiplied by 100. Each data point represents the average permeability of a separate formulation. The short horizontal bar represents the mean. Data is sorted by the mean permeability of the classes. The numerical value (e.g., 20, 40, 80) on the x-axis indicates excipient concentration (mg/ml). Table E1 in Example 1 provides a key to the abbreviations used for the excipients, and Table E4 reports the % Pm.

FIG. 5E shows the percent mean permeability of the gastrointestinal synthetic epithelial lining (GSEL) for single excipients and combinations of excipients. Active pharmaceutical ingredient (API) transported is tirzepatide. Percent Pm (% Pm)=(amount that crossed tissue) divided by (amount initially placed in the upper chamber), multiplied by 100. CV represents the coefficient of variation and is defined as: (standard deviation) divided by (mean), multiplied by 100. Each data point represents the average permeability of a separate formulation. The short horizontal bar represents the mean. Data is sorted by the mean permeability of the classes. The numerical value (e.g., 20, 40, 80) on the x-axis indicates excipient concentration (mg/ml). Table E1 in Example 1 provides a key to the abbreviations used for the excipients, and Table E5 reports the % Pm.

FIG. 5F shows the percent mean permeability of the gastrointestinal synthetic epithelial lining (GSEL) for single excipients and combinations of excipients. Active pharmaceutical ingredient (API) transported is tirzepatide. Percent Pm (% Pm)=(amount that crossed tissue) divided by (amount initially placed in the upper chamber), multiplied by 100. CV represents the coefficient of variation and is defined as: (standard deviation) divided by (mean), multiplied by 100. Each data point represents the average permeability of a separate formulation. The short horizontal bar represents the mean. Data is sorted by the mean permeability of the classes. The numerical value (e.g., 20, 40, 80) on the x-axis indicates excipient concentration (mg/ml). Table E1 in Example 1 provides a key to the abbreviations used for the excipients, and Table E5 reports the % Pm.

FIG. 6 shows the average fold change of gastrointestinal synthetic epithelial lining (GSEL) permeability for each combination of excipients. The excipient indicated at the top of each graph is kept constant across combinations with the excipients as indicated on the x-axis. The active pharmaceutical ingredient (API) transported is semaglutide. Fold change GSEL (FC GSEL) measures the fold change of permeability of each formulation with excipient, and normalized by the permeability of GSEL only. Each data point represents the average permeability of a separate formulation. The short horizontal bar represents the mean. The long horizontal line across each graph is the mean fold change in permeability of the excipient indicated at the top of the graph. Data is sorted by the mean permeability. The numerical values (e.g., 20, 40, 80) on the x-axis indicate excipient concentrations (mg/ml).

FIG. 7 shows the percent mean permeability of active pharmaceutical ingredient (API) transported (semaglutide) by GSELs with combinations of a bile salt (CHA=Cholic Acid Sodium Salt; GCA=sodium glycocholate) with ammonium carbonate (NHCO) at various concentrations, as well as GSELs with only ammonium carbonate, ammonium carbonate without GSEL, and GSEL without permeation enhancer. Each data point represents the average permeability of a separate formulation. The short horizontal bar represents the mean. Data is sorted by the mean permeability. The numerical values (e.g., 20, 40, 80) on the x-axis indicate excipient concentrations (mg/ml).

FIG. 8 shows the percent mean permeability (% Pm) of active pharmaceutical ingredient (API) (semaglutide). Data is ordered by total mass of CHA (cholic acid sodium salt) and/or NHCO (ammonium carbonate). The numerical values (e.g., 20, 40, 80) on the x-axis indicate excipient concentrations (mg/ml).

FIG. 9 shows the percent mean permeability (Pm) of tirzepatide transported across porcine duodenal tissue 20 hours post delivery as a solid powder. The numerical values (e.g., 40) on the x-axis indicate excipient concentrations (mg/ml).

FIG. 10 shows the percent mean permeability (Pm) of tirzepatide transported across porcine duodenal tissue 20 hours after delivery of GSEL as a liquid suspension. The numerical values (e.g., 40) on the x-axis indicate excipient concentrations (mg/ml).

FIG. 11A shows percent FITC-Dextran-40K (FD40) permeated at 20 hours after GSEL delivery. The numerical values on the x-axis indicate excipient concentrations (mg/ml).

FIG. 11B shows percent FITC-Dextran-40K (FD40) permeated at 6 hours and 20 hours after GSEL delivery.

FIG. 12A shows amount FITC-Dextran-4K (FD4) permeated (μg) over time after GSEL delivery with or without various ratios of permeation enhancers (PE, where the permeation enhancer is a 1:1 combination of sodium glycocholate (GCA) and ammonium carbonate (NHCO), at 40 mg/ml each, i.e. GCA-NHCO_40-40).

FIG. 12B shows amount FITC-Dextran-4K (FD4) permeated (μg) over time after delivery of various GSEL formulations. The values indicate ratios.

FIG. 12C shows a table summarizing the amount FITC-Dextran-4K (FD4) (μg) permeated over time after delivery of various GSEL formulations. The values indicate ratios.

FIG. 12D shows Franz cell FITC-Dextran-4K (FD4) permeation experiments with or without permeation enhancer. **P=0.029, Confidence level=95%, n=7.

FIG. 13 shows the percent mean permeability (Pm) of active pharmaceutical ingredient (API) (semaglutide). Porcine colon tissue is used as the barrier.

FIG. 14 shows the percent mean permeability of semaglutide in a Franz cell experiment comparing bile salts alone and in combination with other excipients as permeation enhancers.

FIG. 15A shows the percent mean permeability of semaglutide over twenty hours in a Franz cell test comparing a combination of sodium glycocholate (GCA) and ammonium carbonate (NHCO) versus a combination of sodium glycocholate (GCA) and sodium carbonate (NaCO) as a permeation enhancer.

FIG. 15B shows the amount (μg) of semaglutide over seven hours in a Franz cell test comparing a combination of sodium glycocholate (GCA) and ammonium carbonate (NHCO) versus a combination of sodium glycocholate (GCA) and sodium carbonate (NaCO) as a permeation enhancer. FIG. 15B is an expanded view of the initial hours of the graph shown in FIG. 15C.

FIG. 15C shows the amount (μg) of semaglutide permeated over twenty hours in a Franz cell test comparing a combination of sodium glycocholate (GCA) and ammonium carbonate (NHCO) versus a combination of sodium glycocholate (GCA) and sodium carbonate (NaCO) as a permeation enhancer. FIG. 15B is an expanded view of the initial hours of the graph shown in FIG. 15C.

FIG. 15D shows the percent mean permeability calculated for semaglutide permeated over twenty hours in a Franz cell test comparing a combination of sodium glycocholate (GCA) and ammonium carbonate (NHCO) versus a combination of sodium glycocholate (GCA) and sodium carbonate (NaCO) as permeation enhancers.

FIG. 16A shows the percent mean permeability of semaglutide in a comparison of various ammonium salts, such as ammonium carbonate (NHCO), ammonium sulfate (NHSO), and ammonium bicarbonate (NHHCO) salts in combination with Cholic Acid Sodium Salt (CHA) as a permeation enhancer.

FIG. 16B shows the values of determined percent mean permeability of semaglutide in a comparison of various ammonium carbonate (NHCO) salts in combination with Cholic Acid Sodium Salt (CHA) as a permeation enhancer after 20 hours.

FIG. 17A shows the percent mean permeability of semaglutide in a comparison of bile salts taurocholic acid (TCA) and taurochenodeoxycholic acid (TCDCA) as a permeation enhancer.

FIG. 17B shows the values of determined percent mean permeability of semaglutide in a comparison of bile salts as a permeation enhancer after 20 hours.

FIG. 18A shows the percent mean permeability of semaglutide in a comparison of cholic acid (CHA) with NHCO or SNAC and GCA with SNAC as a permeation enhancer after 20 hours.

FIG. 18B shows the values of determined percent mean permeability of semaglutide in a comparison of cholic acid (CHA) with NHCO or SNAC and GCA with SNAC as a permeation enhancer after 20 hours.

FIG. 19A shows the percent mean permeability of semaglutide across duodenal tissue in a comparison of GCA and NHCO, or GCA, NHCO and GSEL as a permeation enhancer after 20 hours.

FIG. 19B shows the values of determined percent mean permeability of semaglutide across duodenal tissue in a comparison of GCA and NHCO, or GCA, NHCO and GSEL as permeation enhancers after 20 hours.

FIG. 20A shows that percent coverage of surface by GSEL does not increase significantly after 30 minutes of incubation.

FIG. 20B shows a schematic for Franz cell experiments that test co-administration versus co-formulation.

FIG. 20C shows the percent dye (FITC-Dextran-4K) entrapped FITC-Dextran-4K on duodenum tissue with and without GSEL and/or CHA (as permeation enhancer), where the formulation is administered as a liquid (e.g., suspension) compared to solid formulation. ****P<0.0001 using one-way ANOVA. Confidence interval: 95%, n=4-8.

FIG. 20D shows the percent dye (FITC-Dextran-4K) permeated on duodenum tissue when administered as a liquid (e.g., suspension) compared to a solid formulation. * indicates p<0.05.

FIG. 20E shows the percent dye (FITC-Dextran 4K, FITC-Dextran 10K, or FITC-Dextran 40K) entrapped on porcine duodenal tissue in the presence of GSEL, or GSEL and permeation enhancer (CHA). Values were compared using 2-way ANOVA followed by Dunnett's multiple comparisons test. **** indicates p<0.0001. The table summarizes the percent of FITC-Dextran 4K, FITC-Dextran 10K, or FITC-Dextran 40K entrapped on the tissue in the presence of GSEL, or GSEL and permeation enhancer (CHA). Formulation was delivered as a liquid (e.g., suspension).

FIG. 20F shows the percent dye (FITC-Dextran 4K, FITC-Dextran 10K, or FITC-Dextran 40K) permeated through porcine duodenal in the presence of GSEL, or GSEL and permeation enhancer (CHA). Values were compared using 2-way ANOVA followed by Sidak's multiple comparisons test. **** indicates p<0.0001; *** indicates p<0.0002. Formulation was delivered as a liquid (e.g., suspension). The table summarizes the percent of FITC-Dextran 4K, FITC-Dextran 10K, or FITC-Dextran 40K permeated through the tissue in the presence of GSEL, or GSEL and permeation enhancer (CHA). Formulation was delivered as a liquid (e.g., suspension).

FIG. 20G shows the results of a screen with bile salts with GSEL platform on Franz cells, where GSEL is composed of 6.25 mg PDA, 2.45 mg DA-HCl, with a 14.6:1 DA-HCl:H₂O₂ ratio and a 4 μL/mm² dispensing volume and CHA was tested at 40 mg/mL (mass 10 mg), n=4-8, Semaglutide was quantified using ELISA. Formulation was delivered as a liquid (e.g., suspension).

FIG. 20H shows the results of a permeability test where GSEL was combined with the bile salt CHA at increasing concentrations, and GSEL was composed of 6.25 mg PDA, 2.45 mg DA-HCl, with a 14.6:1 DA-HCl:H₂O₂ ratio and a 4 μL/mm² dispensing volume and CHA was tested in the range 40-160 mg/mL (mass 10-40 mg), n=7-24, Semaglutide was quantified using ELISA. Formulation was delivered as a liquid (e.g., suspension).

FIG. 21 shows the set up of a dynamic colocalization test conducted on porcine jejunal tissue (6 inches in length) at an angle of 17.92° for FD4 or semaglutide with and without GSEL and at different GSEL concentrations. The suspensions are dispensed at 3 mL/min and the flow-through is collected to analyze how much FD4 or semaglutide does not colocalize.

FIG. 22A shows the results of a Franz cell permeability test where GSEL is combined with bile salt CHA and different ammonium salts on Franz cells, where GSEL is composed of 6.25 mg PDA, 2.45 mg DA-HCl, with a 14.6:1 DA-HCl:H₂O₂ ratio and a 4 μL/mm² dispensing volume and the ammonium salts were tested at 40 mg/mL (mass 10 mg), GSEL concentration, n=7-24, Semaglutide was quantified using ELISA.

FIG. 22B shows the results of a Franz cell permeability test where GSEL is combined with bile salt CHA and SNAC or other benzene ring-based excipients, where GSEL is composed of 6.25 mg PDA, 2.45 mg DA-HCl, with a 14.6:1 DA-HCl:H₂O₂ ratio and a 4 μL/mm² dispensing volume and the benzene ring-based excipients were tested at 40 mg/mL (mass 10 mg), n=7-24, Semaglutide was quantified using ELISA.

FIG. 22C shows the results of permeability tests on Franz cells where GSEL:permeation enhancer (PE):semaglutide (SEMA) (PE=GCA−NHCO) is compared against controls; semaglutide only, semaglutide with SNAC at 14:300, SEMA with GCA-NHCO, SEMA with GSEL, The GSEL composition is composed of 6.25 mg PDA, 2.45 mg DA-HCl, with 14.6:1 DA-HCl:H₂O₂ ratio and 4 L/mm² dispensing volume, GCA and NHCO are tested at 40 mg/mL (mass 10 mg). ****P<0.0001, using one-way ANOVA, Confidence interval: 95%, n=8.

FIG. 22D shows the results of permeability tests on Franz cells where 1×GSEL includes 6.25 mg PDA and 2.45 mg DA-HCl, with 14.6:1 DA-HCl:H₂O₂ ratio, 2 L/mm² dispensing volume and PE mass (GCA+NHCO) is 2, 10, 20, or 40 mg, **P=0.0016, ****P<0.0001, using one-way ANOVA, Confidence interval: 95%, n=22.

FIG. 22E shows the results of permeability tests on Franz cells where PE mass is 20 mg (10 mg GCA+10 mg NHCO) and PDA and DA-HCl masses change (1×=PDA 6.25 mg and DA-HCl=2.45 mg, 2×=PDA 12.5 mg, DA-HCl=4.9 mg, 4×=PDA 25 mg, DA-HCl=9.8 mg, 14.6:1 DA-HCl:H₂O₂ ratio, 2 μL/mm² dispensing volume), P=0.6906, *P=0.0498, using one-way ANOVA, Confidence interval: 95%, n=8-12.

FIG. 22F shows dynamic colocalization tests conducted on porcine jejunal tissue for FD4 with and without GSEL and at different GSEL concentrations where permeation enhancer (PE): 40 mg/mL GCA+40 mg/mL NHCO; and the permeation enhancer:semaglutide ratio=20:0.5; PDA 25 mg/mL and DA-HCl-9.8 mg/mL for 1×GSEL; PDA 50 mg/mL and DA-HCl=19.6 mg/mL for 2×GSEL, 14.6:1 DA-HCl:H₂O₂ ratio, 3 mL through 6 inches tissue over 1 minute at 17.92°, ****P<0.0001 using one-way ANOVA, Confidence interval: 95%, n=3.

FIG. 22G shows static colocalization tests on porcine duodenum tissue for FD4, PE 20 mg (10 mg GCA+10 mg NHCO), PDA 6.25 mg and DA-HCl=2.45 mg for 1×GSEL, PDA 12.5 mg and DA-HCl=4.9 mg for 2×GSEL, 14.6:1 DA-HCl:H₂O₂ ratio, 2 L/mm² dispensing volume, ***P=0.0004, ****P<0.0001, using one-way ANOVA, Confidence interval: 95%, n=4.

FIG. 22H shows the results of a dynamic colocalization test on porcine jejunal tissue, for semaglutide with and without GSEL and at different permeation enhancer (PE) concentrations where 1×PE (40 mg/mL GCA+40 mg/mL NHCO), or 0.1×PE (4 mg/mL GCA+4 mg/mL NHCO), PDA 50 mg/mL and DA-HCl=19.6 mg/mL, 14.6:1 DA-HCl:H₂O₂ ratio, 3 mL through 6 inches of tissue over 1 minute at 17.92°, n=2.

FIG. 22I shows the porcine jejunal tissue after the dynamic colocalization test using FD4.

FIG. 22J shows the permeability of semaglutide (mass, ug) on Franz cells for different permeation enhancer:semaglutide ratios (20:0.5, 20:1, 20:2, 20:4, 20:8) for 2×GSEL (PDA 12.5 mg, DA-HCl=4.9 mg, 14.6:1 DA-HCl:H₂O₂ ratio, 2 μL/mm² dispensing volume), and 1× permeation enhancer (PE) (GCA 10 mg, NHCO 10 mg (total PE 20 mg)), and semaglutide is increased **P=0.0044, ****P<0.0001 using one-way ANOVA, confidence interval: 95%, n=8-9. The permeation enhancer:semaglutide ratio was changed by modulating the amount of semaglutide inside.

FIG. 22K shows the results of a static colocalization test on porcine duodenum tissue for semaglutide, where the total mass of permeation enhancer used is 20 mg (10 mg GCA+10 mg NHCO), permeation enhancer:semaglutide ratio is 20:2, PDA 6.25 mg and DA-HCl=2.45 mg for 1×GSEL, PDA 12.5 mg and DA-HCl=4.9 mg for 2×GSEL, 14.6:1 DA-HCl:H₂O₂ ratio, 2 L/mm² dispensing volume, *P=0.0155, using one-way ANOVA, Confidence interval: 95%, n=4-10.

FIG. 22L shows the results of the dynamic colocalization test on porcine jejunal tissue for semaglutide, PE: 40 mg/mL GCA+40 mg/mL NHCO), PE:SEMA=20:0.5, PDA 25 mg/mL and DA-HCl=9.8 mg/mL for 1×GSEL, PDA 50 mg/mL and DA=19.6 mg/mL for 2×GSEL, 14.6:1 DA-HCl:H₂O₂ ratio, 4×GSEL=PDA 25 mg, DA-HCl=9.8 mg, 14.6:1 DA-HCl:H₂O₂ ratio; 3 mL through 6 inches of tissue over 1 minute at 17.92°, **P=0.0286, using unpaired two-tailed t-test, no significant difference using one-way ANOVA, Confidence interval: 95%, n=4.

FIG. 22M shows the results of a Franz cell permeability of semaglutide (%) on Franz cells for different permeation enhancer:semaglutide ratios for 2×GSEL, for different permeation enhancer:semaglutide ratios (20:0.5, 20:1, 20:2, 20:4, 20:8) where 2×GSEL is composed of PDA 12.5 mg, DA-HCl=4.9 mg, 14.6:1 DA-HCl:H₂O₂ ratio, and 1×PE (GCA 10 mg, NHCO 10 mg (total PE 20 mg)), 2 L/mm² dispensing volume, no significant change for any of the groups, using one-way ANOVA, Confidence interval: 95%, n=8.

FIG. 23A shows the pharmacokinetic curve for intravenous administration of 1 mg of semaglutide per pig. N=2

FIG. 23B shows the pharmacokinetic curves for the control groups (e.g., 1× permeation enhancer alone) and experimental groups that received formulation by endoscopic placement to the duodenum (2×GSEL combined with 1× permeation enhancer).

FIG. 23C shows the pharmacokinetic curves for the control groups (e.g., 1× permeation enhancer alone) and experimental groups that received formulation by endoscopic administration (2×GSEL combined with 1× permeation enhancer). FIG. 23C is a zoomed in version of FIG. 23B.

FIG. 23D shows the AUC values for up to 24 hours for control, (semaglutide alone), Control (1× permeation enhancer alone), 1×GSEL combined with 1× permeation enhancer (1×GSEL_1×PE) and 2×GSEL combined with 1× permeation enhancer (2×GSEL_1×PE). 1×PE corresponds to 6.4 mg/kg GCA and 6.4 mg/kg NHCO, 1×GSEL includes 4.55 mg/kg PDA and 1.8 mg/kg DA-HCl, 2×GSEL includes 9.1 mg/kg PDA and 3.6 mg/kg DA-HCl.

FIG. 23E shows the AUC values for up to 48 hours for control, (semaglutide alone), Control (1× permeation enhancer alone), 1×GSEL combined with 1× permeation enhancer (1×GSEL_1×PE) and 2×GSEL combined with 1× permeation enhancer (2×GSEL_1×PE). 1×PE corresponds to 6.4 mg/kg GCA and 6.4 mg/kg NHCO, 1×GSEL includes 4.55 mg/kg PDA and 1.8 mg/kg DA-HCl, 2×GSEL includes 9.1 mg/kg PDA and 3.6 mg/kg DA-HCl.

FIG. 23F shows the AUC values for up to 72 hours for control, (semaglutide alone), Control (1× permeation enhancer alone), 1×GSEL combined with 1× permeation enhancer (1×GSEL_1×PE) and 2×GSEL combined with 1× permeation enhancer (2×GSEL_1×PE). 1×PE corresponds to 6.4 mg/kg GCA and 6.4 mg/kg NHCO, 1×GSEL includes 4.55 mg/kg PDA and 1.8 mg/kg DA-HCl, 2×GSEL includes 9.1 mg/kg PDA and 3.6 mg/kg DA-HCl.

FIG. 23G shows the AUC values for up to 168 hours for control, (semaglutide alone), Control (1× permeation enhancer alone), 1×GSEL combined with 1× permeation enhancer (1×GSEL_1×PE) and 2×GSEL combined with 1× permeation enhancer (2×GSEL_1×PE). 1×PE corresponds to 6.4 mg/kg GCA and 6.4 mg/kg NHCO, 1×GSEL includes 4.55 mg/kg PDA and 1.8 mg/kg DA-HCl, 2×GSEL includes 9.1 mg/kg PDA and 3.6 mg/kg DA-HCl.

### DETAILED DESCRIPTION OF THE INVENTION

The present disclosure relates to synthetic linings formed in situ in the gastrointestinal tract, such as in the small intestine. Such a lining is called a Gastrointestinal Synthetic Epithelial Lining (GSEL). A GSEL is a temporary polymeric lining that rests against (i.e., contacts) the internal surface of the gastrointestinal tract. For example, dopamine can be polymerized in the small intestine to form a GSEL coating in the interior of the duodenum, jejunum, or ileum. The present disclosure also describes the use of GSELs in combination with an agent and one or more permeation enhancers. The one or more permeation enhancers increase the absorption of the agent across the intestinal tissue, as compared to the GSEL without permeation enhancer.

GSELs can be used to localize an active pharmaceutical ingredient (API) in a specific location, resulting in prolonged contact with the gastrointestinal mucosa. Due to the prolonged contact with the mucosa, the API can be delivered over an extended period, which can be advantageous for APIs that are absorbed slowly, or which are best administered over a lengthy time period. A GSEL can permit a higher percentage of an API to be absorbed as compared to a standard dosage form, and this higher percentage of absorption with a GSEL can allow attainment of similar total API absorption with administration of a lower dosage of API. A GSEL can also reduce the frequency of administration of an API by provided for extended release of API, as compared to multiple administrations of a conventional dose formulation over a period of time.

Despite the prolonged contact afforded by a GSEL, some APIs may not permeate through the gastrointestinal barrier in sufficient amounts to be therapeutically effective, or may not permeate at all. The present disclosure describes permeation enhancers for use in GSELs which enhance the permeation of an active pharmaceutical ingredient (API) across the gastrointestinal mucosa, as compared to GSELs lacking a permeation enhancer. The present disclosure also describes permeation enhancers for use in GSELs which enhance the permeation of an active pharmaceutical ingredient (API) across the gastrointestinal mucosa, as compared to GSELs lacking a permeation enhancer, and which can be used to increase permeation of APIs that may otherwise not penetrate in sufficient amounts GSELs with one or more permeation enhancers can provide for efficient and effective administration of APIs via enteral administration.

In order to form a GSEL, a GSEL composition is administered to a subject orally. The composition can alternatively be administered by feeding tube, for example, in subjects with impaired swallowing ability; or, if necessary, the composition can be administered using an endoscope to deliver the composition to the small intestine, stomach, or esophagus. The GSEL composition for administration comprises a polymer precursor (e.g., a monomer, oligomer, or other prepolymer, such as dopamine, dopamine oligomers, or dopamine prepolymers). This polymer precursor is polymerized to form the GSEL. The GSEL composition for administration also comprises an oxygen source (such as hydrogen peroxide or urea hydrogen peroxide), which will provide oxygen at the appropriate location in the gastrointestinal tract to cause polymerization of the polymer precursor. The GSEL composition for administration also comprises one or more permeation enhancers as described herein (such as sodium caprate, sodium caprylate, sodium glycocholate and/or ammonium carbonate), to increase the permeation of the active pharmaceutical ingredient across the gastrointestinal barrier. The composition relies on one or more catalysts endogenous to the gastrointestinal tract to convert the oxygen precursor to oxygen. Liberation of oxygen in turn leads to polymerization of the polymer precursor. The small intestine contains endogenous catalysts with peroxidase activity such as catalase, which enables liberation of oxygen, resulting in formation and deposition of the GSEL in the specific anatomical location where the endogenous catalyst is located. The GSEL composition for administration can also include an active pharmaceutical ingredient (API). Formation of the GSEL with its associated API thus localizes the API and the one or more permeation enhancers to the vicinity of the polymer. In this manner, a depot of the API and the one or more permeation enhancers can be retained at a specific anatomical location over an extended period of time for absorption at that location. For example, when the GSEL containing API and the one or more permeation enhancers forms in the small intestine, the GSEL keeps the API in proximity to the intestinal epithelium for an extended period. In contrast, when an API is administered as a tablet which dissolves in the intestinal tract, without a GSEL, the API may transit the small intestine much more rapidly, leading to lower absorption of API, or requiring much more API to be administered to obtain equivalent absorption.

Alternatively, the API can be administered in a separate composition from the GSEL precursor composition. If the API is administered in a separate composition, it can be administered before administration of the GSEL precursor composition, concurrently with administration of the GSEL precursor composition, or subsequent to administration of the GSEL precursor composition.

The GSEL with the one or more permeation enhancers aids the API in crossing the gastrointestinal barrier, leading to increased permeation of the API across the gastrointestinal barrier in the presence of the one or more permeation enhancers compared to permeation of the API using a GSEL without permeation enhancer. Even if the API is administered separately from the GSEL and the one or more permeation enhancers, enhanced permeation of the API can occur when the API traverses the portion of the small intestine coated by the GSEL with the one or more permeation enhancers.

The components of the GSEL composition for administration are discussed in more detail herein, as follows: 1) polymer precursors, 2) oxygen sources, and 3) permeation enhancers. GSELs and GSEL compositions for administration may also contain 4) one or more active pharmaceutical ingredients (APIs). A GSEL or GSEL composition for administration may not contain any API, in which case the one or more APIs can be administered separately. Alternatively, a GSEL or GSEL composition for administration may contain one or more APIs, and one or more APIs can optionally also be administered separately from the GSEL. Also discussed are endogenous catalysts which allow for formation of the GSEL in situ.

### Definitions

An “agent” is any substance intended for therapeutic, diagnostic, or nutritional use in a patient, individual, or subject. Agents include, but are not limited to, drugs, nutrients, vitamins, and minerals. An “active pharmaceutical ingredient” (API) is an agent which is the component in a pharmaceutical dosage that produces the desired biological effect on a patient, individual, or subject to whom the dosage is administered.

An “excipient” is any substance added to a formulation of an agent that is not the agent itself. Excipients include, but are not limited to, binders, coatings, diluents, disintegrants, emulsifiers, flavorings, glidants, lubricants, and preservatives.

A “patient,” “individual,” or “subject” refers to a human, or a non-human animal. Non-human animals include mammals, including domestic animals such as a dog or cat; or an animal used in commerce, such as a cow, pig, horse, sheep, or goat. In a preferred embodiment, a patient, individual, or subject is a human.

“Treating” a disease or disorder with the compositions and methods disclosed herein is defined as administering one or more of the compositions disclosed herein to a patient in need thereof, with or without additional agents, in order to reduce or eliminate either the disease or disorder, or one or more symptoms of the disease or disorder, or to suppress symptoms of the disease or disorder, or to retard the progression of the disease or disorder or of one or more symptoms of the disease or disorder, or to reduce the severity of the disease or disorder or of one or more symptoms of the disease or disorder. Treatment can commence after emergence of symptoms of a disease or disorder, or can commence before emergence of symptoms of a disease or disorder. Treatment can also be continued after symptoms have been resolved.

“Therapeutic use” of the compositions disclosed herein is defined as using one or more of the compositions disclosed herein to treat a disease or disorder, as defined above. A “therapeutically effective amount” of a therapeutic agent, such as a drug, is an amount of the agent, which, when administered to a patient, individual, or subject, is sufficient to achieve the desired biological result, such as an amount sufficient to reduce or eliminate either a disease or disorder or one or more symptoms of a disease or disorder, or to retard the progression of a disease or disorder or of one or more symptoms of a disease or disorder, or to reduce the severity of a disease or disorder or of one or more symptoms of a disease or disorder. A therapeutically effective amount can be administered to a patient, individual, or subject as a single dose, or can be divided and administered as multiple doses.

“Permeation” of an agent or of an active pharmaceutical ingredient (API) across a barrier refers to the amount of the agent or API able to cross the barrier.

A “permeation enhancer” refers to a substance which is able to increase the permeation of an agent or an active pharmaceutical ingredient across a barrier.

The term “polymer” refers to a compound comprising eleven or more covalently connected repeating units.

The term “oligomer” refers to a compound comprising between two to ten covalently connected repeating units.

The unit “kD” refers to kilodaltons.

As used herein, the singular forms “a”, “an”, and “the” include plural references unless indicated otherwise or the context clearly dictates otherwise.

When numerical values are expressed herein using the term “about” or the term “approximately,” it is understood that both the value specified, as well as values reasonably close to the value specified, are included. For example, the description “about 50° C.” or “approximately 50° C.” includes both the disclosure of 50° C. itself, as well as values close to 50° C. Thus, the phrases “about X” or “approximately X” include a description of the value X itself. If a range is indicated, such as “approximately 50° C. to 60º C” or “about 50° C. to 60° C.,” it is understood that both the values specified by the endpoints are included, and that values close to each endpoint or both endpoints are included for each endpoint or both endpoints; that is, “approximately 50° C. to 60° C.” (or “about 50° C. to 60° C.”) is equivalent to reciting both “50° C. to 60° C.” and “approximately 50° C. to approximately 60º C” (or “about 50° C. to 60° C.”).

With respect to numerical ranges disclosed in the present description, any disclosed upper limit for a component may be combined with any disclosed lower limit for that component to provide a range (provided that the upper limit is greater than the lower limit with which it is to be combined). Each of these combinations of disclosed upper and lower limits are explicitly envisaged herein. For example, if ranges for the amount of a particular component are given as 10% to 30%, 10% to 12%, and 15% to 20%, the ranges 10% to 20% and 15% to 30% are also envisaged, whereas the combination of a 15% lower limit and a 12% upper limit is not possible and hence is not envisaged.

Unless otherwise specified, percentages of ingredients in compositions are expressed as weight percent, or weight/weight percent. It is understood that reference to relative weight percentages in a composition assumes that the combined total weight percentages of all components in the composition add up to 100. It is further understood that relative weight percentages of one or more components may be adjusted upwards or downwards such that the weight percent of the components in the composition combine to a total of 100, provided that the weight percent of any particular component does not fall outside the limits of the range specified for that component.

Some embodiments described herein are recited as “comprising” or “comprises” with respect to their various elements. In alternative embodiments, those elements can be recited with the transitional phrase “consisting essentially of” or “consists essentially of” as applied to those elements. In further alternative embodiments, those elements can be recited with the transitional phrase “consisting of” or “consists of” as applied to those elements. Thus, for example, if a composition or method is disclosed herein as comprising A and B, the alternative embodiment for that composition or method of “consisting essentially of A and B” and the alternative embodiment for that composition or method of “consisting of A and B” are also considered to have been disclosed herein. Likewise, embodiments recited as “consisting essentially of” or “consisting of” with respect to their various elements can also be recited as “comprising” as applied to those elements. Finally, embodiments recited as “consisting essentially of” with respect to their various elements can also be recited as “consisting of” as applied to those elements, and embodiments recited as “consisting of” with respect to their various elements can also be recited as “consisting essentially of” as applied to those elements.

When a composition or system is described as “consisting essentially of” the listed elements, the composition or system contains the elements expressly listed, and may contain other elements which do not materially affect the condition being treated (for compositions for treating conditions), or the properties of the described system (for compositions comprising a system). However, the composition or system either does not contain any other elements which do materially affect the condition being treated other than those elements expressly listed (for compositions for treating systems) or does not contain any other elements which do materially affect the properties of the system (for compositions comprising a system); or, if the composition or system does contain extra elements other than those listed which may materially affect the condition being treated or the properties of the system, the composition or system does not contain a sufficient concentration or amount of those extra elements to materially affect the condition being treated or the properties of the system. When a method is described as “consisting essentially of” the listed steps, the method contains the steps listed, and may contain other steps that do not materially affect the condition being treated by the method or the properties of the system produced by the method, but the method does not contain any other steps which materially affect the condition being treated or the system produced other than those steps expressly listed.

This disclosure provides several embodiments. It is contemplated that any features from any embodiment can be combined with any features from any other embodiment where possible. In this fashion, hybrid configurations of the disclosed features are within the scope of the present disclosure.

### Gastrointestinal Synthetic Epithelial Linings (GSELs) for Administration of Agents

The gastrointestinal synthetic epithelial lining (GSEL) precursor compositions for formation of the GSEL comprise 1) a polymer precursor; 2) an oxygen source; and 3) one or more permeation enhancers. In one embodiment, each component of the composition can be administered as a separate dosage form, to combine in the gastrointestinal tract of a subject. In one embodiment, the polymer precursor and oxygen source can be administered together in one dosage form, and the one or more permeation enhancers can be administered in a separate dosage form. In one embodiment, the polymer precursor, oxygen source, and one or more permeation enhancers can be administered together in one dosage form.

The GSEL precursor composition can also comprise 4) an agent, such as an active pharmaceutical ingredient (API); or the GSEL precursor composition can comprise multiple agents such as multiple APIs. As with the polymer precursor, oxygen source, and one or more permeation enhancers, the agent can be administered together with the other components of the GSEL precursor composition. Alternatively, an API (or multiple APIs) can be administered in a separate composition, either before administration of the other components of the GSEL precursor composition, concurrently with administration of the other components of the GSEL precursor composition, or subsequent to administration of the other components of the GSEL precursor composition.

### Polymer Precursors for Gastrointestinal Synthetic Epithelial Lining Precursor Compositions

In one aspect, the disclosure provides a method of forming a polymer in situ in a subject, the method comprising administering to a subject a composition comprising a polymer precursor, an oxygen source, and one or more permeation enhancers, wherein the polymer precursor and the oxygen source contact a catalyst endogenous to the subject and the catalyst polymerizes the polymer precursor.

In some embodiments, polymerization of the polymer precursor is catalyzed when the oxygen source and polymer precursor contact the endogenous catalyst. In some embodiments, polymerization of the polymer precursor is catalyzed when the oxygen source and polymer precursor contact a peroxidase in the small intestine. In some embodiments, polymerization of the polymer precursor is catalyzed when the oxygen source and polymer precursor contact a peroxidase in the duodenum.

In some embodiments, the polymer precursor is a catechol-based polymer precursor. In some embodiments, the polymer precursor comprises a 1,2-dihydroxybenzene moiety. In some embodiments, the polymer precursor comprises an optionally substituted 1,2-dihydroxybenzene moiety. In some embodiments, the polymer precursor comprises a 1,2-dihydroxyphenyl moiety. In some embodiments, the polymer precursor comprises an optionally substituted 1,2-dihydroxyphenyl moiety.

In some embodiments, the polymer precursor is a 2-(3,4-dihydroxyphenyl)ethylamine-based polymer precursor. (2-(3,4-dihydroxyphenyl)ethylamine is commonly known as dopamine.) In certain embodiments, the polymer precursor comprises a 3,4-dihydroxyphenethylamine moiety. In certain embodiments, the polymer precursor comprises an optionally substituted 3,4-dihydroxyphenethylamine moiety. In certain embodiments, the polymer precursor comprises 2-(3,4-dihydroxyphenyl)ethylamine. In certain embodiments, the polymer precursor comprises an optionally substituted 2-(3,4-dihydroxyphenyl)ethylamine moiety.

In some embodiments, the polymer precursor is selected from the group consisting of dopamine, levodopa, norepinephrine, methyldopa, levodopa methyl ester, levodopa cthyl ester, derivatives thereof, and combinations thereof. In certain embodiments, the polymer precursor is selected from the group consisting of dopamine, levodopa, norepinephrine, methyldopa, levodopa methyl ester, and levodopa ethyl ester.

In certain embodiments, the polymer precursor is a derivative of dopamine. In some embodiments, the polymer precursor comprises dopamine, levodopa, norepinephrine, methyldopa, levodopa methyl ester, or levodopa ethyl ester.

In some embodiments, the derivative of the polymer precursor comprises a macromolecule. In some embodiments, the polymer precursor comprises dopamine, levodopa, norepinephrine, methyldopa, levodopa methyl ester, or levodopa ethyl ester and at least one macromolecule. In certain embodiments the macromolecule is alginate, hyaluronic acid, polyacrylic acid, polyethylene glycol, chondroitin sulfate, chitosan, or a combination thereof. In some embodiments the macromolecule is alginate, hyaluronic acid, polyacrylic acid, polyethylene glycol, chondroitin sulfate, or chitosan. In certain embodiments the polymer precursor comprises (A) dopamine, levodopa, norepinephrine, methyldopa, levodopa methyl ester, or levodopa ethyl ester moieties bound to (B) alginate, hyaluronic acid, polyacrylic acid, polyethylene glycol, chondroitin sulfate, or chitosan. In certain embodiments the polymer precursor comprises (A) more than one of dopamine, levodopa, norepinephrine, methyldopa, levodopa methyl ester, and levodopa ethyl ester moieties bound to (B) alginate, hyaluronic acid, polyacrylic acid, polyethylene glycol, chondroitin sulfate, or chitosan. In certain embodiments the polymer precursor comprises (A) more than one of dopamine, levodopa, norepinephrine, methyldopa, levodopa methyl ester, and levodopa ethyl ester moieties bound to (B) more than one of alginate, hyaluronic acid, polyacrylic acid, polyethylene glycol, chondroitin sulfate, and chitosan.

In certain embodiments, the polymer precursor is selected from the structures listed in Table 1 or 2.

TABLE 1
Polymer precursors

| Polymer | |
|---|---|
| precursor | Structure |
| M1 | 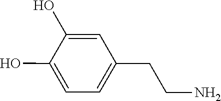 [SMILES: NCCc1ccc(O)c(O)c1] |
| M2 | 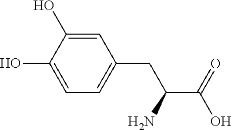 [SMILES: N[C@@H](Cc1ccc(O)c(O)c1)C(=O)O] |
| M3 | 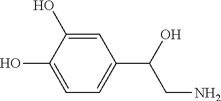 [SMILES: NCC(O)c1ccc(O)c(O)c1] |
| M4 | 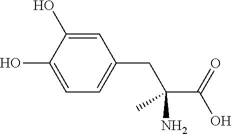 |
| M5 | 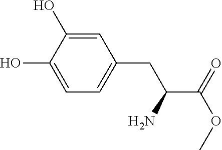 |
| M6 | 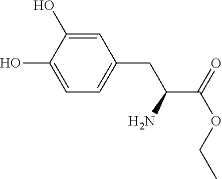 [SMILES: CCOC(=O)[C@@H](N)Cc1ccc(O)c(O)c1] |

TABLE 2
Macromolecules for Use as Polymer Precursors

| Macromolecule | Components | Structure |
|---|---|---|
| C1 | M1 and alginate | 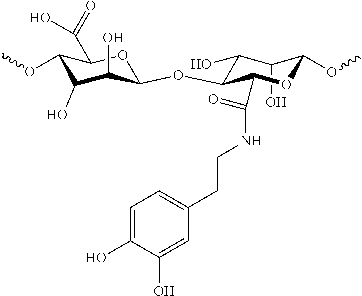 |
| C2 | M2 and alginate | 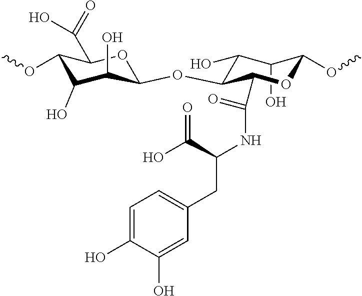 |
| C3 | M3 and alginate | 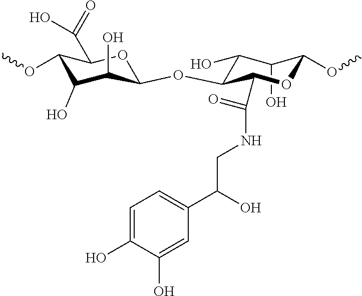 |
| C4 | M4 and alginate | 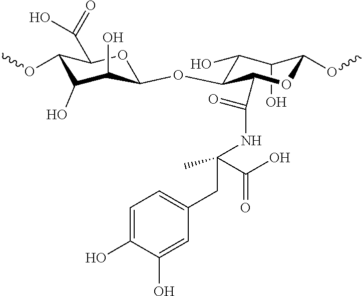 |
| C5 | M5 and alginate | 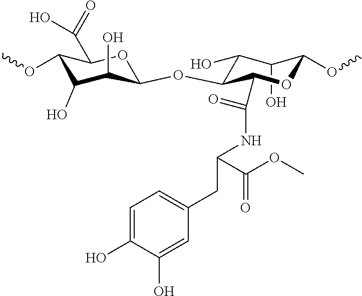 |
| C6 | M6 and alginate | 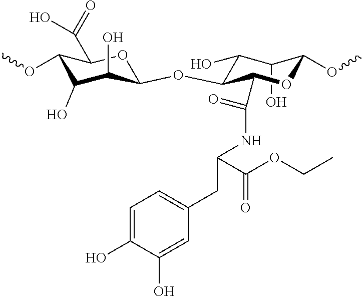 |
| C7 | M1 and hyaluronic acid | 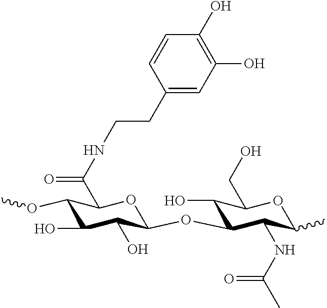 |
| C8 | M2 and hyaluronic acid | 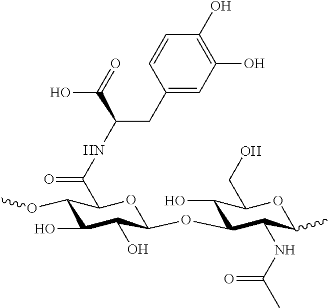 |
| C9 | M3 and hyaluronic acid | 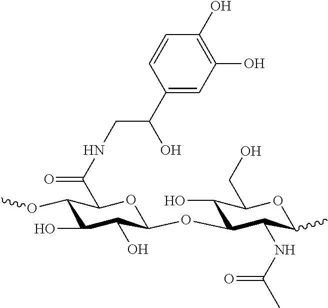 |
| C10 | M4 and hyaluronic acid | 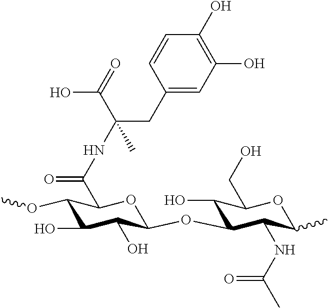 |
| C11 | M5 and hyaluronic acid | 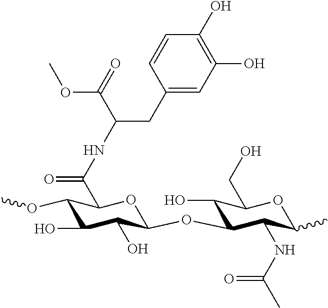 |
| C12 | M6 and hyaluronic acid | 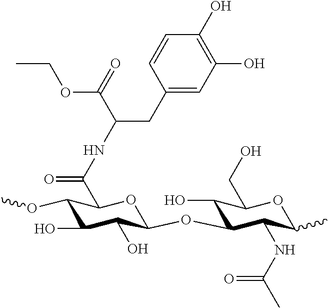 |
| C13 | M1 and polyacrylic acid | 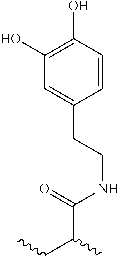 |
| C14 | M2 and polyacrylic acid | 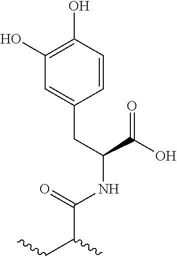 |
| C15 | M3 and polyacrylic acid | 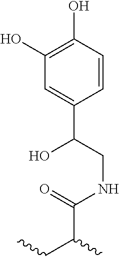 |
| C16 | M4 and polyacrylic acid | 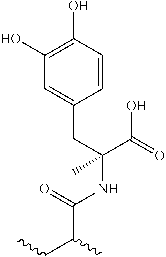 |
| C17 | M5 and polyacrylic acid | 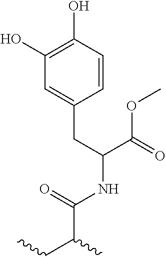 |
| C18 | M6 and polyacrylic acid | 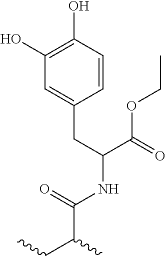 |
| C19 | M1 and polyethylene with tripentaerythritol core | 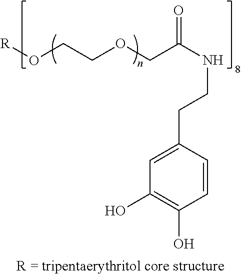 |
| C20 | M1 and chondroitin sulfate | 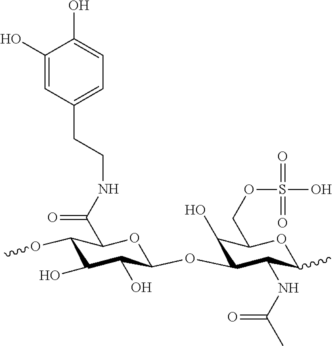 |
| C21 | M1 and chitosan | 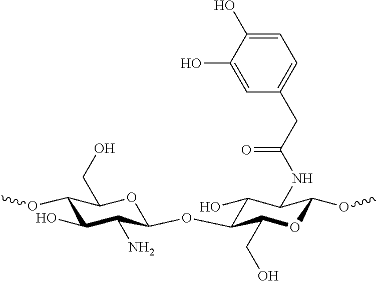 |

In some embodiments, the polymer precursor comprises a single type of polymer precursor. In certain embodiments, the polymer precursor comprises a combination of polymer precursors. In certain embodiments, the polymer precursor comprises a combination of polymer precursors listed in Tables 1 and 2. In some embodiments, the combination of polymer precursors consists of two different polymer precursors. In some embodiments, the combination of polymer precursors consists of three different polymer precursors. In some embodiments, the combination of polymer precursors consists of four different polymer precursors.

In some embodiments, the composition comprises an amount of polymer precursor to (polymer precursor, oxygen source, and permeation enhancer) of 40% to 90%. In some embodiments, the polymer precursor is dopamine (which may be provided in the form of dopamine HCl or another pharmaceutically acceptable salt of dopamine). In some embodiments, the composition comprises about 0.001 to about 1000 mg/mL of dopamine. In some embodiments, the composition comprises about 0.001 to about 500 mg/mL of dopamine. In some embodiments, the composition comprises about 0.01 to about 100 mg/mL of dopamine. In some embodiments, the composition comprises about 1 to about 100 mg/ml of dopamine. In some embodiments, the composition comprises about 1 to about 50 mg/mL of dopamine. In some embodiments, the composition comprises about 10 to about 40 mg/mL of dopamine. In some embodiments, the composition comprises about 1 to about 20 mg/mL of dopamine. In some embodiments, the composition comprises about 60 mg/mL of dopamine. In some embodiments, the composition comprises about 50 mg/mL of dopamine. In some embodiments, the composition comprises about 40 mg/mL of dopamine. In some embodiments, the composition comprises about 30 mg/mL of dopamine. In some embodiments, the composition comprises about 20 mg/mL of dopamine. In some embodiments, the composition comprises about 10 mg/mL of dopamine. In some embodiments, the composition comprises about 9.8 mg/mL of dopamine. In any of these embodiments, a pharmaceutically acceptable salt of dopamine can be used, such as dopamine hydrochloride. In any of these embodiments, polydopamine, a mixture of dopamine and polydopamine, or a pharmaceutically acceptable salt thereof can be used in place of dopamine or a pharmaceutically acceptable salt of dopamine. For example, in some embodiments, the composition comprises about 0.001 to about 1000 mg/ml of polydopamine. In some embodiments, the composition comprises about 0.001 to about 500 mg/mL of polydopamine. In some embodiments, the composition comprises about 0.01 to about 100 mg/mL of polydopamine. In some embodiments, the composition comprises about 1 to about 100 mg/mL of polydopamine. In some embodiments, the composition comprises about 1 to about 50 mg/mL of polydopamine. In some embodiments, the composition comprises about 10 to about 40 mg/mL of polydopamine. In some embodiments, the composition comprises about 1 to about 20 mg/mL of polydopamine. In some embodiments, the composition comprises about 60 mg/mL of polydopamine. In some embodiments, the composition comprises about 50 mg/mL of polydopamine. In some embodiments, the composition comprises about 40 mg/mL of polydopamine. In some embodiments, the composition comprises about 30 mg/mL of polydopamine. In some embodiments, the composition comprises about 20 mg/mL of polydopamine. In some embodiments, the composition comprises about 10 mg/mL of polydopamine. In some embodiments, the composition comprises about 9.8 mg/mL of polydopamine. In some embodiments, the composition comprises about 0.001 to about 1000 mg/mL of a mixture of dopamine and polydopamine. In some embodiments, the composition comprises about 0.001 to about 500 mg/mL of a mixture of dopamine and polydopamine. In some embodiments, the composition comprises about 0.01 to about 100 mg/mL of a mixture of dopamine and polydopamine. In some embodiments, the composition comprises about 1 to about 100 mg/mL of a mixture of dopamine and polydopamine. In some embodiments, the composition comprises about 1 to about 50 mg/mL of a mixture of dopamine and polydopamine. In some embodiments, the composition comprises about 10 to about 40 mg/mL of a mixture of dopamine and polydopamine. In some embodiments, the composition comprises about 1 to about 20 mg/mL of a mixture of dopamine and polydopamine. In some embodiments, the composition comprises about 60 mg/mL of a mixture of dopamine and polydopamine. In some embodiments, the composition comprises about 50 mg/mL of a mixture of dopamine and polydopamine. In some embodiments, the composition comprises about 40 mg/mL of a mixture of dopamine and polydopamine. In some embodiments, the composition comprises about 30 mg/mL of a mixture of dopamine and polydopamine. In some embodiments, the composition comprises about 20 mg/mL of a mixture of dopamine and polydopamine. In some embodiments, the composition comprises about 10 mg/mL of a mixture of dopamine and polydopamine. In some embodiments, the composition comprises about 9.8 mg/ml of a mixture of dopamine and polydopamine. When a mixture of dopamine and polydopamine is used, the amount of dopamine to polydopamine by weight can range from about 0.05:1 dopamine:polydopamine to about 1:0.05 dopamine:polydopamine on a weight/weight basis, such as about 0.1:1, about 0.2:1, about 0.25:1, about 0.3:1, about 0.4:1, about 0.5:1, about 0.6:1, about 0.7:1, about 0.75:1, about 0.8:1, about 0.9:1, about 1:1, about 1:0.9, about 1:0.8, about 1:0.75, about 1:0.7, about 1:0.6, about 1:0.5, about 1:0.4, about 1:0.3, about 1:0.25, about 1:0.2, or about 1:0.1 dopamine:polydopamine.

International Patent Application Nos. WO 2021/119350 and WO 2021/119354, which are incorporated herein by reference in their entirety, describe methods of forming polymers in situ in a subject.

### Oxygen Sources for Gastrointestinal Synthetic Epithelial Lining Precursor Compositions

In one aspect, the disclosure provides for a composition comprising a polymer precursor, an oxygen source, and a permeation enhancer. In some embodiments, the oxygen source is hydrogen peroxide or urea hydrogen peroxide. In some embodiments, the oxygen source is hydrogen peroxide. In some embodiments, the oxygen source is urea hydrogen peroxide. When the oxygen source contacts a catalyst endogenous to the subject, such as a catalase or a peroxidase, oxygen is liberated from the oxygen source, resulting in polymerization of the polymer precursor.

In some embodiments, the endogenous catalyst is selected from catalases or peroxidases. In some embodiments, the endogenous catalyst is a peroxidase. In certain embodiments, the peroxidase is eosinophil peroxidase, lactoperoxidase, or myeloperoxidase.

In some embodiments, the endogenous catalyst is a catalase. In some embodiments, the catalase is a bacterial catalase. In some embodiments, the catalase is a human catalase.

In some embodiments, the endogenous catalyst is located in the gastrointestinal (GI) tract of the subject. In some embodiments, the endogenous catalyst is located in the small intestine of the subject. In some embodiments, the endogenous catalyst is located in the duodenum of the subject.

In some embodiments, the endogenous catalyst is located in the upper GI of the subject. In some embodiments, the endogenous catalyst is located in the stomach of the subject.

In some embodiments, the composition comprises an amount of oxygen source to (polymer precursor, oxygen source, and permeation enhancer) of 1% to 15%.

In some embodiments, the composition comprises about 0.01 to about 100 mM of the oxygen source. In some embodiments, the composition comprises about 0.1 to about 50 mM of the oxygen source. In some embodiments, the composition comprises about 1 to about 30 mM of the oxygen source. In some embodiments, the composition comprises about 20 mM of the oxygen source. In some embodiments, the composition comprises a concentration of oxygen source compatible with ingestion by the subject.

### Permeation Enhancers for Gastrointestinal Synthetic Epithelial Linings (GSELs)

The current disclosure describes permeation enhancers for Gastrointestinal Synthetic Epithelial Linings (GSELs), which promote penetration of active pharmaceutical ingredients (APIs) across the gastrointestinal mucosa. GSELs localize an API in a specific location, resulting in prolonged contact with the gastrointestinal mucosa. However, some APIs may not permeate through the gastrointestinal barrier in sufficient amounts to be therapeutically effective, or may not permeate at all, despite the prolonged contact afforded by a GSEL. Permeation enhancers can be used in conjunction with a GSEL to increase the penetration of an API across the gastrointestinal mucosa.

Permeation enhancers may enhance paracellular transport. Paracellular transport is the passive transport of substances or molecules (e.g. active pharmaceutical ingredient) between adjacent epithelial cells. Permeation enhancers that improve paracellular transport may function, for example, by increasing the permeability of tight junctions. In some embodiments, the permeation enhancer enhances paracellular transport. Permeation enhancers may enhance transcellular transport. Transcellular transport is an active process, where a substance or molecule (e.g. a active pharmaceutical ingredient) is transported through a cell using a cellular transport mechanism. In some embodiments, the permeation enhancer enhances transcellular transport. In some embodiments, the permeation enhancer enhances both paracellular and transcellular transport.

In some embodiments, the composition comprises a polymer precursor, an oxygen source, and a permeation enhancer. In some embodiments, the composition comprises a polymer precursor, an oxygen source, and one or more permeation enhancers.

In some embodiments, the composition comprises an amount of one or more permeation enhancers to (polymer precursor, oxygen source, and one or more permeation enhancers) of 0.1% to 60%. In some embodiments, the composition comprises an amount of one or more permeation enhancers to (polymer precursor, oxygen source, and one or more permeation enhancers) of about 0.1% to about 50%, about 0.1% to about 40%, about 0.1% to about 30%, about 0.1% to about 20%, about 0.1% to about 10%, about 1% to about 60%, about 10% to about 60%, about 20% to about 60%, about 30% to about 60%, about 40% to about 60%, or about 50% to about 60%.

In some embodiments, ammonium salts can be used as permeation enhancers. These salts include, but are not limited to, ammonium carbonate (NHCO), ammonium sulfate (NHSO), ammonium citrate (NHCA), ammonium phosphate (NHPO), diammonium phosphate (DAP), monoammonium phosphate (MAP), ammonium bicarbonate (NHHCO), or ammonium chloride (NHCl). Ammonium lactate, ammonium acetate, ammonium sulfate, ammonium sulfite, triammonium citrate, ammonium propionate, and ammonium sulfamate can also be used. One particularly useful ammonium salt is ammonium carbonate. Another particularly useful ammonium salt is ammonium bicarbonate. Abbreviations used herein, such as NHCO to indicate ammonium carbonate, are not intended to designate chemical formulas, as the chemical formula for ammonium carbonate is (NH₄)₂CO₃.

In some embodiments, carbonate salts can be used as permeation enhancers. These salts include, but are not limited to, sodium carbonate (NaCO) or potassium carbonate (KCO). As previously noted, these are abbreviations, not chemical formulas.

In some embodiments, bicarbonate salts can be used as permeation enhancers, which include, but are not limited to, sodium bicarbonate (NaHCO) or potassium bicarbonate (KHCO). As previously noted, these are abbreviations, not chemical formulas.

In some embodiments, the permeation enhancer is an endogenous secretion. In some embodiments, the endogenous secretion is a bile salt. In some embodiments, the endogenous secretion is a bile acid.

In some embodiments, a bile salt or a bile acid can be used as a permeation enhancer. The bile salt or bile acid can comprise one or more bile salts, one or more bile acids, or any number of bile salts and/or bile acids in combination. Liquid GSELs are formulated in aqueous solution, and depending on the pH of the formulation, addition of a single bile acid, or a single bile salt, can result in a mixture of the bile acid and its bile salt (i.e., the conjugate base of the bile acid). For example, the pKa of cholic acid is given as 4.98 (see URL pubchem.ncbi.nlm.nih.gov/compound/Cholic-acid, referencing Serjeant EP, Dempsey B; Ionisation constants of organic acids in aqueous solution. IUPAC Chem Data Ser No. 23. NY,NY: Pergamon pp. 989 (1979)). If cholic acid is added to a formulation, and the pH raised to approximately 8, most of the material will be in the form of the cholate anion. Conversely, if a cholate salt is added to a formulation, and the pH lowered to approximately 2, most of the material will be in the form of cholic acid. Accordingly, when a bile salt or bile acid is recited, it is understood that the form of the material may change depending on the pH—that is, a bile salt may convert partially or mostly to a bile acid, and/or a bile acid may convert partially or mostly to a bile salt, depending on the pH of the formulation containing the bile salt or bile acid. A typical pH value for GSEL formulations is about 8.5, plus or minus 1 pH unit.

Bile acids for use as permeation enhancers include, but are not limited to, cholic acid (CHA), glycocholic acid (GCA), deoxycholic acid (DCA), glychochenodeoxychlic acid (GCDCA), glycodeoxycholic acid (GDCA), taurodeoxycholic acid (TDCA), taurocholic acid (TCA), bovine bile (OX), chenodeoxycholic acid (CDCA), taurochenodeoxycholic acid (TCDCA), lithocholic acid (LCA), glycolithocholate (GLC), glycohyocholate (GHC), taurolithocholate (TLC), ursodeoxycholic acid (UDCA), tauroursodeoxycholic acid (TUDCA), glycoursodeoxycholic acid (GUDCA), 12-monoketocholic acid (12-MKC), 7-monoketocholic acid (7-MKC), 7,12-diketocholic acid (7,12-DKC), 3,7,12-triketocholic acid (3,7,12-TKC), 12-monoketodeoxycholic acid (12-MKDC), taurodihydrofusidic acid, or 3-[(3-cholamidopropyl)dimethylammonio]-1-propanesulfonate (CHAPS). A particularly useful bile acid is glycocholic acid.

Bile salts for use as permeation enhancers include, but are not limited to, a salt of cholic acid, a salt of glycocholic acid, a salt of deoxycholic acid, a salt of glychochenodeoxychlic acid, a salt of glycodeoxycholic acid, a salt of taurodeoxycholic acid, a salt of taurocholic acid, a salt of bovine bile, a salt of chenodeoxycholic acid, a salt of taurochenodeoxycholic acid, a salt of lithocholic acid, a salt of glycolithocholate, a salt of glycohyocholate, a salt of taurolithocholate, a salt of ursodeoxycholic acid, a salt of tauroursodeoxycholic acid, a salt of glycoursodeoxycholic acid, a salt of 12-monoketocholic acid, a salt of 7-monoketocholic acid, a salt of 7,12-diketocholic acid, a salt of 3,7,12-triketocholic acid, a salt of 12-monoketodeoxycholic acid, a salt of taurodihydrofusidic acid, or a salt of 3-[(3-cholamidopropyl)dimethylammonio]-1-propanesulfonate. A particularly useful bile salt is a salt of glycocholic acid.

The bile salts can be sodium salts, including, but not limited to, sodium cholate, sodium glycocholate, sodium deoxycholate, sodium glychochenodeoxychlate, sodium glycodeoxycholate, sodium taurodeoxycholate, sodium taurocholate, a sodium salt of bovine bile, sodium chenodeoxycholate, sodium taurochenodeoxycholate, sodium lithocholate, sodium glycolithocholate, sodium glycohyocholate, sodium taurolithocholate, sodium ursodeoxycholate, sodium tauroursodeoxycholate, sodium glycoursodeoxycholate, sodium 12-monoketocholate, sodium 7-monoketocholate, sodium 7,12-diketocholate, sodium 3,7,12-triketocholate, sodium 12-monoketodeoxycholate, sodium taurodihydrofusidate, or sodium 3-[(3-cholamidopropyl)dimethylammonio]-1-propanesulfonate. A particularly useful sodium salt of a bile acid is sodium glycocholate.

In some embodiments, the permeation enhancer is a carnitine. In some embodiments, the carnitine is an acyl carnitine. In some embodiments, the carnitine is selected from the group consisting of lauroylcarnitine, palmitoylcarnitine, and palmitoyl carnitine chloride (PCC). In some embodiments, the carnitine is lauroylcarnitine. In some embodiments, the carnitine is palmitoylcarnitine.

In some embodiments, the permeation enhancer is a choline. In some embodiments, the choline is lysophosphatidyl choline.

In some embodiments, the permeation enhancer is an aromatic alcohol. In some embodiments, the aromatic alcohol is selected from the group consisting of benzyl alcohol, phenyl alcohol, phenoxyethanol, propyl gallate, butylated hydroxytoluene, and butylated hydroxyanisole. In some embodiments, the aromatic alcohol is benzyl alcohol, phenyl alcohol, or phenoxyethanol.

In some embodiments, the permeation enhancer is a piperazine derivative. In some embodiments, the piperazine derivative is selected from the group consisting of 1-phenylpiperazine, 1-methyl-4-phenylpiperazine, 1-(4-methylphenyl)piperazine, and 1-benzylpiperazine. In some embodiments, the piperazine derivative is 1-phenylpiperazine or 1-methyl-4-piperazine.

In some embodiments, the permeation enhancer is a mucoadhesive polymer. In some embodiments, the mucoadhesive polymer is selected from the group consisting of chitosan, chitosan hydrochloride, trimethylated chitosan chloride, and N,N,N-trimethyl chitosan chloride. In some embodiments, the mucoadhesive polymer is trimethylated chitosan chloride.

In some embodiments, the permeation enhancer is a cell penetrating peptide. In some embodiments, the cell penetrating peptide is selected from the group consisting of transportan, penetratin, oligoarginines, polyarginines, oligolysines, polylysines, oligotryptophans, and polytryptophans.

In some embodiments, the permeation enhancer is an amino acid. In some embodiments, the amino acid is tryptophan.

In some embodiments, the permeation enhancer is an ionic liquid. In some embodiments, the ionic liquid is selected from the group consisting of choline geranate, nicotinic acid and trigonelline. In some embodiments, the ionic liquid is choline geranate.

In some embodiments, the permeation enhancer is an organic solvent. In some embodiments, the solvent is selected from the group consisting of ethanol, 2-propanol, 1-propanol, 2-methyl-2-propanol, dimethyl sulfoxide, ethyl acetate, and acetone.

In some embodiments, the permeation enhancer is an anionic surfactant. In some embodiments, the anionic surfactant is sodium cholate or sodium dodecyl sulphate. In some embodiments, the anionic surfactant is sodium cholate. In some embodiments, the anionic surfactant is sodium dodecyl sulphate. Where chemically possible and pharmaceutically desirable, alternative pharmaceutically acceptable salts or the free acids of the foregoing compounds can be used.

In some embodiments, the permeation enhancer is a chelating agent. In some embodiments, the chelating agent is selected from the group consisting of EDTA, EGTA, and DTPA. In some embodiments, the chelating agent is EDTA.

In some embodiments, the permeation enhancer is a non-ionic surfactant. In some embodiments, the non-ionic surfactant is an ethoxylate. In some embodiments, the non-ionic surfactant is an alcohol ethoxylate (C_{X}E_{Y}, where X is the alcohol carbon number and Y is the ethylene oxide number).

In some embodiments, the non-ionic surfactant is a medium or long chain fatty acid sugar ester. In some embodiments, the medium or long chain fatty acid sugar ester is sodium laurate. In some embodiments, the non-ionic surfactant is a medium or long chain fatty acid sucrose ester. In some embodiments, the non-ionic surfactant is an ethoxylated fatty acid sugar ester. In some embodiments, the non-ionic surfactant is an ethoxylated sorbitan ester. In some embodiments, the non-ionic surfactant is an ethoxylated glyceride. In some embodiments, the non-ionic surfactant is selected from the group consisting of macrogol-8 glyceride, sucrose esters (e.g. sucrose laurate), ethoxylates, alkyl maltosides (e.g. dodecyl maltoside), short chain polyethylene glycol, Brij® series (polyoxyethylene(10) oleyl ether, polyoxyethylene (23) lauryl ether, etc.), polysorbates (polysorbate series PS 20, PS 40, PS 60, PS 65, PS 80), and Triton X-100. In some embodiments, the non-ionic surfactant is caprylocaproyl polyoxyl-8 glyceride (LABRASOL®), poloxamers, polyoxylglycerides, polyethylene monostearate.

In some embodiments, the permeation enhancer is a non-ionic detergent. In some embodiments, the non-ionic detergent is sucrose monolaurate or n-Tetradecyl ß-D-maltopyranoside (TDM). In some embodiments, the non-ionic detergent is sucrose monolaurate. In some embodiments, the non-ionic detergent is n-Tetradecyl ß-D-maltopyranoside (TDM).

In some embodiments, the permeation enhancer is a fatty acid, a fatty acid salt, an ethoxylated fatty acid ester, a sugar fatty acid ester, or an ethoxylated sugar fatty acid ester. In some embodiments, the fatty acid salt is sodium caprate (C₁₀). In some embodiments, the fatty acid salt is sodium caprylate (C₈). In some embodiments, the fatty acid ester is nonaethylene glycol monododecyl ether (C12E9). In some embodiments, the sugar fatty acid ester is a fatty acid ester of a monosaccharide. In some embodiments, the sugar fatty acid ester is an ethoxylated fatty acid ester of a monosaccharide. In some embodiments, the sugar fatty acid ester is a fatty acid ester of sorbitan or glucose. In some embodiments, the sugar fatty acid ester is an ethoxylated fatty acid ester of sorbitan or glucose. In some embodiments, the sugar fatty acid ester is a fatty acid ester of a disaccharide. In some embodiments, the sugar fatty acid ester is an ethoxylated fatty acid ester of a disaccharide. In some embodiments, the sugar fatty acid ester is a fatty acid ester of sucrose or maltose. In some embodiments, the sugar fatty acid ester is an ethoxylated fatty acid ester of sucrose or maltose. In some embodiments, the fatty acid or fatty acid salt is selected from the group consisting of dodecylmaltoside, sodium dodecyl sulfate, nonaethylene glycol monododecyl ether (C₁₂E₉), sodium laurate (C₁₂), sodium nonanoate (C₉), sodium undecanoate (C₁₁), sodium undecylenate (C11:1), sodium oleate, linoleic acid, and sucrose monolaurate. Where chemically possible and pharmaceutically desirable, alternative pharmaceutically acceptable salts or the free acids of the foregoing compounds can be used.

In some embodiments, the permeation enhancer is an N-acylated acid. In some embodiments, the N-acylated acid is acetylsalicylic acid. In some embodiments, the N-acylated acid is sodium N-[8-(2-hydroxybenzoyl)amino]caprylate (SNAC). In some embodiments, the N-acylated acid is 8-(N-2-hydroxy-5-chloro-benzoyl)-amino-caprylic acid (5-CNAC). In some embodiments, the N-acylated acid is 4-[(4-chloro-2-hydroxy-benzoyl)amino]butanoic acid (4-CNAB). In some embodiments, the N-acylated acid is N-(10-[2-hydroxybenzoyl]-amino)decanoic acid (SNAD). In some embodiments, the N-acylated acid is monosodium N-(4-chlorosalicyloyl)-4-aminobutyrate (5-CNAB). In some embodiments, the N-acylated acid is N-[8-(2-hydroxy-4-methoxy)benzoyl]amino caprylic acid (4-MOAC). In some embodiments, the N-acylated acid is sodium N-[8-(2-hydroxybenzoyl)amino]caprylate (SNAC). Where chemically possible and pharmaceutically desirable, either pharmaceutically acceptable salts or the free acids of the foregoing compounds can be used.

In some embodiments, the permeation enhancer is a high molecular weight polymer. In some embodiments, the high molecular weight polymer is a polysaccharide. In some embodiments, the high molecular weight polymer is an antibacterial toxin. In some embodiments, the antibacterial toxin is selected from the group consisting of zonula occludens toxin analogs, viral protein 8 analogs, and Clostridium perfringens enterotoxin analogs. In some embodiments, the high molecular weight polymer is chitosan or carboxymethylcellulose.

In some embodiments, the permeation enhancer is a caprylocaproyl PEG 8 glyceride.

In some embodiments, the permeation enhancer is a sugar-based surfactant. In some embodiments, the sugar-based surfactant is dodecyl-ß-D-maltopyranoside (DDM).

In some embodiments, the permeation enhancer is glyceryl monocaprate.

In some embodiments, the permeation enhancer is urea.

In some embodiments, the permeation enhancer is sodium docusate. Where chemically possible and pharmaceutically desirable, alternative pharmaceutically acceptable salts or the free acid of the foregoing compound can be used.

In some embodiments, the permeation enhancer is citric acid. Where pharmaceutically desirable, pharmaceutically acceptable salts of the foregoing compound can be used.

Combinations of permeation enhancers can be used, including, but not limited to:
glycocholic acid and ammonium carbonate;
a salt of glycocholic acid and ammonium carbonate and
sodium glycocholate and ammonium carbonate.

### Exemplary Compositions for Gastrointestinal Synthetic Epithelial Linings

The compositions used to form the gastrointestinal synthetic epithelial linings can be provided in solid form or liquid form. Examples of such compositions are provided, but it is understood that a wide variety of formulations can be used, and the compositions for use in forming GSELs are not restricted by these examples.

### Liquid Formulations

Polymer precursor: the composition can comprise about 0.001 to about 1000 mg/mL, about 0.001 to about 500 mg/mL, about 0.01 to about 100 mg/mL, about 1 to about 50 mg/mL, about 10 to about 40 mg/mL, about 1 to about 20 mg/mL, about 10 mg/mL, or about 9.8 mg/mL of polymer precursor (such as dopamine, polydopamine, a mixture of dopamine and polydopamine, or pharmaceutically acceptable salts thereof). In some embodiments, the composition comprises 5.4 mg/ml polymer precursor. In some embodiments, the composition comprises 9.8 mg/ml polymer precursor. In some embodiment, the composition comprises about 13.7 mg/ml polymer precursor. In some embodiment, the composition comprises about 19.6 mg/ml polymer precursor. In some embodiment, the composition comprises about 25 mg/ml polymer precursor. In some embodiment, the composition comprises about 29.4 mg/ml polymer precursor. In some embodiment, the composition comprises about 50 mg/ml polymer precursor. In some embodiment, the composition comprises about 75 mg/ml polymer precursor. In some embodiments, the composition comprises between about 5 mg/ml to about 75 mg/ml polymer precursor, between about 5 mg/ml to about 25 mg/ml polymer precursor, between about 5 mg/ml to about 50 mg/ml polymer precursor, between about 25 mg/ml to about 50 mg/ml polymer precursor, between about 25 mg/ml to about 75 mg/ml polymer precursor, or between about 50 mg/ml to about 75 mg/ml polymer precursor.

Oxygen source: the composition can comprise about 0.01 to about 100 mM, about 0.1 to about 50 mM, about 1 to about 30 mM, about 20 mM, or about 19.7 mM of the oxygen source; or the composition can comprise a concentration of oxygen source compatible with ingestion by the subject. In some embodiments, the composition comprises between about 0.1 mM to about 10 mM, between about 1 mM to about 25 mM, between about 1 mM to about 50 mM, between about 10 mM to about 25 mM, between about 10 mM to about 50 mM, between about 25 mM to about 50 mM, between about 25 mM to about 75 mM, between about 25 mM to about 100 mM, between about 50 mM to about 75 mM, between about 50 mM to about 100 mM, or between about 75 mM to about 100 mM of the oxygen source.

Permeation enhancer: the composition comprises an amount of permeation enhancer to (polymer precursor, oxygen source, and permeation enhancer) of about 0.1% to about 60%, about 0.1% to about 50%, about 0.1% to about 40%, about 0.1% to about 30%, about 0.1% to about 20%, about 0.1% to about 10%, about 1% to about 60%, about 10% to about 60%, about 20% to about 60%, about 30% to about 60%, about 40% to about 60%, or about 50% to about 60%. In some embodiments, the amount of permeation enhancer is about 40 mg/ml, about 50 mg/ml, about 60 mg/ml, about 70 mg/ml to about 80 mg/ml. In some embodiments, the amount of permeation enhancer is 40 mg/ml. In some embodiments, the amount of permeation enhancer is 80 mg/ml. In some embodiments, the amount of permeation enhancer is between about 40 mg/ml and about 80 mg/ml. In some embodiments, the amount of permeation enhancer is between about 40 mg/ml and about 50 mg/ml, between about 40 mg/ml and about 60 mg/ml, between about 40 mg/ml and about 70 mg/ml, between about 50 mg/ml and about 60 mg/ml, between about 50 mg/ml and about 70 mg/ml, between about 50 mg/ml and about 80 mg/ml, between about 60 mg/ml and about 70 mg/ml, between about 60 mg/ml and about 80 mg/ml, or between 70 mg/ml and about 80 mg/ml.

API: the active pharmaceutical ingredient can comprise an amount of API to (polymer precursor, oxygen source, permeation enhancer, and API) of about 0.01% to about 10%, about 0.1 to about 10%, about 1% to about 10%, about 5% to about 10%, about 0.1% to about 5%, or about 1% to about 5%. In some embodiments, the amount of API is between about 0.5 mg/ml to 25 mg/ml. In some embodiments, the amount of API is between about 0.67 mg/ml to about 15 mg/ml, between about 1 mg/ml to about 10 mg/ml, between about 1 mg/ml to about 15 mg/ml, between about 1 mg/ml to about 20 mg/ml, between about 1 mg/ml to about 25 mg/ml, between about 5 mg/ml to about 10 mg/ml, between about 5 mg/ml to about 15 mg/ml, between about 5 mg/ml to about 20 mg/ml, between about 5 mg/ml to about 25 mg/ml, between about 10 mg/ml to about 15 mg/ml, between about 10 mg/ml to about 20 mg/ml, between about 10 mg/ml to about 25 mg/ml, between about 15 mg/ml to about 20 mg/ml, between about 15 mg/ml to about 25 mg/ml, between about 20 mg/ml to about 25 mg/ml, about 16 mg/ml, about 17 mg/ml, about 18 mg/ml, about 19 mg/ml, or about 20 mg/ml. In some embodiments, the amount of API is 0.67 mg/ml. In some embodiments the amount of API is 15 mg/ml. In some embodiments, the amount of API is about 20 mg/ml. In some embodiments, the active pharmaceutical ingredient is semaglutide.

Optionally, a viscosity modifier can be added, such as xanthan gum, hydroxypropylmethyl cellulose, methyl cellulose, carboxymethyl cellulose, chitosan, hydroxyethyl cellulose, or sodium alginate. Viscosity can be adjusted to between about 0.01 to about 50 Pascal-seconds at 0.1 per second shear rates.

Buffers can be added to maintain appropriate pH of the liquid formulation, for example, between about pH 7 and about pH 10, such as pH 7.4.

A particular formulation falling within these ranges is:
9.8 mg/mL dopamine hydrochloride and 25 mg/mL polydopamine as polymer precursors (34.9 mg/mL polymer precursor);
0.67 mg/mL hydrogen peroxide (19.7 mM) as oxygen source;
20 mg/mL sodium caprate as permeation enhancer (sodium caprate makes up 36% of the weight of dopamine hydrochloride, polydopamine, hydrogen peroxide, sodium caprate);
0.67 mg/mL semaglutide (API) (the API makes up 1.2% of the amount of dopamine hydrochloride, polydopamine, hydrogen peroxide, sodium caprate, and semaglutide); and 50 mM Tris buffer.

Another particular formulation falling within these ranges is: 9.8 mg/mL dopamine hydrochloride and 25 mg/mL polydopamine as polymer precursors (34.9 mg/mL polymer precursor);
0.67 mg/mL hydrogen peroxide (19.7 mM) as oxygen source;
20 mg/mL sodium glycocholate and ammonium carbonate as permeation enhancer (makes up 36% of the weight of dopamine hydrochloride, polydopamine, hydrogen peroxide, sodium glycocholate, and ammonium carbonate);
0.67 mg/mL semaglutide (API) (the API makes up 1.2% of the amount of dopamine hydrochloride, polydopamine, hydrogen peroxide, sodium glycocholate, ammonium carbonate, and semaglutide); and 50 mM Tris buffer.

### Solid Formulations

Polymer precursor: the composition can comprise about 10% to about 75%, about 20% to about 75%, about 30% to about 75%, about 40% to about 75%, or about 50% to about 75% of polymer precursor (such as dopamine, polydopamine, a mixture of dopamine and polydopamine, or pharmaceutically acceptable salts thereof).

Oxygen source: the composition can comprise about 1% to about 15%, about 1% to about 10%, about 1% to about 5%, about 5% to about 15%, or about 10% to about 15% of the oxygen source; or the composition can comprise an amount of oxygen source compatible with ingestion by the subject.

Permeation enhancer: the composition comprises an amount of permeation enhancer to (polymer precursor, oxygen source, and permeation enhancer) of about 0.1% to about 60%, about 0.1% to about 50%, about 0.1% to about 40%, about 0.1% to about 30%, about 0.1% to about 20%, about 0.1% to about 10%, about 1% to about 60%, about 10% to about 60%, about 20% to about 60%, about 30% to about 60%, about 40% to about 60%, or about 50% to about 60%.

API: the active pharmaceutical ingredient can comprise an amount of API to (polymer precursor, oxygen source, permeation enhancer, and API) of about 0.01% to about 10%, about 0.1 to about 10%, about 1% to about 10%, about 5% to about 10%, about 0.1% to about 5%, or about 1% to about 5%.

Optionally, xanthan gum, hydroxypropylmethyl cellulose, methyl cellulose, carboxymethyl cellulose, chitosan, hydroxyethyl cellulose, or sodium alginate can be added to the solid dosage formulation, in order to increase viscosity when the formulation solubilizes in the digestive tract.

Solid buffers can be used in order to control pH when the formulation solubilizes in the digestive tract.

A particular solid formulation falling within these ranges is:
18.8 wt % dopamine hydrochloride and 47.95 wt % polydopamine (66.75% polymer precursor);
3.54 wt % urea hydrogen peroxide;
16.58 wt % sodium caprylate (sodium caprate makes up 19% of the weight of dopamine hydrochloride, polydopamine, urea hydrogen peroxide, and sodium caprate);
0.52 wt % methotrexate (methotrexate makes up 0.6% of the weight of dopamine hydrochloride, polydopamine, urea hydrogen peroxide, sodium caprate, and methotrexate);
4.30 wt % Tris base; and
8.31 wt % Tris hydrochloride.

A particular solid formulation falling within these ranges is:
18.8 wt % dopamine hydrochloride and 47.95 wt % polydopamine (66.75% polymer precursor);
3.54 wt % urea hydrogen peroxide;
16.58 wt % sodium caprylate (sodium caprylate makes up 19% of the weight of dopamine hydrochloride, polydopamine, urea hydrogen peroxide, and sodium caprylate);
0.52 wt % methotrexate (methotrexate makes up 0.6% of the weight of dopamine hydrochloride, polydopamine, urea hydrogen peroxide, sodium caprate, and methotrexate);
4.30 wt % Tris base; and
8.31 wt % Tris hydrochloride.

A particular solid formulation falling within these ranges is:
18.8 wt % dopamine hydrochloride and 47.95 wt % polydopamine (66.75% polymer precursor);
3.54 wt % urea hydrogen peroxide;
16.58 wt % sodium caprate (sodium caprate makes up 19% of the weight of dopamine hydrochloride, polydopamine, urea hydrogen peroxide, and sodium caprate);
0.52 wt % methotrexate (methotrexate makes up 0.6% of the weight of dopamine hydrochloride, polydopamine, urea hydrogen peroxide, sodium caprate, and methotrexate);
4.30 wt % Tris base; and
8.31 wt % Tris hydrochloride.

A particular solid formulation falling within these ranges is:
18.8 wt % dopamine hydrochloride and 47.95 wt % polydopamine (66.75% polymer precursor);
3.54 wt % urea hydrogen peroxide;
16.58 wt % sodium glycocholate, and XX % ammonium carbonate (sodium glycocholate and ammonium carbonate makes up 19% of the weight of dopamine hydrochloride, polydopamine, urea hydrogen peroxide, sodium glycocholate, and ammonium carbonate);
0.52 wt % methotrexate (methotrexate makes up 0.6% of the weight of dopamine hydrochloride, polydopamine, urea hydrogen peroxide, sodium caprate, and methotrexate);
4.30 wt % Tris base; and
8.31 wt % Tris hydrochloride.

Where chemically possible and pharmaceutically desirable, alternative pharmaceutically acceptable salts or the free acids or free bases of any of the foregoing compounds can be used.

### Evaluation of Permeation Enhancers

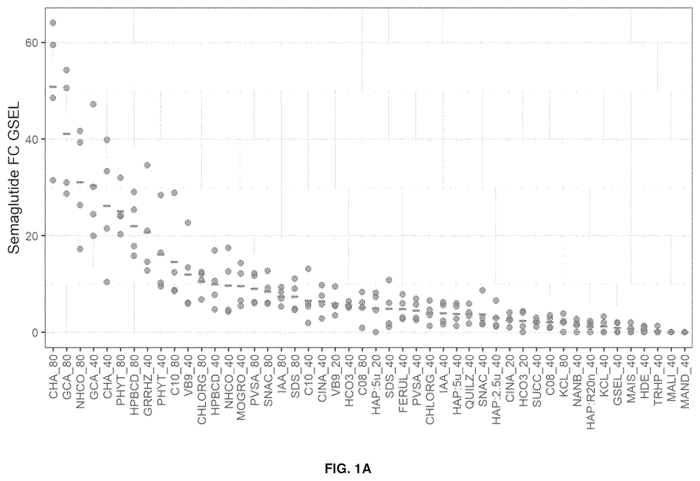

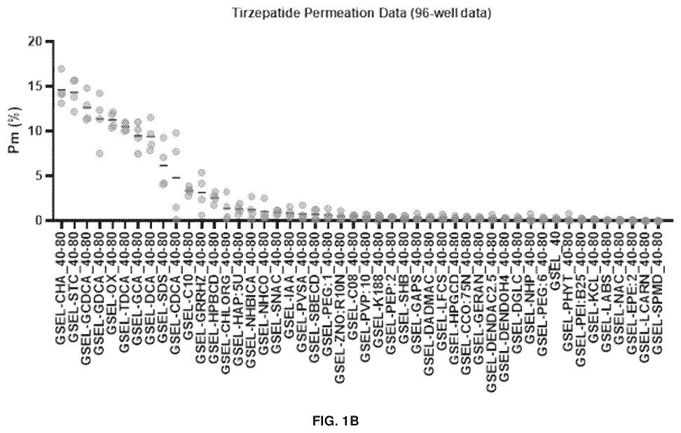

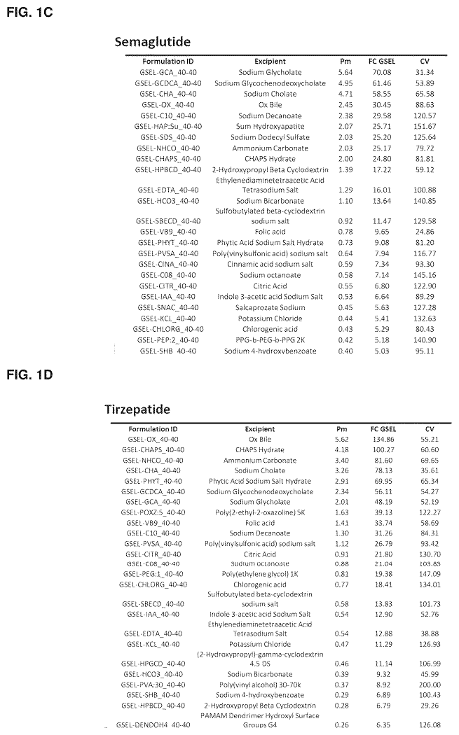

Various assays to measure the absorption of APIs across the gastrointestinal tract are well-known, and can be adapted to evaluate the ability of permeation enhancers to enhance permeation of active pharmaceutical ingredients using a gastrointestinal synthetic epithelial lining (GSEL). The Franz cell is often used for such assays. A Franz cell has an upper chamber, called a donor compartment or donor chamber, and a lower chamber, called a receiving compartment or receiving chamber. An appropriate solution is added to each compartment, such as phosphate-buffered saline (PBS). The receiving compartment a sample port to withdraw aliquots for analysis, and typically also a magnetic stir bar or other mechanism for uniform mixing of the receiving compartment solution. The receiving compartment can be jacketed by a water bath to maintain a constant temperature. The two compartments are separated by the barrier to be tested, which is clamped by a joint formed by the donor compartment and the receiving compartment. An illustration of a Franz cell is found in FIG. 1B of Pulsoni et al., “Comparison Between Franz Diffusion Cell and a novel Micro-physiological System for In Vitro Penetration Assay Using Different Skin Models,” SLAS Technology 27(3): 161 (2022), doi.org/10.1016/j.slast.2021.12.006.

For testing the permeation enhancers for GSELs, a section of intestinal tissue is obtained, such as from a pig or from a human cadaver, in accordance with all applicable legal and ethical rules. A GSEL can be deposited on the intestinal tissue before the tissue is placed in the Franz cell. Alternatively, the intestinal tissue can be placed in the Franz cell, and a GSEL can be deposited on the tissue as it resides in the Franz cell. The tissue can be rinsed to remove excess GSEL solution, and permeation of an active pharmaceutical ingredient (API) across the GSEL-coated tissue can be measured. Controls can include tissue without a GSEL, and tissue with a GSEL lacking the permeation enhancer. Tests of permeation enhancer can include a system with intestinal tissue having a GSEL that comprises a permeation enhancer, where the tissue is deployed in the Franz cell, and where an API is subsequently added to the donor compartment (for example, added to the solution in the donor compartment); permeation of the API into the receiving compartment is then measured at various time points to determine the amount of API that traverses the intestinal tissue into the receiving compartment. Alternatively, a test of permeation enhancer in a GSEL can include a system with intestinal tissue having a GSEL that comprises a permeation enhancer and the API, such that the API is contained in the GSEL and is in close association with the intestinal tissue as a component of the GSEL. Again, permeation of the API into the receiving compartment is then measured at various time points to determine the amount of API that traverses the intestinal tissue into the receiving compartment. Typically, a minimal volume is withdrawn and replaced at each time point. Comparison of the amount of permeation across the GSEL with and without permeation enhancer indicates the effectiveness of a particular permeation enhancer for a particular API. Further details of these comparison experiments for evaluation of permeation enhancers are described in the Example section herein.

Substitute or proxy molecules, such as fluorescein isothiocyanate-dextran (FITC-dextran) of varying molecular weight, can be used as test molecules for permeation enhancers. For example, FITC-dextran of 4,000 MW (FITC-dextran-4k) can be used in place of an API to test the efficacy of a permeation enhancer.

In some embodiments, the permeation enhancer in a GSEL increases the amount of permeation of an API across intestinal tissue by a factor of about 1.2 to about 5 at a given time point, as compared to the GSEL without the permeation enhancer, in a Franz cell using phosphate-buffered saline as donor compartment and receiving compartment liquid. In some embodiments, the permeation enhancer in a GSEL increases the amount of permeation of an API across intestinal tissue by a factor of up to about 1.5, up to about 2, up to about 3, up to about 4, or up to about 5 at a given time point, such as a factor or about 1.2 to about 5, about 1.5 to about 5, about 2 to about 5, about 3 to about 5, or about 4 to about 5, as compared to the GSEL without the permeation enhancer, in a Franz cell using phosphate-buffered saline as donor compartment and receiving compartment liquid. For example, if at a given timepoint, 1 mg of API was transported across intestinal tissue with a GSEL lacking permeation enhancer, and 1.5 mg of API was transported across intestinal tissue with a GSEL containing permeation enhancer, the amount of permeation was increased by a factor of 1.5.

In some embodiments, the permeation enhancer in a GSEL increases the total amount of permeation of an API across intestinal tissue by a factor of about 1.2 to about 50 over a 24-hour time period, as compared to the GSEL without the permeation enhancer, in a Franz cell using phosphate-buffered saline as donor compartment and receiving compartment liquid. Measurement of the total amount of API transported from the donor compartment to the receiving compartment over a given period of time can serve as an approximate in vitro model to provide an estimate of AUC₀₋₂₄ in an organism. In some embodiments, the permeation enhancer in a GSEL increases the total amount of permeation of an API across intestinal tissue by a factor of up to about 1.2, about 1.5, about 2, about 5, about 10, about 20, about 30, about 40, or about 50, such as about 1.2 to about 50, about 1.5 to 50, about 2 to 50, about 5 to 50, about 10 to 50, about 20 to 50, about 30 to 50, about 40 to 50, about 1.2 to 40, about 1.2 to 30, about 1.2 to 20, about 1.2 to 10, or about 1.2 to 5 over a 24-hour time period, as compared to the GSEL without the permeation enhancer, in a Franz cell using phosphate-buffered saline as donor compartment and receiving compartment liquid.

### Active Pharmaceutical Ingredients (APIs) for Administration Using Gastrointestinal Synthetic Epithelial Linings

In one aspect, the disclosure provides for a composition comprising an active pharmaceutical ingredient. In some embodiments, the composition comprises a polymer precursor, an oxygen source, a permeation enhancer, and an active pharmaceutical ingredient. In some embodiments, the oxygen source contacts a catalyst endogenous to the subject, resulting in polymerization of the polymer precursor. In some embodiments, the polymer precursor is polymerized into a polymer. In some embodiments, the active pharmaceutical ingredient is retained or encapsulated in the polymer. In some embodiments, the active pharmaceutical ingredient is retained on the polymer.

For any of the active pharmaceutical ingredients disclosed herein, the API or APIs can be combined into the GSEL composition for administration. For any of the active pharmaceutical ingredients disclosed herein, the API or APIs can be administered in a separate dosage form from the GSEL composition for administration. For any of the active pharmaceutical ingredients disclosed herein, the API or APIs can be combined into the GSEL composition for administration, and also administered in a separate dosage form from the GSEL composition for administration. When the API or APIs are administered in a separate dosage form from the GSEL composition for administration, they can be administered before administration of the GSEL composition, concurrently with the GSEL composition, or after administration of the GSEL composition.

In some embodiments, the composition comprises an amount of active pharmaceutical ingredient(s) to (polymer precursor, oxygen source, permeation enhancer, and active pharmaceutical ingredient(s)) of 0.1% to 10%.

In some embodiments, the active pharmaceutical ingredient is a statin.

In some embodiments, the active pharmaceutical ingredient treats obesity.

In some embodiments, the active pharmaceutical ingredient treats type 2 diabetes.

In some embodiments, the active pharmaceutical ingredient treats metabolic syndrome.

In some embodiments, the active pharmaceutical ingredient treats non-alcoholic fatty liver disease.

In some embodiments, the active pharmaceutical ingredient treats nonalcoholic steatohepatitis.

In some embodiments, the active pharmaceutical ingredient treats Crohn's disease.

In some embodiments, the active pharmaceutical ingredient treats an infectious disease. In some embodiments, the active pharmaceutical ingredient is an antibiotic drug. In some embodiments, the active pharmaceutical ingredient is an antiparasitic drug. In some embodiments, the active pharmaceutical ingredient is an anthelmintic drug. In some embodiments, the active pharmaceutical ingredient is an antiviral drug.

In some embodiments, the active pharmaceutical ingredient is a contraceptive.

### Small Organic Molecules

In some embodiments, the composition comprises an active pharmaceutical ingredient, wherein the active pharmaceutical ingredient is a small organic molecule. In some embodiments, the active pharmaceutical ingredient is a small molecule having a molecular weight of about 1 kD or less.

In certain embodiments, the active pharmaceutical ingredient is an antibiotic. Exemplary antibiotics include, but are not limited to, penicillins (e.g., penicillin, amoxicillin), cephalosporins (e.g., cephalexin), macrolides (e.g., crythromycin, clarithormycin, azithromycin, troleandomycin), fluoroquinolones (e.g., ciprofloxacin, levofloxacin, ofloxacin), sulfonamides (e.g., co-trimoxazole, trimethoprim), tetracyclines (e.g., tetracycline, chlortetracycline, oxytetracycline, demeclocycline, methacycline, sancycline, doxycline, aurcomycin, terramycin, minocycline, 6-deoxytetracycline, lymecycline, meclocycline, methacycline, rolitetracycline, and glycylcycline antibiotics (e.g., tigecycline)), aminoglycosides (e.g., gentamicin, tobramycin, paromomycin), aminocyclitol (e.g., spectinomycin), chloramphenicol, sparsomycin, quinupristin/dalfoprisin (Syndercid™). In certain embodiments, the antibiotic is a ribosome-targeting antibiotic.

### Macromolecular Active Pharmaceutical Ingredients

In some embodiments, the composition comprises an active pharmaceutical ingredient, wherein the active pharmaceutical ingredient is a macromolecule. In some embodiments, the composition comprises an active pharmaceutical ingredient, wherein the active pharmaceutical ingredient is a macromolecule having a molecular weight between about 1 kD and about 160 kD, such as about 1 kD to about 10 kD, or about 1 kD to about 5 kD. In some embodiments, the composition comprises an active pharmaceutical ingredient, wherein the active pharmaceutical ingredient is a macromolecule having a molecular weight between about 1 kD to about 125 kD, about 1 kD to about 100 kD, about 1 kD to about 75 kD, about 1 kD to about 50 kD, or about 1 kD to about 25 kD.

### Peptides and Proteins

In some embodiments, the composition comprises an active pharmaceutical ingredient, wherein the active pharmaceutical ingredient is a macromolecule, and the macromolecule is a polypeptide. In some embodiments, the polypeptide has a molecular weight of less than about 10 kD, at least about 1 kD, or between 1 kD and 10 kD. In some embodiments, the polypeptide comprises fewer than about 80 amino acids, at least about 8 amino acids, or between about 8 amino acids and about 80 amino acids. In some embodiments, the polypeptide comprises insulin, semaglutide, a GLP-1 receptor agonist, tirzepatide, liraglutide, desmopressin, octreotide, an analgesic peptide, difelikefalin, H-20, an antibiotic, cyclosporin, vancomycin, lactase, beta galactosidase, exenatide, teriparatide, nafarelin, buserelin, captopril, daptomycin, an antibody, caplacizumab, ozoralizumab, brolucizumab, ranibizumab, bevacizumab, trastuzumab, rituximab, adalimumab, an enzyme, lipase, a protease, phenylalanine hydroxylase, carbamoylphosphate synthetase I, glucose oxidase, or L-asparaginase.

### Nucleic Acids

In some embodiments, the composition comprises an active pharmaceutical ingredient, wherein the active pharmaceutical ingredient is a macromolecule, and the macromolecule is a polynucleotide. In some embodiments, the polynucleotide has a molecular weight of less than about 1.5 million, at least about 5 kD, or between about 5 kD and about 1500 kD, about 5 kD and about 1000 kD, about 5 kD and about 750 kD, about 5 kD and about 500 kD, about 5 kD and about 250 kD, about 5 kD and about 100 kD, about 5 kD and about 50 kD, about 5 kD and about 25 kD, or about 5 kD and about 10 kD. In some embodiments, the polynucleotide is single-stranded. In some embodiments, the polynucleotide is double-stranded. In some embodiments, the polynucleotide comprises fewer than about 5000 bases, at least about 10 bases, or between about 10 bases and about 5000 bases, about 10 bases and about 2500 bases, about 10 bases and about 1000 bases, about 10 bases and about 500 bases, about 10 bases and about 100 bases, about 10 bases and about 50 bases, or about 10 bases and about 25 bases. In some embodiments, the polynucleotide comprises fewer than about 2500 base pairs, at least about 5 base pairs, or between about 5 and about 2500 base pairs, about 5 and about 1000 base pairs, about 5 and about 500 base pairs, about 5 and about 250 base pairs, about 5 and about 100 base pairs, about 5 and about 50 base pairs, about 5 and about 25 base pairs, or about 5 and about 10 base pairs. In some embodiments, the polynucleotide comprises fewer than about 15 base pairs, at least about 5 base pairs, or between about 5 and about 15 base pairs. In some embodiments, the polynucleotide comprises an antisense oligonucleotide, mipomersen, patisiran, exondys, an siRNA, or an IBD targeting siRNA.

### Dosage Forms

The gastrointestinal synthetic epithelial lining (GSEL) compositions for formation of the GSEL are administered orally.

The composition and its uses described herein comprise administering to a subject an effective amount of a composition comprising a polymer precursor, an oxygen source, and a permeation enhancer that enhances permeation of one or more active pharmaceutical ingredients. In some embodiments, the composition further comprises an active pharmaceutical ingredient (API).

In certain aspects, further provided herein are compositions comprising dopamine, an oxygen source, a permeation enhancer, and optionally a buffer.

In some embodiments, the composition comprises a polymer precursor, an oxygen source, and a permeation enhancer that enhances uptake of one or more active pharmaceutical ingredient, and optionally, a buffer. In some embodiments, the buffer comprises phosphate, acetate, citrate, N-[tris(hydroxymethyl)methyl]glycine), (tris(hydroxymethyl)aminomethane), or (2-(bis(2-hydroxyethyl)amino)acetic acid). In some embodiments, the buffer comprises tris(hydroxymethyl)aminomethane.

In some embodiments, the composition further comprises an enzyme, a nutrient blocker, a radioprotective agent, a nutraceutical, a diagnostic agent, or a combination thereof. In some embodiments, the composition further comprises an enzyme. In some embodiments, the composition further comprises a radioprotective agent. In some embodiments, the composition further comprises an active pharmaceutical ingredient. In some embodiments, the composition further comprises a diagnostic agent. In some embodiments, the composition further comprises a combination of two or more of an enzyme, a nutraceutical, a radioprotective agent, an active pharmaceutical ingredient, and a diagnostic agent.

In some embodiments, the composition is administered orally. In some embodiments, the composition is a liquid or a solid dosage form. In some embodiments, the composition is in the form of a solution, a gel, a powder, a tablet, or a capsule.

In some embodiments, the composition is in a solution and has a pH of about 7 to about 10. In some embodiments, the composition is in a solution and has a pH of about 7 to about 9. In some embodiments, the composition is in a solution and has a pH of about 8.5. In some embodiments, the composition is in a solution and has a pH of about 7.4.

In certain embodiments, the subject to which the composition is administered is a human. In certain embodiments, the subject to which the composition is administered is an animal. In certain embodiments, the subject is a mammal. In certain embodiments, the subject is a non-human mammal. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate.

Compositions can be prepared, packaged, and/or sold in bulk, as a single unit dose, and/or as a plurality of single unit doses. A “unit dose” is a discrete amount of the composition comprising a predetermined amount of the agent or active ingredient. The amount of the agent or active ingredient is generally equal to the dosage of the agent or active ingredient which would be administered to a subject and/or a convenient fraction of such a dosage, such as one-half or one-third of such a dosage.

Compositions provided herein are typically formulated in dosage unit form for case of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific agent or active ingredient employed; the specific composition employed; the age, body weight, general health, sex, and diet of the subject; the time of administration, and rate of excretion of the specific agent or active ingredient employed; the duration of the treatment; drugs used in combination or coincidental with the specific agent or active ingredient employed; and like factors well known in the medical arts.

The compositions provided herein can be administered by enteral (e.g., oral) administration, or by feeding tube or gastric tube.

The exact amount of agent or agent or active ingredient required to achieve an effective amount will vary from subject to subject, depending, for example, on species, age, and general condition of a subject, severity of the side effects or disorder, identity of the particular agent or active ingredient, mode of administration, and the like. An effective amount may be included in a single dose (e.g., single oral dose) or multiple doses (e.g., multiple oral doses). In certain embodiments, when multiple doses are administered to a subject, any two doses of the multiple doses can include different amounts of an agent or active ingredient described herein, or substantially the same amounts of an agent or active ingredient described herein. In certain embodiments, when multiple doses are administered to a subject, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject is one dose per day. In certain embodiments, the frequency of administering the multiple doses to the subject is two doses per day. In certain embodiments, the frequency of administering the multiple doses to the subject is three doses per day. In certain embodiments, when multiple doses are administered to a subject, the duration between the first dose and last dose of the multiple doses can be one day, two days, four days, one week, two weeks, three weeks, one month, two months, three months, four months, six months, nine months, one year, two years, three years, four years, five years, seven years, ten years, fifteen years, twenty years, or over the lifetime of the subject. In certain embodiments, the duration between the first dose and last dose of the multiple doses is three months, six months, or one year. In certain embodiments, the duration between the first dose and last dose of the multiple doses is the lifetime of the subject.

The compositions as described herein can be administered in combination with one or more additional pharmaceutical agents (e.g., therapeutically active agents). The compositions can be administered in combination with additional pharmaceutical agents that improve their activity (e.g., activity (e.g., potency and/or efficacy) in treating a disease in a subject in need thereof, improve bioavailability, improve safety, reduce drug resistance, reduce and/or modify metabolism, inhibit excretion, and/or modify distribution in a subject.

The compositions can be administered concurrently with, prior to, or subsequent to one or more additional pharmaceutical agents, which may be useful as, e.g., combination therapies.

Any of the monomers, polymer precursors, prepolymers, permeation enhancers, and active pharmaceutical ingredients can be administered as a pharmaceutically acceptable salt where chemically possible and pharmaceutically desirable. Any of the monomers, polymer precursors, prepolymers, permeation enhancers, and active pharmaceutical ingredients described herein as a salt can alternatively be used as the free acid or free base where chemically possible and pharmaceutically desirable. Common pharmaceutically acceptable salts include hydrochloride, sodium, sulfate, acetate, phosphate, diphosphate, chloride, potassium, maleate, calcium, citrate, mesylate, nitrate, tartrate, aluminum, and gluconate salts. Additional pharmaceutically acceptable salts are enumerated in Gupta et al., Salts of Therapeutic Agents: Chemical, Physicochemical, and Biological Considerations. Molecules. 2018 Jul. 14; 23(7): 1719. doi: 10.3390/molecules23071719. PMID: 30011904; PMCID: PMC6100526; and in Berge et al., J Pharm Sci. 1977 January;66(1):1-19. doi: 10.1002/jps.2600660104. PMID: 833720.

### Enumerate Embodiments

The disclosure is further illustrated by the following enumerated embodiments. The various embodiments can be combined in any manner where practical.

Embodiment 1. A composition for oral administration for forming a polymer in situ in a subject, comprising:
a polymer precursor;
an oxygen source; and
a permeation enhancer that enhances permeation of one or more active pharmaceutical ingredients.

Embodiment 2. The composition of embodiment 1, further comprising one or more active pharmaceutical ingredients.

Embodiment 3. The composition of embodiment 1 or embodiment 2, further comprising a buffering agent.

Embodiment 4. The composition of any one of embodiments 1-3, further comprising one or more additional permeation enhancers.

Embodiment 5. The composition of any one of embodiments 1-4, wherein the composition comprises an amount of polymer precursor to (polymer precursor, oxygen source, and permeation enhancer) of 40% to 90%.

Embodiment 6. The composition of any one of embodiments 1-5, wherein the composition comprises an amount of oxygen source to (polymer precursor, oxygen source, and permeation enhancer) of 1% to 15%.

Embodiment 7. The composition of any one of embodiments 1-6, wherein the composition comprises an amount of permeation enhancer to (polymer precursor, oxygen source, and permeation enhancer) of 0.1% to 60%.

Embodiment 8. The composition of any one of embodiments 1-7, wherein the composition comprises an amount of polymer precursor to (polymer precursor, oxygen source, permeation enhancer, and active pharmaceutical ingredient(s)) of 40% to 90%.

Embodiment 9. The composition of any one of embodiments 1-8, wherein the composition comprises an amount of oxygen source to (polymer precursor, oxygen source, permeation enhancer, and active pharmaceutical ingredient(s)) of 1% to 15%.

Embodiment 10. The composition of any one of embodiments 1-9, wherein the composition comprises an amount of permeation enhancer to (polymer precursor, oxygen source, permeation enhancer, and active pharmaceutical ingredient(s)) of 1% to 60%.

Embodiment 11. The composition of any one of embodiments 1-10, wherein the composition comprises an amount of active pharmaceutical ingredient(s) to (polymer precursor, oxygen source, permeation enhancer, and active pharmaceutical ingredient(s)) of 0.1% to 10%.

Embodiment 12. The composition of any one of embodiments 2-11, wherein the active pharmaceutical ingredient is a macromolecule having a molecular weight between about 1 kD and about 160 kD.

Embodiment 13. The composition of embodiment 12, wherein the macromolecule is a polypeptide or polynucleotide.

Embodiment 14. The composition of embodiment 12, wherein the macromolecule is a polypeptide.

Embodiment 15. The composition of embodiment 14, wherein the polypeptide has a molecular weight of between about 1 kD and about 10 kD.

Embodiment 16. The composition of embodiment 14, wherein the polypeptide comprises between about 8 and about 80 amino acids.

Embodiment 17. The composition of embodiment 14, wherein the polypeptide comprises insulin, semaglutide, a GLP-1 receptor agonist, tirzepatide, liraglutide, desmopressin, octreotide, an analgesic peptide, difelikefalin, H-20, an antibiotic, cyclosporin, vancomycin, lactase, beta galactosidase, exenatide, teriparatide, nafarelin, buserelin, captopril, daptomycin, an antibody, caplacizumab, ozoralizumab, brolucizumab, ranibizumab, bevacizumab, trastuzumab, rituximab, adalimumab, an enzyme, lipase, a protease, phenylalanine hydroxylase, carbamoylphosphate synthetase I, glucose oxidase, or L-asparaginase.

Embodiment 18. The composition of embodiment 12, wherein the macromolecule is a polynucleotide.

Embodiment 19. The composition of embodiment 18, wherein the polynucleotide has a molecular weight of between about 5 kD and about 1500 kD.

Embodiment 20. The composition of embodiment 18 or embodiment 19, wherein the polynucleotide is single-stranded.

Embodiment 21. The composition of embodiment 18 or embodiment 19, wherein the polynucleotide is double-stranded.

Embodiment 22. The composition of embodiment 18, wherein the polynucleotide is single-stranded and comprises between about 10 and about 5000 bases.

Embodiment 23. The composition of embodiment 18, wherein the polynucleotide is single-stranded and comprises between about 10 and about 1000 bases.

Embodiment 24. The composition of embodiment 18, wherein the polynucleotide is single-stranded and comprises between about 10 and about 30 bases.

Embodiment 25. The composition of embodiment 18, wherein the polynucleotide is double-stranded and comprises between about 5 and about 2500 base pairs.

Embodiment 26. The composition of embodiment 18, wherein the polynucleotide is double-stranded and comprises between about 5 and about 500 base pairs.

Embodiment 27. The composition of embodiment 18, wherein the polynucleotide is double-stranded and comprises between about 5 and about 15 base pairs.

Embodiment 28. The composition of embodiment 18, wherein the polynucleotide comprises an antisense oligonucleotide, mipomersen, patisiran, exondys, an siRNA, or an inflammatory bowel disease (IBD) targeting siRNA.

Embodiment 29. The composition of any one of embodiments 2-11, wherein the active pharmaceutical ingredient is a small molecule having a molecular weight of 1 kD or less.

Embodiment 30. The composition of any one of embodiments 1-29, wherein the polymer precursor comprises one or both of a monomer and an oligomer precursor to a polymer.

Embodiment 31. The composition of any one of embodiments 1-30, wherein the polymer precursor is selected from Table 1 or Table 2, or combinations thereof.

Embodiment 32. The composition of embodiment 31, wherein the monomer is dopamine, levodopa, norepinephrine, methyldopa, levodopa methyl ester, levodopa ethyl ester, or combinations thereof.

Embodiment 33. The composition of any one of embodiments 1-32, wherein the oxygen source is a substrate for an endogenous catalyst.

Embodiment 34. The composition of embodiment 33, wherein the oxygen source is urea hydrogen peroxide or hydrogen peroxide.

Embodiment 35. The composition of any one of embodiments 1-34, wherein the composition is in an oral dosage form.

Embodiment 36. The composition of embodiment 35, wherein the oral dosage form is a solution, a gel, a tablet, a powder, or a capsule.

Embodiment 37. The composition of embodiment 35, wherein the oral dosage form comprises one or more solutions, gels, tablets, powders, or capsules.

Embodiment 38. The composition of embodiment 35, wherein the oral dosage form is an enteric dosage form.

Embodiment 39. The composition of any one of embodiments 1-38, wherein the permeation enhancer is a carnitine.

Embodiment 40. The composition of embodiment 41, wherein the carnitine is an acyl carnitine.

Embodiment 41. The composition of embodiment 39 or 40, wherein the carnitine is selected from the group consisting of lauroylcarnitine, palmitoylcarnitine, and palmitoyl carnitine chloride (PCC).

Embodiment 42. The composition of embodiment 39 or 40, wherein the carnitine is lauroylcarnitine or palmitoylcarnitine.

Embodiment 43. The composition of any one of embodiments 1-38, wherein the permeation enhancer is a choline.

Embodiment 44. The composition of embodiment 43, wherein the choline is lysophosphatidyl choline.

Embodiment 45. The composition of any one of embodiments 1-38, wherein the permeation enhancer is an aromatic alcohol.

Embodiment 46. The composition of embodiment 45, wherein the aromatic alcohol is selected from the group consisting of propyl gallate, butylated hydroxytoluene, and butylated hydroxyanisole.

Embodiment 47. The composition of embodiment 46, wherein the aromatic alcohol is benzyl alcohol, phenyl alcohol, or phenoxyethanol

Embodiment 48. The composition of any one of embodiments 1-38, wherein the permeation enhancer is a piperazine derivative.

Embodiment 49. The composition of embodiment 48, wherein the piperazine derivative is selected from the group consisting of 1-phenylpiperazine, 1-methyl-4-phenylpiperazine, 1-(4-methylphenyl)piperazine, and 1-benzylpiperazine.

Embodiment 50. The composition of embodiment 49, wherein the piperazine derivative is 1-phenylpiperazine or 1-methyl-4-piperazine.

Embodiment 51. The composition of any one of embodiments 1-38, wherein the permeation enhancer is a mucoadhesive polymer.

Embodiment 52. The composition of embodiment 51, wherein the mucoadhesive polymer is selected from the group consisting of chitosan, chitosan hydrochloride, trimethylated chitosan chloride, and N,N,N-trimethyl chitosan chloride.

Embodiment 53. The composition of embodiment 52, wherein the mucoadhesive polymer is trimethylated chitosan chloride.

Embodiment 54. The composition of any one of embodiments 1-38, wherein the permeation enhancer is a cell penetrating peptide.

Embodiment 55. The composition of embodiment 54, wherein the cell penetrating peptide is selected from the group consisting of transportan and penetratin.

Embodiment 56. The composition of embodiment 54, wherein the cell penetrating peptide is selected from the group consisting of oligoarginines, polyarginines, oligolysines, polylysines, oligotryptophans, and polytryptophan.

Embodiment 57. The composition of any one of embodiments 1-38, wherein the permeation enhancer is an amino acid.

Embodiment 58. The composition of embodiment 57, wherein the amino acid is tryptophan.

Embodiment 59. The composition of any one of embodiments 1-38, wherein the permeation enhancer is an ionic liquid.

Embodiment 60. The composition of embodiment 59, wherein the ionic liquid is selected from the group consisting of choline geranate, nicotinic acid and trigonelline.

Embodiment 61. The composition of embodiment 59 or 60, wherein the ionic liquid is choline geranate.

Embodiment 62. The composition of any one of embodiments 1-38, wherein the permeation enhancer is an organic solvent.

Embodiment 63. The composition of embodiment 62, wherein the solvent is selected from the group consisting of ethanol, 2-propanol, 1-propanol, and 2-methyl-2-propanol.

Embodiment 64. The composition of embodiment 62 or 63, wherein the organic solvent is selected from the group consisting of dimethyl sulfoxide, ethyl acetate, and acetone.

Embodiment 65. The composition of any one of embodiments 1-38, wherein the permeation enhancer is an anionic surfactant.

Embodiment 66. The composition of embodiment 65, wherein the anionic surfactant is sodium dodecyl sulphate, or an alternative pharmaceutically acceptable salt thereof.

Embodiment 67. The composition of embodiment 65, wherein the anionic surfactant is sodium cholate, or an alternative pharmaceutically acceptable salt thereof.

Embodiment 68. The composition of any one of embodiments 1-38, wherein the permeation enhancer is a chelating agent.

Embodiment 69. The composition of embodiment 68, wherein the chelating agent is selected from the group consisting of EDTA, EGTA, and DTPA.

Embodiment 70. The composition of embodiment 68 or 69, wherein the chelating agent is EDTA.

Embodiment 71. The composition of any one of embodiments 1-38, wherein the permeation enhancer is a non-ionic surfactant.

Embodiment 72. The composition of embodiment 71, wherein the non-ionic surfactant is an ethoxylate.

Embodiment 73. The composition of embodiment 71, wherein the non-ionic surfactant is an alcohol ethoxylate (C_{X}E_{Y}, where X is the alcohol carbon number and Y is the ethylene oxide number).

Embodiment 74. The composition of embodiment 71, wherein the non-ionic surfactant is a medium or long chain fatty acid sugar ester.

Embodiment 75. The composition of embodiment 71, wherein the non-ionic surfactant is a medium or long chain fatty acid sucrose ester.

Embodiment 76. The composition of embodiment 71, wherein the non-ionic surfactant is an ethoxylated fatty acid sugar ester.

Embodiment 77. The composition of embodiment 71, wherein the non-ionic surfactant is an ethoxylated sorbitan ester.

Embodiment 78. The composition of embodiment 71, wherein the non-ionic surfactant is an ethoxylated glyceride.

Embodiment 79. The composition of embodiment 71, wherein the non-ionic surfactant is selected from the group consisting of macrogol-8 glyceride, sucrose esters, sucrose laurate, ethoxylates, alkyl maltosides, dodecyl maltoside, short chain polyethylene glycol, Brij® series, polyoxyethylene(10) oleyl ether, polyoxyethylene (23) lauryl ether, polysorbates, polysorbate series PS 20, PS 40, PS 60, PS 65, PS 80, and Triton X-100.

Embodiment 80. The composition of embodiment 71, wherein the non-ionic surfactant is caprylocaproyl polyoxyl-8 glyceride (LABRASOL®), poloxamers, polyoxylglycerides, or polyethylene monostearate.

Embodiment 81. The composition of any one of embodiments 1-38, wherein the permeation enhancer is a non-ionic detergent.

Embodiment 82. The composition of embodiment 81, wherein the non-ionic detergent is sucrose monolaurate or n-tetradecyl ß-D-maltopyranoside (TDM).

Embodiment 83. The composition of embodiment 81, wherein the non-ionic detergent is sucrose monolaurate.

Embodiment 84. The composition of any one of embodiments 1-38, wherein the non-ionic detergent is n-tetradecyl ß-D-maltopyranoside (TDM).

Embodiment 85. The composition of any one of embodiments 1-38, wherein the permeation enhancer is a fatty acid, a fatty acid salt, an ethoxylated fatty acid ester, a sugar fatty acid ester, or an ethoxylated sugar fatty acid ester.

Embodiment 86. The composition of embodiment 85, wherein the fatty acid salt is sodium caprate (C₁₀), or an alternative pharmaceutically acceptable salt thereof.

Embodiment 87. The composition of embodiment 85, wherein the fatty acid salt is sodium caprylate (C₈), or an alternative pharmaceutically acceptable salt thereof, or sodium laurate (C₁₂), or alternative pharmaceutically acceptable salt thereof.

Embodiment 88. The composition of embodiment 85, wherein the sugar fatty acid ester is a fatty acid ester of a monosaccharide.

Embodiment 89. The composition of embodiment 85, wherein the sugar fatty acid ester is an ethoxylated fatty acid ester of a monosaccharide.

Embodiment 90. The composition of embodiment 85, wherein the sugar fatty acid ester is a fatty acid ester of sorbitan or glucose.

Embodiment 91. The composition of embodiment 85, wherein the sugar fatty acid ester is an ethoxylated fatty acid ester of sorbitan or glucose.

Embodiment 92. The composition of embodiment 85, wherein the sugar fatty acid ester is a fatty acid ester of a disaccharide.

Embodiment 93. The composition of embodiment 85, wherein the sugar fatty acid ester is an ethoxylated fatty acid ester of a disaccharide.

Embodiment 94. The composition of embodiment 85, wherein the sugar fatty acid ester is a fatty acid ester of sucrose or maltose.

Embodiment 95. The composition of embodiment 85, wherein the sugar fatty acid ester is an ethoxylated fatty acid ester of sucrose or maltose.

Embodiment 96. The composition of embodiment 85, wherein the fatty acid, ethoxylated fatty acid ester, sugar fatty acid ester, or ethoxylated sugar fatty acid ester is selected from the group consisting of dodecylmaltoside, sodium dodecyl sulfate, nonaethylene glycol monododecyl ether (C₁₂E₉), sodium laurate (C₁₂), sodium nonanoate (C₉), sodium undecanoate (C₁₁), sodium undecylenate (C11:1), sodium oleate, linoleic acid, and sucrose monolaurate, and pharmaceutically acceptable salts thereof or alternative pharmaceutically acceptable salts thereof.

Embodiment 97. The composition of embodiment 85, wherein the fatty acid, ethoxylated fatty acid ester, sugar fatty acid ester, or ethoxylated sugar fatty acid ester is nonaethylene glycol monododecyl ether (C₁₂E₉).

Embodiment 98. The composition of any one of embodiments 1-38, wherein the permeation enhancer is an endogenous secretion.

Embodiment 99. The composition of embodiment 98, wherein the endogenous secretion is a bile salt.

Embodiment 100. The composition of embodiment 99, wherein the bile salt is selected from the group consisting of sodium taurodeoxycholate, sodium taurocholate, sodium cholate, sodium deoxycholate, sodium glycodeoxycholate, sodium glycochenodeoxycholate, sodium glycocholiate, sodium chenodeoxycholate, sodium taurochenodeoxycholate, sodium lithocholate, and mixed sodium taurodihydrofusidate, or an alternative pharmaceutically acceptable salt thereof.

Embodiment 101. The composition of embodiment 98 or 99, wherein the bile salt is sodium cholate, or an alternative pharmaceutically acceptable salt thereof.

Embodiment 102. The composition of any one of embodiments 1-38, wherein the permeation enhancer is an N-acylated acid.

Embodiment 103. The composition of embodiment 102, wherein the N-acylated acid is acetylsalicylic acid, or a pharmaceutically acceptable salt thereof.

Embodiment 104. The composition of embodiment 102, wherein the N-acylated acid is sodium N-[8-(2-hydroxybenzoyl)amino]caprylate (SNAC), or an alternative pharmaceutically acceptable salt thereof, or the corresponding acid thereof.

Embodiment 105. The composition of embodiment 102, wherein the N-acylated acid is 8-(N-2-hydroxy-5-chloro-benzoyl)-amino-caprylic acid (5-CNAC), or an alternative pharmaceutically acceptable salt thereof, or the corresponding acid thereof.

Embodiment 106. The composition of embodiment 102, wherein the N-acylated acid is 4-[(4-chloro-2-hydroxy-benzoyl)amino]butanoic acid (4-CNAB), or an alternative pharmaceutically acceptable salt thereof, or the corresponding acid thereof.

Embodiment 107. The composition of embodiment 102, wherein the N-acylated acid is N-(10-[2-hydroxybenzoyl]-amino)decanoic acid (SNAD), or an alternative pharmaceutically acceptable salt thereof, or the corresponding acid thereof.

Embodiment 108. The composition of embodiment 102, wherein the N-acylated acid is monosodium N-(4-chlorosalicyloyl)-4-aminobutyrate (5-CNAB), or an alternative pharmaceutically acceptable salt thereof, or the corresponding acid thereof.

Embodiment 109. The composition of embodiment 102, wherein the N-acylated acid is N-[8-(2-hydroxy-4-methoxy)benzoyl]amino caprylic acid (4-MOAC), or a pharmaceutically acceptable salt thereof.

Embodiment 110. The composition of embodiment 102, wherein the N-acylated acid is sodium N-[8-(2-hydroxybenzoyl)amino]caprylate (SNAC), or an alternative pharmaceutically acceptable salt thereof, or the corresponding acid thereof.

Embodiment 111. The composition of any one of embodiments 1-38, wherein the permeation enhancer is a high molecular weight polymer.

Embodiment 112. The composition of embodiment 111, wherein the high molecular weight polymer is a polysaccharide.

Embodiment 113. The composition of embodiment 111, wherein the high molecular weight polymer is an antibacterial toxin.

Embodiment 114. The composition of embodiment 113, wherein the antibacterial toxin is selected from the group consisting of zonula occludens toxin analogs, viral protein 8 analogs, and Clostridium perfringens enterotoxin analogs.

Embodiment 115. The composition of embodiment 111, wherein the high molecular weight polymer is chitosan or carboxymethylcellulose.

Embodiment 116. The composition of any one of embodiments 1-38, wherein the permeation enhancer is a caprylocaproyl PEG 8 glyceride.

Embodiment 117. The composition of any one of embodiments 1-38, wherein the permeation enhancer is a sugar-based surfactant.

Embodiment 118. The composition of embodiment 117, wherein the sugar-based surfactant is dodecyl-ß-D-maltopyranoside (DDM).

Embodiment 119. The composition of any one of embodiments 1-38, wherein the permeation enhancer is glyceryl monocaprate.

Embodiment 120. The composition of any one of embodiments 1-38, wherein the permeation enhancer is urea.

Embodiment 121. The composition of any one of embodiments 1-38, wherein the permeation enhancer is sodium docusate, or an alternative pharmaceutically acceptable salt thereof, or the corresponding acid thereof.

Embodiment 122. The composition of any one of embodiments 1-38, wherein the permeation enhancer is citric acid, or a pharmaceutically acceptable salt thereof.

Embodiment 123. The composition of any one of embodiments 1-122, wherein the permeation enhancer enhances paracellular transport.

Embodiment 124. The composition of any one of embodiments 1-122, wherein the permeation enhancer enhances transcellular transport.

Embodiment 125. The composition of any one of embodiments 1-122, wherein the permeation enhancer enhances paracellular transport and transcellular transport.

Embodiment 126. A method of forming a polymer coating in the small intestine of a subject, the method comprising administering to a subject a composition of any one of embodiments 1-125.

Embodiment 127. A method of forming a polymer coating in the small intestine of
a subject, the method comprising orally administering to a subject:
a polymer precursor;
an oxygen source; and
a permeation enhancer that enhances uptake of one or more active pharmaceutical ingredients;
wherein the polymer precursor and the oxygen source contact a catalyst endogenous to the subject and the catalyst polymerizes the polymer precursor.

Embodiment 128. The method of embodiment 127, further comprising administering an active pharmaceutical ingredient to the subject.

Embodiment 129. The method of embodiment 127, wherein the polymer precursor, the oxygen source and the permeation enhancer are administered as a single composition.

Embodiment 130. The method of embodiment 128, wherein the polymer precursor, the oxygen source, the permeation enhancer, and the active pharmaceutical ingredient are administered as a single composition.

Embodiment 131. The composition of embodiment 14, wherein the polypeptide comprises semaglutide.

Embodiment 132. The composition of embodiment 14, wherein the polypeptide comprises tirzepatide.

### EXAMPLES

The disclosure is further illustrated by the following non-limiting examples.

### Example 1

### Initial Evaluation of Permeation Enhancers—by 96 Well Plate Screening

Methods: Fresh porcine jejunum tissue was resected and washed in a series of saline solutions supplemented with 5% antibiotic-antimycotic solution. The tissue was then mounted onto an augmented 96-well plate (the plate that will receive permeated drug) filled with buffer. Tissues from multiple pigs were used in order to be able to calculate an average permeability and standard deviations from the mean. Another 96-well plate was sandwiched on top and secured by magnets. To prepare the donor solutions, a master plate was generated by aliquoting from 3 plates: (1) The excipient and API plate included the excipients and drug at 2.5× the final concentration. The tris buffer and hydrogen peroxide were at 1.25× the final concentration. (2) The polydopamine (PDA) plate had PDA at 2.5× the final concentration, and tris buffer and hydrogen peroxide at 1.25× the final concentration. (3) The dopamine plate had dopamine at 5× the final concentration. All formulations contained GSEL_40 (9.8 mg/mL dopamine HCl, 25 mg/ml PDA, 0.67 mg/mL hydrogen peroxide, 2.2 mg/mL Tris HCl, 4.3 mg/mL Tris base. The master plate was aliquoted from each plate at 0.5:0.5:1 volume ratio. Then the donor solution in the master plate was pipetted into the donor 96-well plate. The entire system was then covered and placed at room temperature for 20-24 hours. Samples from the receiving 96-well plate were then analyzed using HPLC. Experimental samples and control samples tested on the same tissue were compared to determine percent mean permeability and/or fold change. Permeability was calculated by comparing permeate to how much was added to the donor chamber. Fold change was calculated by comparing experimental samples to control samples. Excipients tested and their corresponding names are listed in Table E1.

TABLE E1
Excipient table

| Excipient | Name |
|---|---|
| C08 | Sodium Octanoate |
| C10 | Decanoic Acid |
| CALC | Calcium Chloride |
| CCO:75n | Calcium Carbonate Nanoparticles, |
| | Cubic, 75-105 nm |
| CHA | Cholic Acid Sodium Salt |
| CHAPS | CHAPS hydrate |
| CHLORG | Chlorogenic acid |
| CINA | Cinnamic Acid Sodium Salt |
| CITR | Sodium Citrate |
| DOC | Docusate Sodium |
| EDTA | Ethylenediaminetetraacetic Acid |
| | Tetrasodium Salt |
| EPE:2 | Poly(ethylene glycol)-block- |
| | Poly(propylene glycol)-block- |
| | Poly(ethylene glycol) 2.8K |
| GAPS | Gibberellic Acid Potassium Salt |
| GCA | Sodium Glycocholate |
| GCDCA | Glycochenodeoxyxholic Acid |
| | Sodium Salt |
| GERAN | Geranic Acid |
| HAP:5u | Hydroxyapatite, Spherical, 5 um |
| HCO3 | Sodium bicarbonate |
| HPBCD | (2-Hydroxypropyl)-beta cyclodextrin |
| HPGCD | (2-Hydroxypropyl)-gamma-cyclodextrin |
| | 4.5 DS |
| IAA | Indole 3-acetic acid Sodium Salt |
| KCL | Potassium Chloride |
| LABS | Caprylcaproyl Macrogol Glycerides |
| | (Labrasol) |
| LFCS | Oleoyl polyoxyl-6 glycerides |
| NHCO | Ammonium Carbonate |
| OX | Bile (BOVINE) (Sigma Aldrich |
| | Catalog #b3883) |
| PEG:1 | Poly(ethylene glycol) 1K |
| PEG:6 | Poly(ethylene glycol) 6K |
| PEI:B25 | Poly(ethylenimine) Hydrochloride, |
| | Branched 25 KDa |
| PEI:L4 | Poly(ethylenimine) Hydrochloride, |
| | Linear 4k |
| PEP:2 | Poly(propylene glyco)-block- |
| | poly(ethylene glycol)-block- |
| | poly(propylene glycol) Mn ~2000 |
| PHYT | Phytic Acid Sodium Salt Hydrate |
| POXZ:5 | Poly(2-ethyl-2-oxazoline) 5K |
| PVA:30 | Poly(vinyl alcohol) 30-70k |
| PVSA | Polyvinyl Sulfonic Acid |
| SBECD | Sulfobutylated beta-cyclodextrin |
| | sodium salt |
| SDS | Sodium dodecyl sulfate |
| SHB | Sodium 4-hydroxybenzoate |
| SNAC | Salcaprozate Sodium |
| SPMD | Spermidine Trihydrochloride |
| VB9 | Folic Acid |
| ZnO:R10n | Zinc Oxide Nanoparticles Round 10 nm |
| AKETG | alpha-Ketoglutaric acid disodium salt |
| AVICEL | Microcrystalline cellulose |
| C12E9 | Nonaethylene glycol monododecyl ether |
| C12 | Dodecanoic acid |
| C14 | Tetradecanoic acid |
| CAP90 | Propylene glycol monocaprylate |
| CCO:75N | Calcium carbonate nanoparticles, cubic |
| | 75-105 nm |
| CDCA | Chenodeoxycholic acid sodium salt |
| CEACD | (2-Carboxyethyl)-alpha-cyclodextrin |
| | 3 DS |
| CLD | Calcium levulinate dihydrate |
| DADMAC | Diallyldimethylammonium chloride |
| | solution |
| DARAB | D-(−)-Arabinose |
| DCA | Deoxycholic acid sodium salt |
| DENDAC2.5 | PAMAM dendrimer carboxylate |
| | surface groups G2.5 |
| DENDOH4 | PAMAM dendrimer hydroxyl surface |
| | groups G4 |
| DGAL | D-(+)-Galactose |
| DGLC | D-(+)-Glucose |
| DGLC6P | D-glucose 6-phosphate sodium salt |
| DMANIT | D-Mannitol |
| DSORB | D-Sorbitol |
| DTOSE | D-Trehalose |
| EMUB25 | Eumulgin ® 25 |
| EPE:2 | Poly(ethylene glycol)-block- |
| | Poly(propylene glycol)-block- |
| | Poly(ethylene glycol) 2.8K |
| GAPS | Gibberellic acid potassium salt |
| GCA | Sodium Glycocholate |
| GCDCA | Glycochenodeoxycholic acid sodium salt |
| GDCA | Glycodeoxycholic acid sodium salt |
| GERAN | Geranic acid |
| GRRHZ | Glycyrrhizin |
| GSEL | Gastrointestinal synthetic epithelial lining |
| GUAN | Disodium guanylate |
| HAP:10U | Hydroxyapatite, spherical, 10 um |
| HAP:5U | Hydroxyapatite, spherical, 5 um |
| HAP: | Hydroxyapatite nanoparticles, |
| R40X170N | Rod-shaped, 40 × 170 nm |
| HDE | Hydroxyectoine |
| HPACD | (2-Hydroxypropyl)-alpha-cyclodextrin |
| | 4.5 DS |
| HPBCD | 2-Hydroxypropyl beta cyclodextrin |
| HPGCD | (2-hydroxypropyl)-gamma-cyclodextrin |
| | 4.5 DS |
| IAA | Indole 3-aetic acid sodium salt |
| IMP | Inosine 5′-monophosphate disodium |
| | salt hydrate |
| K188 | Poloxamer 188 |
| KCL | Potassium chloride |
| LABS | Caprylcaproyl macrogool glycerides |
| LCARN | L-carnitine hydrochloride |
| LFCS | Oleoyl polyoxyl-6 glycerides |
| MAIS | Linoleic acid monoglyceride |
| NAC | N-Acetyl-L-cysteine |
| NANB | Sodium 4-(nicotinamido)butanoate |
| NHBICA | Ammonium bicarbonate |
| NHCO | Ammonium carbonate |
| NHP | Ammonium phosphate dibasic |
| PEI:B25 | Poly(ethylnimine) branched 25K |
| PEP:2 | Poly(propylene glycol)-b- |
| | poly(ethylene glycol)-b-poly |
| | (propylene glycol) |
| PHYT | Phytic acaid sodium salt hydrate |
| PVP:10 | Poly(vinylpyrrolidone) 10K |
| PVP:40 | Poly(vinylpyrrolidone) 40K |
| PVSA | Polyvinyl sulfonic acid |
| QUILZ | Quillaja saponin |
| SBECD | Sulfobutylated beta-cyclodextrin |
| | sodium salt |
| SDS | Sodium dodecyl sulfate |
| SHB | Sodium 4-hydroxybenzoate |
| SNAC | Salcaprozate sozium |
| SPER | Spermidine tetrahydrochloride |
| SPMD | Spermidine trihydrochloride |
| STC | Sodium taurocholate |
| SUCGCD | Succinyl-gamma-cyclodextrin 3.5 DS |
| TDCA | Taurodeoxycholic acid sodium salt |
| UREA | Urea |
| XYLI | Xylitol |
| ZNO:R10N | Zinc oxide powder, rod-shaped, 10-30 nm |
| BC10 | Brij C10 |
| BC2 | SP Brij C2 |
| BO20 | Brij O20 |
| C12E9 | Nonaethylene glycol monododecyl ether |
| EMUB25 | Eumulgin ® 25 |
| GCDCA | Glycochenodeoxycholic acid sodium salt |
| Span: 20 | Span 20 |
| Tris_50 | 50 mM Tris |

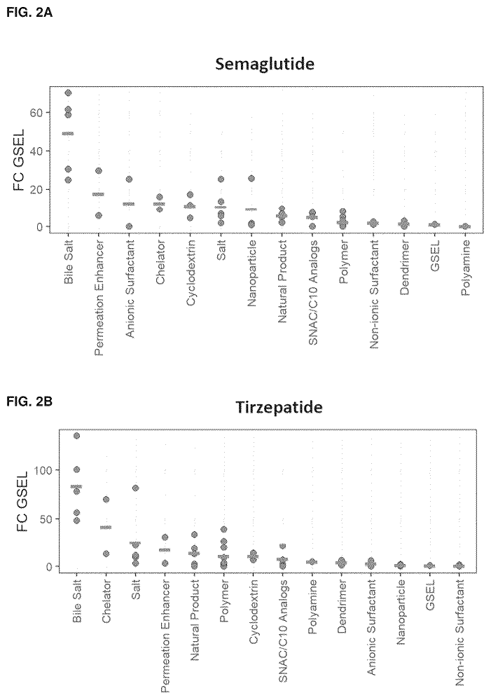

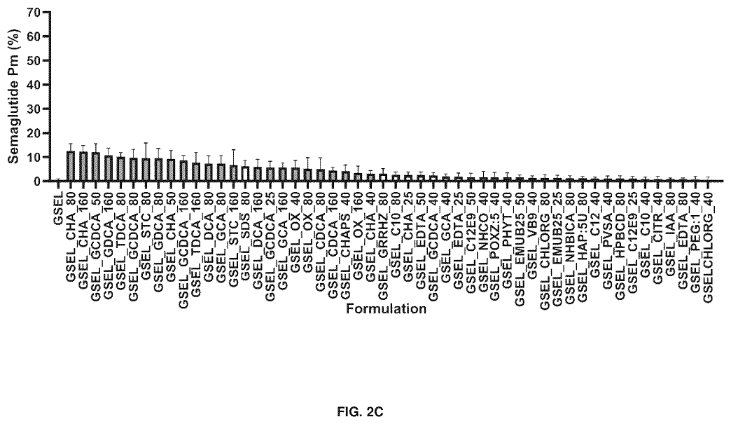

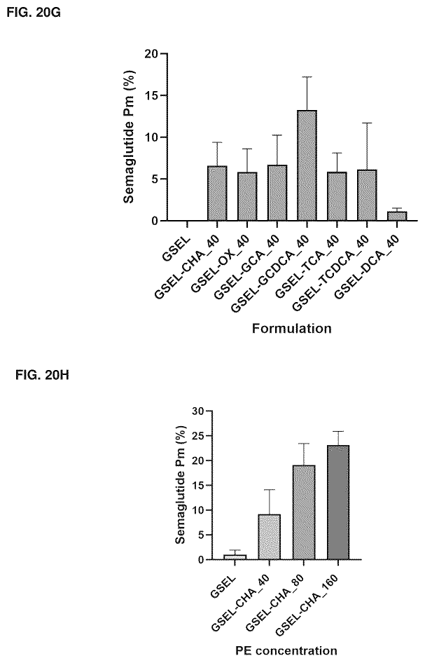

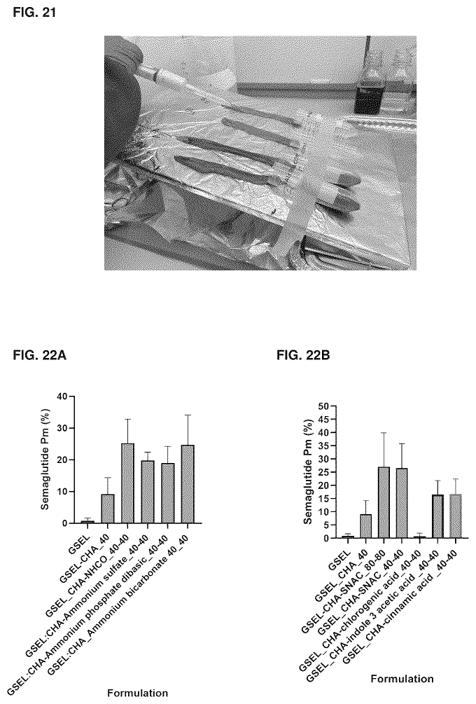

Results: Active pharmaceutical ingredient (API) permeation was significantly enhanced when the GSEL was combined with select single excipients (FIG. 1A, FIG. 1B, FIG. 1C, FIG. 1D, Table E1). Excipients belonging to specific classes such as bile salts and chelators were identified to significantly increase API permeability compared to GSEL alone (FIG. 2A, FIG. 2B, FIG. 2C). After 50 single excipients were screened with semaglutide, the bile salt family was the most effective class of excipients at enhancing permeability of semaglutide, with a 10% permeability when combined with GSEL (FIG. 2C). These results were corroborated by Franz cell tests on duodenum tissue, where increasing CHA concentrations also led to an increasing permeability (FIG. 21I).

The effect of excipient concentration on API permeability was also evaluated (FIG. 1F, 1G). Concentration influenced API permeability for some excipients, but did not impact mean permeability for others. Increasing excipient concentrations above 25 mg/mL resulted in diminishing improvements in permeability.

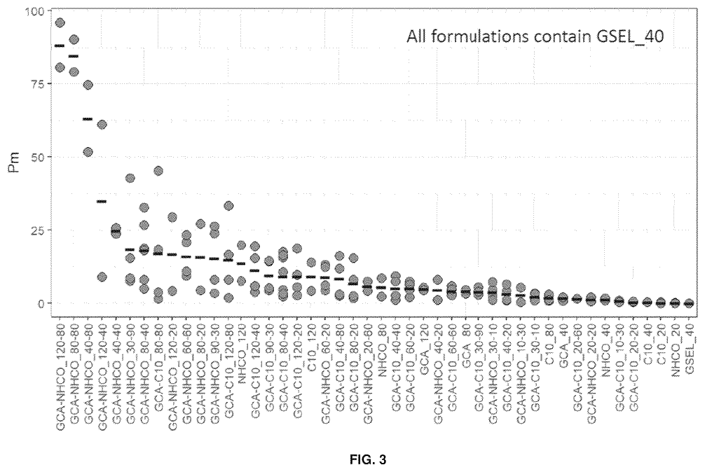

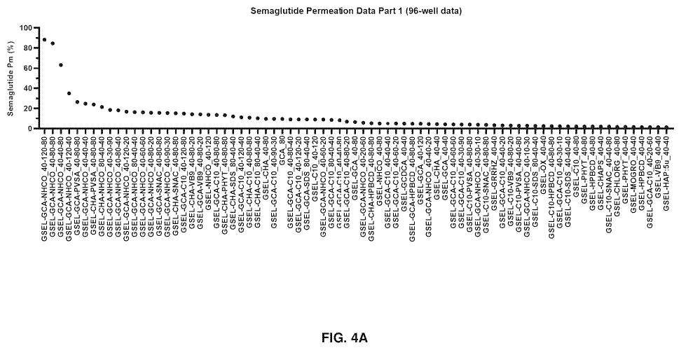

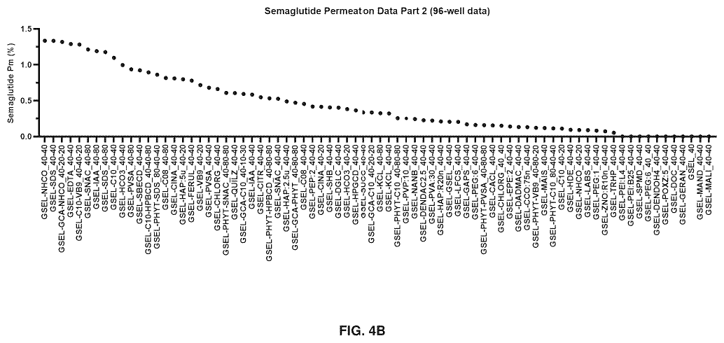

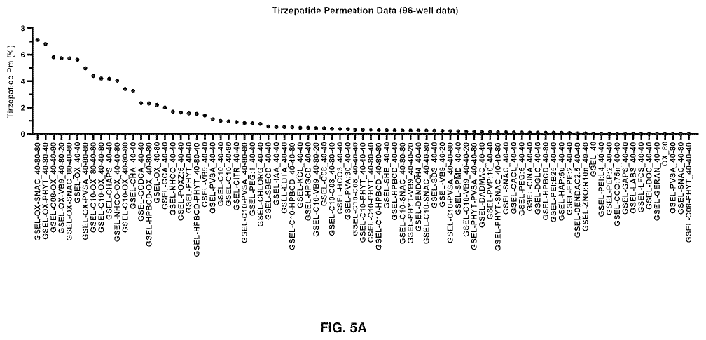

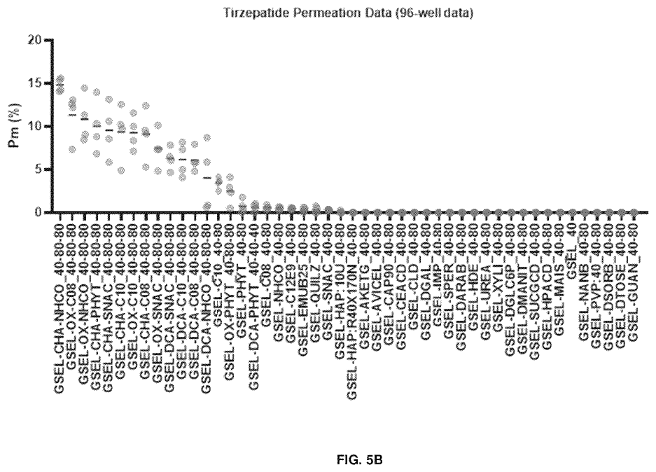

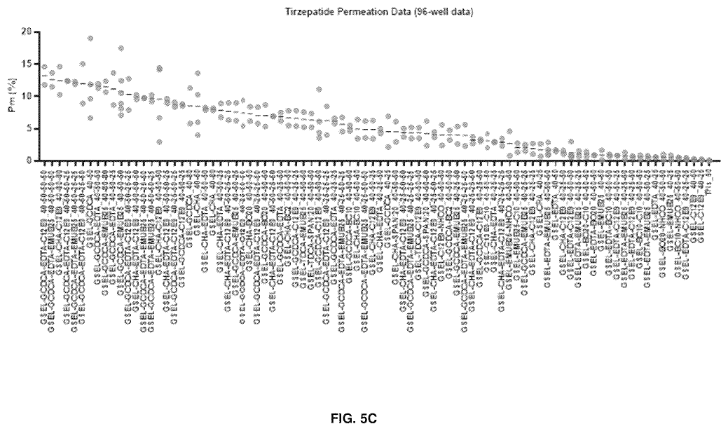

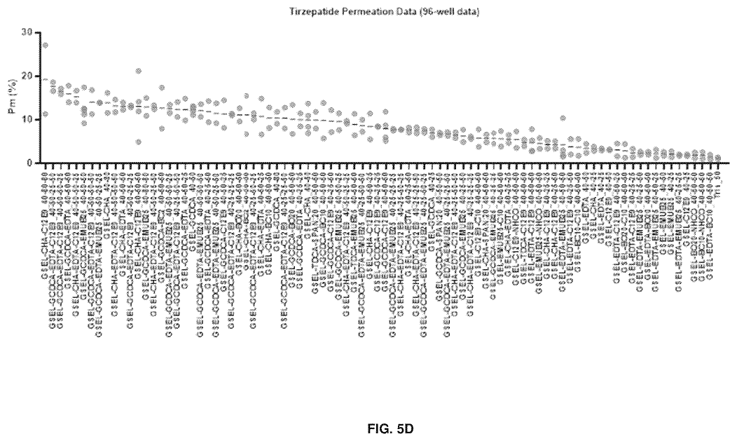

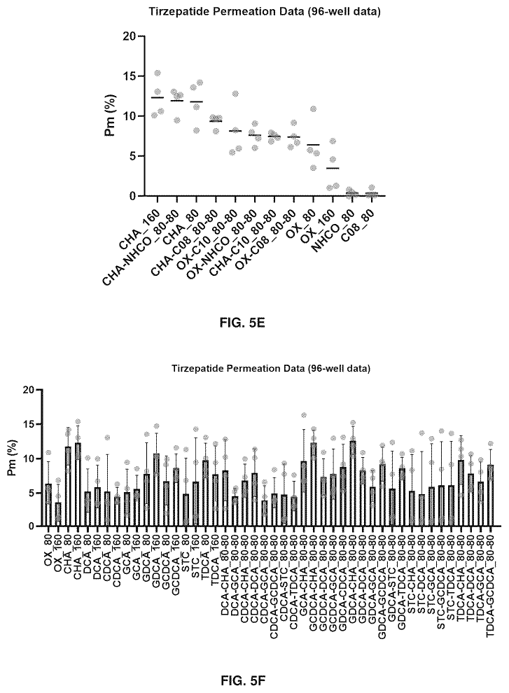

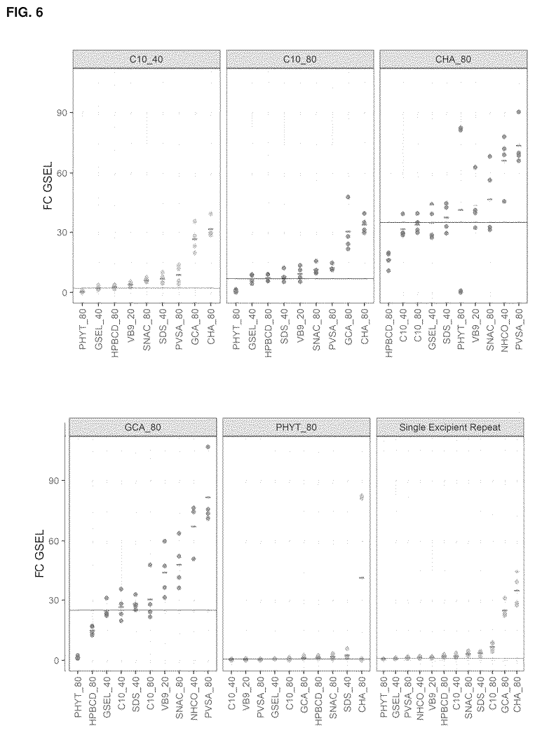

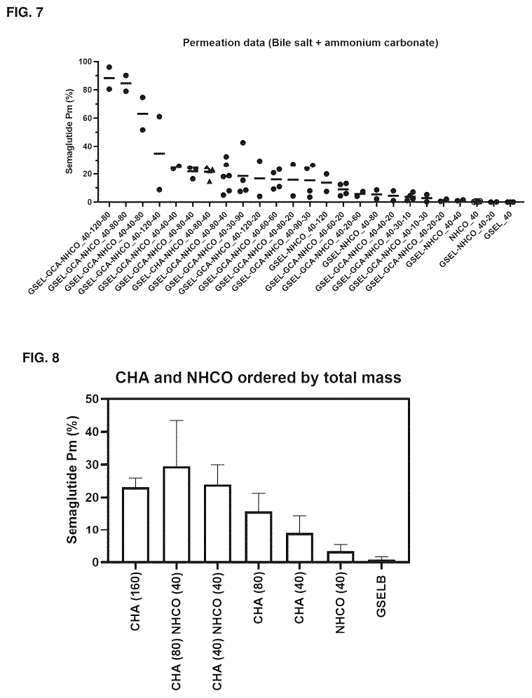

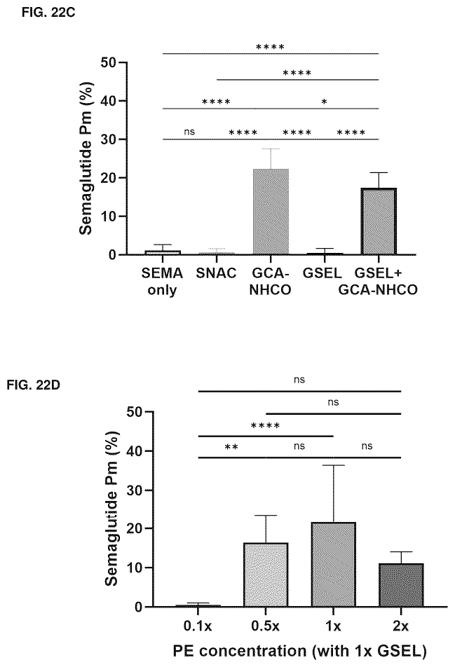

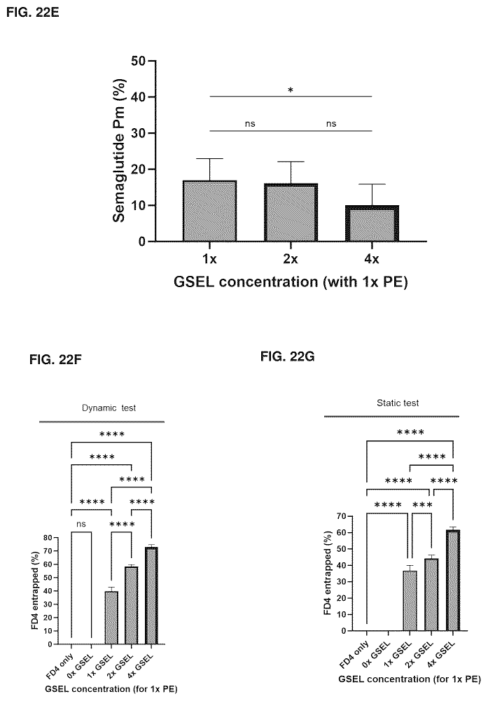

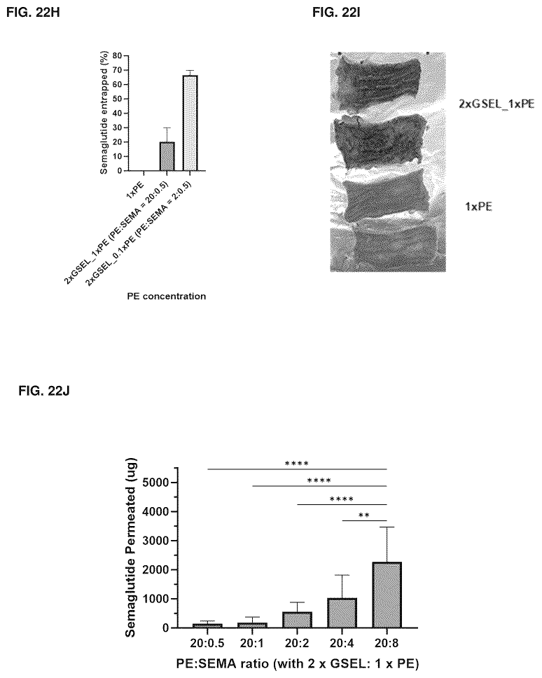

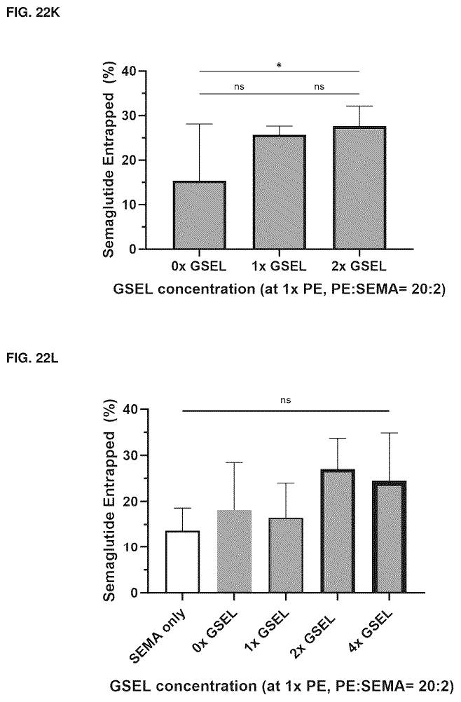

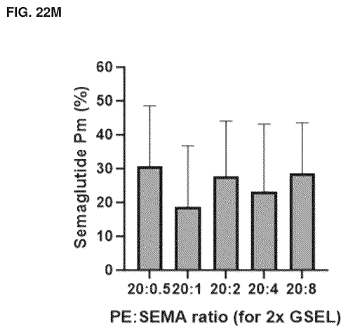

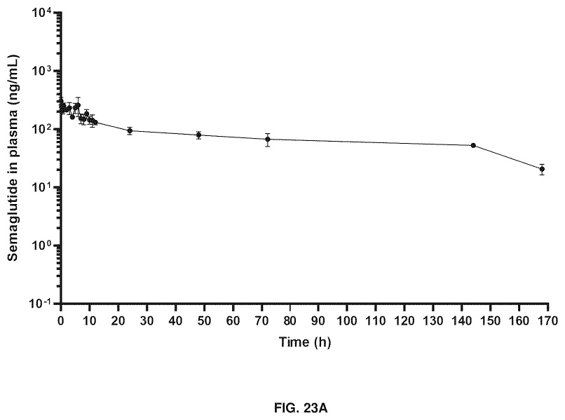

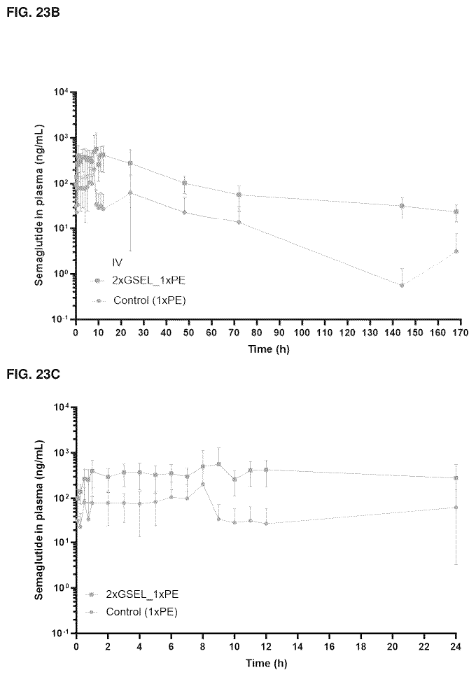

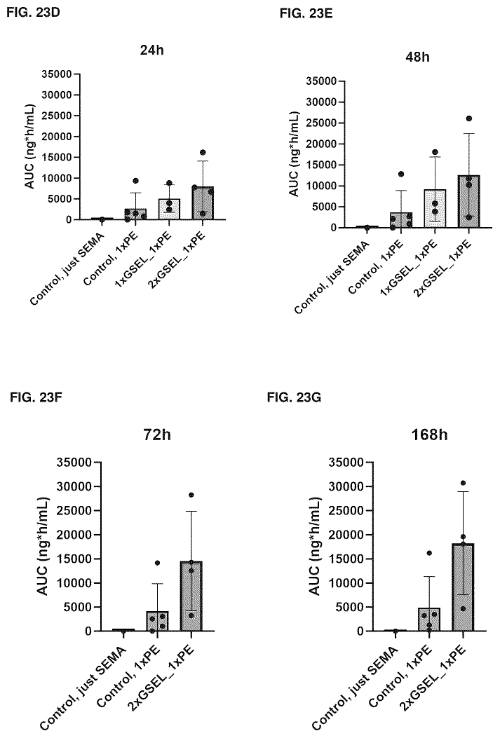

Excipients were also tested in combination with each other, and with varying ratios of formulations (FIG. 3, FIG. 4A, FIG. 4B, FIG. 5A, FIG. 5B, FIG. 5C, FIG. 5D, FIG. 5E, FIG. 5F, Table E2, Table E3, Table E4, Table E5). Top combinations of excipients that enhanced permeability included at least one bile salt, cholic acid or a salt thereof, or glycocholic acid or a salt thereof. Further permeability tests on Franz demonstrated the same synergistic effect when a bile salt is combined with other ammonium salts, (FIG. 22B). Based on the dual excipient screen, another combination that presented a synergistic effect was the combination of bile salts CHA and GCA with SNAC (FIG. 4A, FIG. 18A, FIG. 18B). This was also supported by supplementary tests on Franz cells where additional benzene ring-based molecules were tested (FIG. 23A) Bile salt combinations of note included 80 mg/mL poly(vinylsulfonic acid) sodium salt, 40 mg/mL ammonium carbonate, 80 mg/mL salcaprozate sodium, 20 mg/mL folic acid, 80 mg/mL phytic acid, and 40 mg/mL sodium dodecyl sulfate (FIG. 6, FIG. 7, FIG. 8, Table E6).

TABLE E2
Mean permeability of tirzepatide
from 96 well plate screen

| Formulation ID | Pm (%) |
|---|---|
| GSEL-CHA_40-80 | 14.617225 |
| GSEL-STC_40-80 | 14.3403775 |
| GSEL-GCDCA_40-80 | 12.6334625 |
| GSEL-GDCA_40-80 | 11.37472575 |
| GSEL-OX_40-80 | 11.2640775 |
| GSEL-TDCA_40-80 | 10.5118075 |
| GSEL-GCA_40-80 | 9.494401 |
| GSEL-DCA_40-80 | 9.4135005 |
| GSEL-SDS_40-80 | 6.18036225 |
| GSEL-CDCA_40-80 | 4.8189925 |
| GSEL-C10_40-80 | 3.363229 |
| GSEL-GRRHZ_40-80 | 3.152514 |
| GSEL-HPBCD_40-80 | 2.56573275 |
| GSEL-CHLORG_40-80 | 1.39729125 |
| GSEL-HAP:5U_40-80 | 1.308657 |
| GSEL-NHBICA_40-80 | 1.24080575 |
| GSEL-NHCO_40-80 | 1.03374675 |
| GSEL-SNAC_40-80 | 0.94380975 |
| GSEL-IAA_40-80 | 0.87040475 |
| GSEL-PVSA_40-80 | 0.75541025 |
| GSEL-SBECD_40-80 | 0.73141475 |
| GSEL-PEG:1_40-80 | 0.68227725 |
| GSEL-ZNO:R10N_40-80 | 0.5287995 |
| GSEL-C08_40-80 | 0.5231195 |
| GSEL-PVP:10_40-80 | 0.45121925 |
| GSEL-K188_40-80 | 0.40641325 |
| GSEL-PEP:2_40-80 | 0.39455775 |
| GSEL-SHB_40-80 | 0.38235425 |
| GSEL-GAPS_40-80 | 0.38149425 |
| GSEL-LFCS_40-80 | 0.3741175 |
| GSEL-DADMAC_40-80 | 0.370069 |
| GSEL-HPGCD_40-80 | 0.35656225 |
| GSEL-CCO:75N_40-80 | 0.32975825 |
| GSEL-GERAN_40-80 | 0.31747375 |
| GSEL-DENDAC2.5_40-80 | 0.3140605 |
| GSEL-NHP_40-80 | 0.26399475 |
| GSEL-DGLC_40-80 | 0.25976275 |
| GSEL-DENDOH4_40-80 | 0.25540425 |
| GSEL-PHYT_40-80 | 0.23173875 |
| GSEL_40 | 0.229966 |
| GSEL-PEG:6_40-80 | 0.227398 |
| GSEL-PEI:B25_40-80 | 0.161895 |
| GSEL-KCL_40-80 | 0.158575 |
| GSEL-LABS_40-80 | 0.075777333 |
| GSEL-NAC_40-80 | 0.0685045 |
| GSEL-EPE:2_40-80 | 0.041631 |
| GSEL-SPMD_40-80 | 0 |
| GSEL-LCARN_40-80 | 0 |

TABLE E3
Mean permeability of semaglutide
from 96 well plate screen

| Formulation ID | Pm |
|---|---|
| GSEL-GCA-NHCO_40-120-80 | 88.28 |
| GSEL-GCA-NHCO_40-80-80 | 84.60 |
| GSEL-GCA-NHCO_40-40-80 | 63.10 |
| GSEL-GCA-NHCO_40-120-40 | 34.94 |
| GSEL-GCA-PVSA_40-80-80 | 26.43 |
| GSEL-GCA-NHCO_40-40-40 | 24.82 |
| GSEL-CHA-PVSA_40-80-80 | 23.81 |
| GSEL-CHA-NHCO_80-40-40 | 21.41 |
| GSEL-GCA-NHCO_40-30-90 | 18.49 |
| GSEL-GCA-NHCO_40-80-40 | 18.18 |
| GSEL-GCA-NHCO_40-120-20 | 16.77 |
| GSEL-GCA-NHCO_80-40-40 | 16.29 |
| GSEL-GCA-NHCO_40-60-60 | 16.08 |
| GSEL-GCA-NHCO_40-80-20 | 15.75 |
| GSEL-GCA-SNAC_40-80-80 | 15.59 |
| GSEL-GCS-NHCO_40-90-30 | 15.41 |
| GSEL-CHA-SNAC_40-80-80 | 15.20 |
| GSEL-GCA-C10_40-120-80 | 14.93 |
| GSEL-CHA-VB0_40-80-20 | 14.15 |
| GSEL-GCA-VB9_40-80-20 | 14.10 |
| GSEL-NHCO_40-120 | 13.71 |
| GSEL-GCA-C10_40-80-80 | 13.50 |
| GSEL-CHA-PHYT_40-80-80 | 13.29 |
| GSEL-CHA-SDS_80-40-40 | 12.05 |
| GSEL-GCA-C10_40-120-40 | 11.15 |
| GSEL-CHA-C10_40-80-80 | 10.88 |
| GSEL-CHA-C10_80-40-40 | 10.19 |
| GSEL-CHA_40-80 | 9.64 |
| GSEL-GCA-C10_40-90-30 | 9.57 |
| GCA_80 | 9.56 |
| GSEL-GCA-C10_40-80-40 | 9.15 |
| GSEL-GCA-C10_40-120-20 | 9.12 |
| GSEL-GCA-SDS_80-40-40 | 9.07 |
| GSEL-C10_40-120 | 9.05 |
| GSEL-GCA-NHCO_40-60-20 | 9.01 |
| GSEL-GCA-C10_80-40-40 | 8.56 |
| GSEL-GCA-C10_40-40-80 | 8.33 |
| GSEL-GCA-C10_40-80-20 | 6.83 |
| GSEL-GCA_40-80 | 6.49 |
| GSEL-GCA-NHCO_40-20-60 | 5.78 |
| GSEL-CHA-HPBCD_40-80-80 | 5.29 |
| GSEL-NHCO_40-80 | 5.09 |
| GSEL-GCA-C10_40-40-40 | 5.07 |
| GSEL-GCA-C10_40-60-20 | 5.02 |
| GSEL-GCDCA_40-40 | 4.95 |
| GSEL-GCA-HPBCD_40-80-80 | 4.84 |
| GSEL-GCA_40-120 | 4.84 |
| GSEL-GCA-NHCO_40-40-20 | 4.49 |
| GSEL-CHA_40-40 | 4.44 |
| GSEL-GCA_40-40 | 4.23 |
| GSEL-GCA-C10_40-60-60 | 4.08 |
| GSEL-GCA-C10_40-30-90 | 4.02 |
| GSEL-C10-PVSA_40-80-80 | 3.97 |
| GSEL-GCA-NHCO_40-30-10 | 3.79 |
| GSEL-C10-SNAC_40-80-80 | 3.71 |
| GSEL-GRRHZ_40-40 | 3.29 |
| GSEL-GCA-C10_40-40-20 | 3.11 |
| GSEL-C10-VB9_40-80-20 | 3.01 |
| GSEL-C10-PVSA_40-40-80 | 2.84 |
| GSEL-GCA-NHCO_40-10-30 | 2.78 |
| GSEL-C10-SDS_80-40-40 | 2.58 |
| GSEL-OX_40-40 | 2.45 |
| GSEL-C10-HPBCD_40-80-80 | 2.36 |
| GSEL-GCA-C10_40-30-10 | 2.26 |
| GSEL-C10-SDS_40-40-40 | 2.20 |
| GSEL-C10-40-80 | 2.12 |
| GSEL-PHYT_40-80 | 2.11 |
| GSEL-HPBCD_40-80 | 2.09 |
| GSEL-CHAPS_40-40 | 2.00 |
| GSEL-C10-SNAC_40-40-80 | 1.96 |
| GSEL-CHLORG_40-80 | 1.67 |
| GSEL-PHYT_40-40 | 1.65 |
| GSEL-MOGRO_40-40 | 1.53 |
| GSEL-HPBCD_40-40 | 1.49 |
| GSEL-GCA-C10_40-20-60 | 1.46 |
| GSEL-VBP_40-40 | 1.34 |
| GSEL-HAP:5u_40-40 | 1.34 |
| GSEL-NHCO_40-40 | 1.34 |
| GSEL-SDS_40-40 | 1.33 |
| GSEL-GCA-NHCO_40-20-20 | 1.32 |
| GSEL-EDTA_40-40 | 1.29 |
| GSEL-C10-VB0_40-40-20 | 1.28 |
| GSEL-SNAC_40-80 | 1.21 |
| GSEL-IAA_40-80 | 1.19 |
| GSEL-SDS_40-80 | 1.18 |
| GSEL-C10_40-40 | 1.10 |
| GSEL-HCO3_40-40 | 1.00 |
| GSEL-PVSA_40-80 | 0.94 |
| GSEL-SBECD_40-40 | 0.92 |
| GSEL-C10-HPBCD_40-40-80 | 0.89 |
| GSEL-PHYT-SDS_80-40-40 | 0.86 |
| GSEL-C08_40-40 | 0.82 |
| GSEL-CINA_40-40 | 0.81 |
| GSEL-HAP-5u_40-20 | 0.80 |
| GSEL-FERUL_40-40 | 0.78 |
| GSEL-VB9_40-20 | 0.72 |
| GSEL-PVSA_40-40 | 0.68 |
| GSEL-CHLORG_40-40 | 0.66 |
| GSEL-PHYT-SNAC_40-80-80 | 0.61 |
| GSEL-QUILZ_40-40 | 0.61 |
| GSEL-GCA-C10_40-10-30 | 0.59 |
| GSEL-IAA_40-40 | 0.58 |
| GSEL-CITR_40-40 | 0.55 |
| GSEL-PHYT-HPBCD_40-80-80 | 0.53 |
| GSEL-SNAC_40-40 | 0.53 |
| GSEL-HAP:2.5u_40-40 | 0.49 |
| GSEL-GCA-PHYT_40-80-80 | 0.47 |
| GSEL-C08_40-40 | 0.45 |
| GSEL-PEP:2_40-40 | 0.42 |
| GSEL-CINA_40-20 | 0.41 |
| GSEL-SHB_40-40 | 0.40 |
| GSEL-DGLC_40-40 | 0.40 |
| GSEL-HCO3_40-20 | 0.38 |
| GSEL-HPGCD_40-40 | 0.36 |
| GSEL-SUCC_40-40 | 0.34 |
| GSEL-GCA-C10_40-20-20 | 0.34 |
| GSEL-KCL_40-80 | 0.32 |
| GSEL-KCL_40-40 | 0.32 |
| GSEL-PHYT-C10_40-80-80 | 0.26 |
| GSEL-PVP:10_40-40 | 0.25 |
| GSEL-NANB_40-40 | 0.24 |
| GSEL-DENDAC2.5_40-40 | 0.23 |
| GSEL-PVA:30_40-40 | 0.22 |
| GSEL-HAP:R20n_40-40 | 0.21 |
| GSEL-GSEL_40-40 | 0.20 |
| GSEL-LFCS_40-40 | 0.20 |
| GSEL-GAPS_40-40 | 0.17 |
| GSEL-PEG:6_40-40 | 0.16 |
| GSEL-PHYT-PVSA_40-80-80 | 0.16 |
| GSEL-CACL_40-40 | 0.15 |
| GSEL-CHLORG_40_40 | 0.15 |
| GSEL-EPE:2_40-40 | 0.14 |
| GSEL-DADMAC_40-40 | 0.13 |
| GSEL-CCO:75n_40-40 | 0.13 |
| GSEL-PHYT-VB9_40-80-20 | 0.12 |
| GSEL-MAIS_40-40 | 0.12 |
| GSEL-PHYT-C10_80-40-40 | 0.11 |
| GSEL-C10_40-20 | 0.11 |
| GSEL-HDE_40-40 | 0.09 |
| GSEL-NHCO_40-20 | 0.09 |
| GSEL-LABS_40-40 | 0.09 |
| GSEL-PPEG:1_40-40 | 0.08 |
| GSEL-ZNO:R10n_40-40 | 0.07 |
| GSEL-TRHP_40-40 | 0.05 |
| GSEL-PEI:L4_40-40 | 0.00 |
| GSEL-PEI:B25_40-40 | 0.00 |
| GSEL-SPMD_40-40 | 0.00 |
| GSEL-PEG:6_40_40 | 0.00 |
| GSEL-DENDOH4_40-40 | 0.00 |
| GSEL-POXZ:5_40-40 | 0.00 |
| GSEL-DOC_40-40 | 0.00 |
| GSEL-GERAN_40-40 | 0.00 |
| GSEL_40 | 0.00 |
| GSEL-MAND_40-40 | 0.00 |
| GSEL-MALI_40-40 | 0.00 |

TABLE E4
Percent permeability of Tirzepatide from 96 well plate screen

| Formulation ID | Pm (%) |
|---|---|
| GSEL-GCDCA-EDTA-C12E9_40-50-50-50 | 13.21685 |
| GSEL-GCDCA-EDTA-EMUB25_40-50-50-50 | 12.59055 |
| GSEL-CHA-C12E9_40-80-80 | 12.45935 |
| GSEL-GCDCA-EDTA-C12E9_40-50-50-25 | 12.4133 |
| GSEL-GCDCA-EDTA-EMUB25_40-50-25-25 | 12.1169 |
| GSEL-GCDCA-EDTA-C12E9_40-50-25-50 | 11.99776 |
| GSEL-GCDCA_40-50 | 11.81723 |
| GSEL-GCDCA-EDTA_40-50-50 | 11.62497 |
| GSEL-GCDCA-EMUB25_40-80-80 | 11.5221 |
| GSEL-GCDCA-EDTA_40-50-25 | 11.18264 |
| GSEL-GCDCA-EMUB25_40-50-50 | 10.76724 |
| GSEL-GCDCA-EDTA-C12E9_40-50-25-25 | 10.34603 |
| GSEL-CHA-EDTA-C12E9_40-50-50-50 | 9.928231 |
| GSEL-GCDCA-EDTA-EMUB25_40-50-25-50 | 9.768129 |
| GSEL-GCDCA-EDTA-EMUB25_40-50-50-25 | 9.728455 |
| GSEL-CHA-C12E9_40-50-50 | 9.576745 |
| GSEL-CHA-EDTA-C12E9_40-50-50-25 | 9.305622 |
| GSEL-GCDCA-EDTA-C12E9_40-25-50-25 | 8.743742 |
| GSEL-GCDCA-C12E9_40-50-25 | 8.714039 |
| GSEL-GCDCA_40-80 | 8.570564 |
| GSEL-CHA_40-50 | 8.498895 |
| GSEL-CHA-EDTA_40-50-50 | 8.1175 |
| GSEL-CHA_40-80 | 8.014459 |
| GSEL-CHA-EDTA_40-50-25 | 7.879693 |
| GSEL-CHA-EDTA-C12E9_40-50-25-25 | 7.692996 |
| GSEL-GCDCA-EMUB25_40-5025 | 7.631478 |
| GSEL-GCDCA-EDTA-C12E9_40-25-50-50 | 7.438014 |
| GSEL-CHA-BO20_40-50-50 | 7.31578 |
| GSEL-GCDCA-EDTA-C12E9_40-25-25-50 | 7.098189 |
| GSEL-GCDCA-BO20_40-50-50 | 7.027074 |
| GSEL-CHA-EDTA-C12E9_40-50-25-50 | 6.931034 |
| GSEL-GCDCA-EDTA_40-25-50 | 6.877168 |
| GSEL-CHA-BC2_40-50-50 | 6.658912 |
| GSEL-GCDCA-C12E9_40-25-25 | 6.626639 |
| GSEL-TDCA-EMUB25_40-50-50 | 6.433943 |
| GSEL-TDCA-SPAN:20_40-50-50 | 6.322047 |
| GSEL-GCDCA-C12E9_40-50-50 | 6.301421 |
| GSEL-GCDCA-EDTA0C12E9_40-25-25-25 | 6.269613 |
| GSEL-GCDCA-EDTA_40-25-25 | 6.230204 |
| GSEL-GCDCA-EDTA-EMUB25_40-25-50-25 | 5.679205 |
| GSEL-GCDCA-BC10_40-50-50 | 5.106142 |
| GSEL-CHA-BC10_40-50-50 | 4.964145 |
| GSEL-GCDCA-EDTA-EMUB25_40-25-50-50 | 4.901142 |
| GSEL-CHA-C12E9_40-50-25 | 4.88668 |
| GSEL-CHA-EDTA_40-25-50 | 4.654308 |
| GSEL-GCDCA_40-25 | 4.560753 |
| GSEL-CHA-SPAN:20_40-50-50 | 4.549617 |
| GSEL-CHA-EDTA-C12E9_40-25-50-50 | 4.389331 |
| GSEL-GCDCA-EDTA-EMUB25_40-25-25-25 | 4.368537 |
| GSEL-TDCA-C12E9_40-50-50 | 4.350758 |
| GSEL-GCDCA-SPAN:20_40-50-50 | 4.279079 |
| GSEL-CHA-EDTA-C12E9_40-25-50-25 | 4.101758 |
| GSEL-C12E9-NHCO_40-50-50 | 4.028207 |
| GSEL-GCDCA-BC2_40-50-50 | 4.013314 |
| GSEL-GCDCA-EMUB25_40-25-50 | 4.013165 |
| GSEL-GCDCA-EDTA-EMUB25_40-25-25-50 | 3.993747 |
| GSEL-CHA-EDTA-C12E9_40-25-25-50 | 3.261828 |
| GSEL-GCDCA-C12E9_40-25-50 | 3.221055 |
| GSEL-C12E9-C10_40-50-50 | 3.144402 |
| GSEL-CHA-EDTA_40-25-25 | 2.937444 |
| GSEL-CHA-EDTA-C12E9_40-25-25-25 | 2.923489 |
| GSEL-EMUB25-NHCO_40-50-50 | 2.731638 |
| GSEL-EMUB25-C10_40-50-50 | 2.068201 |
| GSEL-GCDCA-EMUB25_40-25-25 | 2.04799 |
| GSEL-CHA-C12E9_40-25-25 | 1.901556 |
| GSEL-CHA_40-25 | 1.701084 |
| GSEL-EDTA-EMUB25_40-50-50 | 1.66328 |
| GSEL-EDTA_40-50 | 1.618043 |
| GSEL-CHA-C12E9_40-25-50 | 1.361133 |
| GSEL-EDTA-C12E9_40-50-50 | 1.323082 |
| GSEL-EDTA-EMUB25_40-25-25 | 1.141762 |
| GSEL-BO20-C10_40-50-50 | 1.03774 |
| GSEL-EDTA-BO20_40-50-50 | 0.916825 |
| GSEL-EMUB25_40-50 | 0.915475 |
| GSEL-EDTA-BC10_40-50-50 | 0.903267 |
| GSEL-EDTA-C12E9_40-50-25 | 0.847212 |
| GSEL-EDTA-EMUB25_40-25-50 | 0.827036 |
| GSEL-EDTA-C12E9_40-25-50 | 0.662824 |
| GSEL-BC10-C10_40-50-50 | 0.657969 |
| GSEL-EDTA-EMUB25_40-50-25 | 0.63902 |
| GSEL-EDTA_40-50 | 0.602215 |
| GSEL-BO20-NHCO_40-50-50 | 0.599431 |
| GSEL-EMUB25_40-25 | 0.561224 |
| GSEL-BC10-NHCO_40-50-50 | 0.510281 |
| GSEL-EDTA-C12E9_40-25-25 | 0.411308 |
| GSEL-C12E9_40-50 | 0.290028 |
| GSEL-C12E9_40-25 | 0.229208 |
| TRIS_50 | 0.106264 |
| GSEL-CHA-C12E9_40-80-80 | 19.22526 |
| GSEL-GCDCA-EDTA-C12E9_40-50-25-50 | 17.62457 |
| GSEL-GCDCA-EDTA-C12E9_40-50-50-25 | 16.53654 |
| GSEL-GCDCA-EDTA_40-50-50 | 15.9349 |
| GSEL-CHA-EDTA-C12E9_40-50-50-25 | 15.30168 |
| GSEL-GCDCA-EDTA-C12E9_40-50-50-50 | 14.07901 |
| GSEL-GCDCA-EDTA-EMUB25_40-50-25-25 | 13.92397 |
| GSEL-CHA_40-80 | 13.89015 |
| GSEL-CHA-EDTA-C12E9_40-50-50-50 | 13.19633 |
| GSEL-CHA-EDTA_40-50-50 | 13.12674 |
| GSEL-GCDCA-EDTA-C12E9_40-50-25-25 | 13.07542 |
| GSEL-CHA-C12E9_40-50-50 | 13.0562 |
| GSEL-GCDCA-EMUB25_40-80-80 | 12.96546 |
| GSEL-CHA-EDTA-C12E9_40-50-25-50 | 12.88981 |
| GSEL-GCDCA-BC2_40-50-50 | 12.68589 |
| GSEL-GCDCA-EMUB25_40-50-50 | 12.52083 |
| GSEL-GCDCA-EDTA-EMUB25_40-50-50-25 | 12.49913 |
| GSEL-GCDCA-EDTA-C12E9_40-25-50-50 | 12.37742 |
| GSEL-GCDCA-EDTA_40-50-25 | 12.36603 |
| GSEL-GCDCA_40-50 | 12.27499 |
| GSEL-GCDCA-EDTA-EMUB25_40-50-50-50 | 12.1432 |
| GSEL-GCDCA-EDTA_40-25-50 | 11.90357 |
| GSEL-GCDCA-EDTA-EMUM25_40-50-25-50 | 11.51363 |
| GSEL-GCDCA-C12E9_40-50-25 | 11.31829 |
| GSEL-GCDCA-EDTA-C12E9_40-25-25-25 | 11.22548 |
| GSEL-CHA-BO20_40-50-50 | 11.14817 |
| GSEL-CHA-BC2_40-50-50 | 11.13578 |
| GSEL-GCDCA-EDTA-C12E9_40-25-50-25 | 10.79234 |
| GSEL-CHA-EDTA_40-50-25 | 10.7526 |
| GSEL-CHA-BC10_40-50-50 | 10.47163 |
| GSEL-GCDCA_40-80 | 10.45806 |
| GSEL-GCDCA-EDTA-C12E9_40-25-25-50 | 10.42155 |
| GSEL-GCDCA-BO20_40-50-50 | 10.12387 |
| GSEL-GCDCA-EDTA_40-25-25 | 10.0068 |
| GSEL-CHA_40-50 | 9.98987 |
| GSEL-TDCA-SPAN:20_40-50-50 | 9.928339 |
| GSEL-GCDCA-BC10_40-50-50 | 9.809394 |
| GSEL-GCDCA-C12E9_40-25-50 | 9.706262 |
| GSEL-GCDCA-EMUB25_40-50-25 | 9.581153 |
| GSEL-CHA-EDTA-C12E9_40-50-25-25 | 9.439793 |
| GSEL-TDCA-EMUB25_40-50-50 | 8.910731 |
| GSEL-GCDCA-EDTA-EMUB25_40-25-50-50 | 8.644788 |
| GSEL-CHA-C12E9_40-50-25 | 8.49035 |
| GSEL-GCDCA-C12E9_40-25-25 | 8.463342 |
| GSEL-GCDCA-C12E9_40-50-50 | 7.951391 |
| GSEL-GCDCA-EDTA-EMUB25_40-25-25-25 | 7.7532929 |
| GSEL-CHA-EDTA-C12E9_40-25-50-25 | 7.75124 |
| GSEL-CHA-EDTA_40-25-50 | 7.678719 |
| GSEL-CHA-EDTA-C12E9_40-25-25-50 | 7.61487 |
| GSEL-GCDCA-EDTA-EMUB25_40-25-50-25 | 7.500642 |
| GSEL-GCDCA_40-25 | 6.949256 |
| GSEL-GCDCA-SPAN:20_40-50-50 | 6.769577 |
| GSEL-GCDCA-EDTA-EMUB25_40-25-25-50 | 6.765907 |
| GSEL-CHA-EDTA-C12E9_40-25-50-50 | 6.474434 |
| GSEL-GCDCA-EMUB25_40-25-50 | 6.341691 |
| GSEL-CHA-EDTA-C12E9_40-25-25-25 | 5.905946 |
| GSEL-C12E9-C10_40-50-50 | 5.816953 |
| GSEL-CHA-SPAN:20_40-50-50 | 5.737598 |
| GSEL-GCDCA-EMUB25_40-25-25 | 5.731883 |
| GSEL-EMUB25-C10_40-50-50 | 5.665801 |
| GSEL-CHA-EDTA_40-25-25 | 5.61438 |
| GSEL-C12E9-NHCO_40-50-50 | 5.446275 |
| GSEL-TDCA-C12E9_40-50-50 | 4.722283 |
| GSEL-EDTA-C12E9_40-50-50 | 4.712769 |
| GSEL-EMUB25-NHCO_40-50-50 | 4.608281 |
| GSEL-CHA-C12E9_40-25-25 | 4.296526 |
| GSEL-CHA-C12E9_40-25-50 | 4.264092 |
| GSEL-EDTA-EMUB25_40-50-50 | 4.25431 |
| GSEL-EDTA-C12E9_40-25-50 | 3.835951 |
| GSEL-BC10-C10_40-50-50 | 3.70159 |
| GSEL-EDTA_40-50 | 3.448007 |
| GSEL-CHA_40-25 | 3.373988 |
| GSEL-EDTA_40-50 | 3.218015 |
| GSEL-C12E9_40-50 | 3.120361 |
| GSEL-EDTA-C12E9_40-50-25 | 3.107643 |
| GSEL-BO20-C10_40-50-50 | 2.891119 |
| GSEL-EDTA-C12E9_40-25-25 | 2.4244 |
| GSEL-EDTA-EMUB25_40-25-50 | 2.357791 |
| GSEL-EDTA-BO20_40-50-50 | 2.33576 |
| GSEL-EDTA-EMUB25_40-50-25 | 2.202875 |
| GSEL-EMUB25_40-50 | 2.198214 |
| GSEL-EMUB25_40-25 | 2.124683 |
| GSEL-EDTA-EMUB25_40-25-25 | 1.916476 |
| GSEL-C12E9_40-25 | 1.901596 |
| GSEL-BO20-NHCO_40-50-50 | 1.860849 |
| GSEL-BC10-NHCO_40-50-50 | 1.837744 |
| GSEL-EDTA-BC10_40-50-50 | 1.456057 |
| Tris_50 | 1.206198 |

TABLE E5
Mean permeability data of Tirzepatide
from 96 well plate screen

| Formulation ID | Pm |
|---|---|
| GSEL-OX-SNAC_40-80-80 | 7.120595 |
| GSEL-OX-PHYT_40-80-40 | 6.806515 |
| GSEL-C08-OX_40-40-80 | 5.808677 |
| GSEL-OX-VB9_40-80-20 | 5.733973 |
| GSEL-OX-SNAC_80-40-80 | 5.728415 |
| GSEL-OX_40-40 | 5.618745 |
| GSEL-OX-PVSA_40-80-80 | 4.966895 |
| GSEL-C10-OX_80-40-80 | 4.392149 |
| GSEL-C10-OX_40-40-80 | 4.202414 |
| GSEL-CHAPS_40-40 | 4.177833 |
| GSEL-NHCO-OX_40-40-80 | 4.04042 |
| GSEL-C10-OX_40-80-80 | 3.406085 |
| GSEL-CHA_40-40 | 3.255406 |
| GSEL-GCDCA_40-40 | 2.337877 |
| GSEL-HPBCD-OX_40-80-80 | 2.313999 |
| GSEL-OX_40-80 | 2.209829 |
| GSEL-GCA_40-40 | 2.007605 |
| GSEL-NHCO_40-40 | 1.699936 |
| GSEL-POXZ:5_40-40 | 1.630138 |
| GSEL-PHYT_40-40 | 1.5604 |
| GSEL-HPBCD-PHYT_40-80-40 | 1.526263 |
| GSEL-VB9_40-40 | 1.405658 |
| GSEL-PVSA_40-40 | 1.116028 |
| GSEL-C10_40-40 | 0.998059 |
| GSEL-C10_40-80 | 0.959741 |
| GSEL-CITR_40-40 | 0.908472 |
| GSEL-C10-PVSA_40-80-80 | 0.834144 |
| GSEL-PEG:1_40-40 | 0.807619 |
| GSEL-CHLORG_40-40 | 0.767041 |
| GSEL-SBECD_40-40 | 0.576162 |
| GSEL-IAA_40-40 | 0.573574 |
| GSEL-EDTA_40-40 | 0.5367 |
| GSEL-C10-HPBCD_40-40-80 | 0.512985 |
| GSEL-KCL_40-40 | 0.470564 |
| GSEL-HPGCD_40-40 | 0.463945 |
| GSEL-C10-VB9_40-80-20 | 0.447494 |
| GSEL-C08_40-40 | 0.438409 |
| GSEL-C10-C08_40-80-40 | 0.393746 |
| GSEL-HCO3_40-40 | 0.388316 |
| GSEL-PVA:30_40-40 | 0.371791 |
| GSEL-C10-C08_40-40-40 | 0.320874 |
| GSEL-C10-PHYT_40-40-40 | 0.320217 |
| GSEL-C10-PHYT_40-80-40 | 0.312469 |
| GSEL-C10-HPBCD_40-80-80 | 0.296566 |
| GSEL-SHB_40-40 | 0.286986 |
| GSEL-HPBCD_40-40 | 0.282702 |
| GSEL-C10-SNAC_40-80-80 | 0.272402 |
| GSEL-PHYT-VB9_40-40-20 | 0.270446 |
| GSEL-DENDOH4_40-40 | 0.264641 |
| GSEL-C10-SNAC_40-40-80 | 0.261132 |
| GSEL-SDS_40-40 | 0.24696 |
| GSEL-VB9_40-20 | 0.230579 |
| GSEL-C10-PVSA_40-40-80 | 0.220385 |
| GSEL-SPMD_40-40 | 0.203597 |
| GSEL-C10-VB9_40-40-20 | 0.176163 |
| GSEL-PHYT-PVSA_40-40-80 | 0.173233 |
| GSEL-DADMAC_40-40 | 0.155933 |
| GSEL-PVP:10_40-40 | 0.146657 |
| GSEL-PHYT-SNAC_40-40-80 | 0.143743 |
| GSEL-SNAC_40-40 | 0.137929 |
| GSEL-CACL_40-40 | 0.129819 |
| GSEL-PEG:6_40-40 | 0.12388 |
| GSEL-CINA_40-40 | 0.110599 |
| GSEL-DGLC_40-40 | 0.109028 |
| GSEL-HPBCD_40-80 | 0.094392 |
| GEL-PEI:B25_40-40 | 0.092415 |
| GSEL-HAP:5u_40-40 | 0.091132 |
| GSEL-EPE:2_40-40 | 0.076187 |
| GSEL-DENDAC2.5_40-40 | 0.056057 |
| GSEL-ZNO:R10n_40-40 | 0.053071 |
| GSEL-40 | 0.020832 |
| GSEL-PEI:L4_40-40 | 0 |
| GSEL-PEP:2_40-40 | 0 |
| GSEL-CCO:75n_40-40 | 0 |
| GSEL-GAPS_40-40 | 0 |
| GSEL-LABS_40-40 | 0 |
| GSEL-LFCS_40-40 | 0 |
| GSEL-DOC_40-40 | 0 |
| GSEL-GERAN_40-40 | 0 |
| OZ_80 | 0 |
| GSEL-PVSA_40-80 | 0 |
| GSEL-SNAC_40-80 | 0 |
| GSEL-C08-PHYT_40-40-40 | 0 |

TABLE E6
Permeability data of tirzepatide delivered by
GSELs including ammonium carbonate with
GCA or ammonium carbonate with CHA

| Formulation | Pm (%) |
|---|---|
| GSEL-GCA-NHCO_40-120-80 | 88.28 |
| GSEL-GCA-NHCO_40-80-80 | 84.60 |
| GSEL-GCA-NHCO_40-40-80 | 63.10 |
| GSEL-GCA-NHCO_40-120-40 | 34.94 |
| GSEL-GCA-NHCO_40-40-40 | 24.82 |
| GSEL-CHA-NHCO_40-80-40 | 21.41 |
| GSEL-GCA-NHCO_40-30-90 | 18.49 |
| GSEL-GCA-NHCO_40-80-40 | 17.42 |
| GSEL-GCA-NHCO_40-120-20 | 16.77 |
| GSEL-GCA-NHCO_40-60-60 | 16.08 |
| GSEL-GCA-NHCO_40-80-20 | 15.75 |
| GSEL-GCA-NHCO_40-90-30 | 15.41 |
| GSEL-NHCO_40-120 | 13.71 |
| GSEL-GCA-NHCO_40-60-20 | 9.01 |
| GSEL-GCA-NHCO_40-20-60 | 5.78 |
| GSEL-NHCO_40-80 | 5.38 |
| GSEL-GCA-NHCO_40-40-20 | 4.49 |
| GSEL-GCA-NHCO_40-30-10 | 3.79 |
| GSEL-GCA-NHCO_40-10-30 | 2.78 |
| GSEL-GCA-NHCO_40-20-20 | 1.32 |
| GSEL-NHCO_40-40 | 1.26 |
| NHCO_40 | 0.47 |
| GSEL-NHCO_40-20 | 0.09 |
| GSEL_40 | 0.00 |

### Example 2A

### Evaluation of Permeation Enhancers—Validation of Permeation Enhancer Combinations

Franz cells were used for testing different permeation enhancers on ex vivo tissue from the small intestine, such as porcine duodenal tissue (or colon tissue where indicated). Water pumps were attached to the Franz cell rack to maintain each Franz cell at 37° C. The racks were loaded with Franz cells, and each cell was filled with 5 mL of phosphate buffered saline. Tissue was cut into ˜3.2×3.2 cm squares and any large clumps of mucus or excrement was removed before mounting onto the Franz cells. Next, gastrointestinal synthetic epithelial lining (GSEL) solutions were prepared by first weighing out dopamine HCl, polydopamine, and a permeation enhancer into a vial. The chosen active pharmaceutical ingredient (API), Tris buffer (50 mM, pH=8.5), and hydrogen peroxide (1M) were then added to the vial, which was then vortexed until all solids were dispersed (brief sonication was used if necessary). 250 μL of the final mixture was then added to the donor compartment of each Franz cell. Samples from the receiving compartment was periodically collected from 0 to 24 hours and the concentration of API was measured. At each sampling point, 200 μL of phosphate buffered saline was added to replace the removed volume (200 μL).

The same experiment was carried out, except without using a permeation enhancer in the GSEL solution. The amount of API detected in the receiving compartment for GSELS with permeation enhancer was compared to the amount of API detected in the receiving compartment for GSELs without permeation enhancer at each time point to determine the increase in permeation provided by the permeation enhancer.

TABLE E7
Percent mean permeability of semaglutide
delivered by GSELs including bile salt and
ammonium carbonate combinations (Franz cell data)

| Formulation | Pm (%) |
|---|---|
| GSEL-CHA-NHCO_40-80-40 | 29.57 |
| GSEL-CHA-SNAC_80-80-80 | 27.00 |
| GSEL-CHA-NHCO_40-40-40 | 24.00 |
| GSEL-CHA_40-160 | 23.14 |
| GSEL-GCDCA_40-80 | 17.50 |
| GSEL-CHA_40-80 | 15.77 |
| GSEL-GCDCA_40-40 | 14.00 |
| GSEL-CHA_40-40 | 8.95 |
| GSEL-GCA_40-40 | 8.55 |
| GSEL-C12_40-40 | 8.33 |
| GSEL-OX_40-40 | 7.00 |
| GSEL-SPAN_40-40 | 6.43 |
| GSEL-C10_40-40 | 3.40 |
| GSEL-NHCO_40-40 | 3.38 |
| GSEL-PhyticA_40-40 | 1.29 |
| GSEL-DCA_40-40 | 1.14 |
| GSEL_40 | 0.86 |

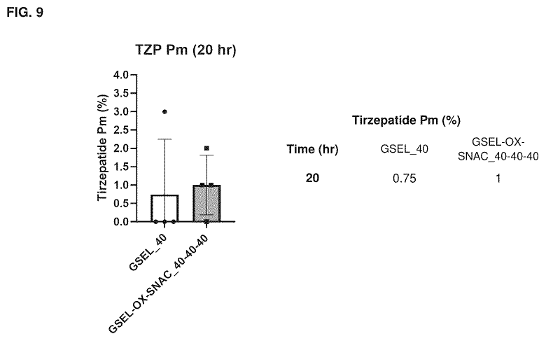

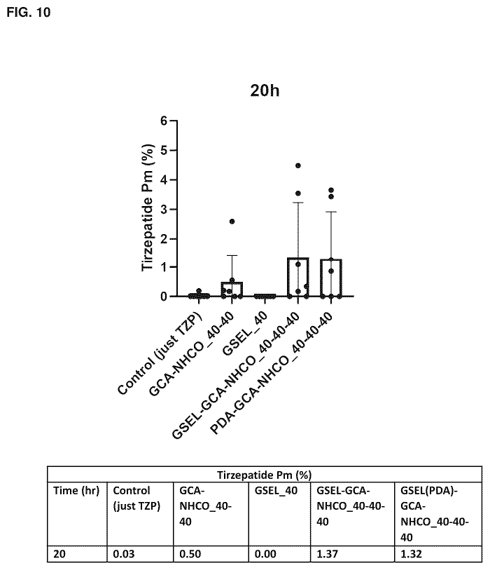

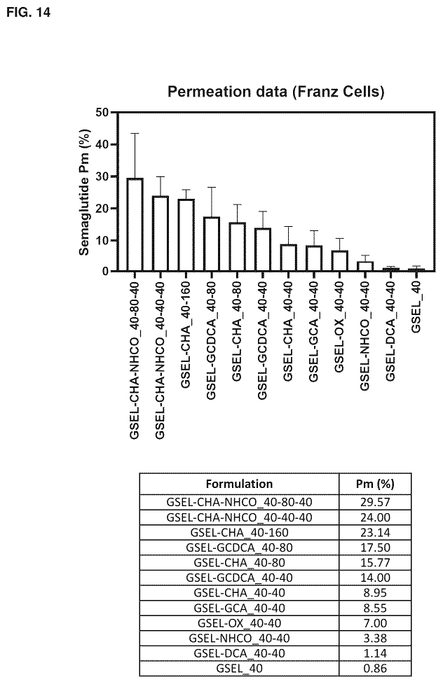

Results: Excipients identified in the 96 well screen were further evaluated for efficacy in Franz cells. API (such as semaglutide and tirzepatide) delivery by liquid and powder formulations of GSEL was measured (FIG. 9, FIG. 10, FIG. 14 and Table E7). Permeability was significantly enhanced for some of the combinations of excipients when the API was delivered by liquid formulation compared to a powder formulation.

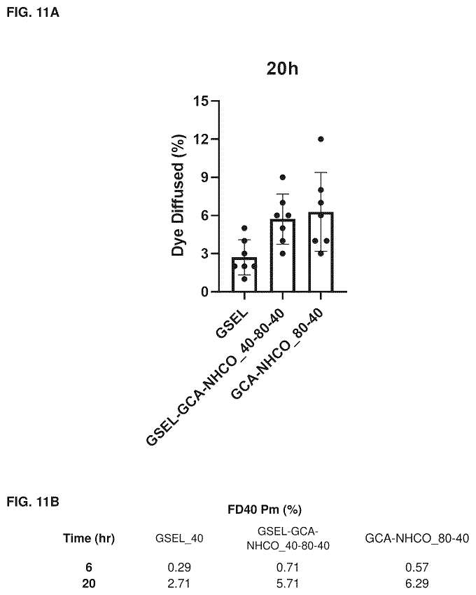

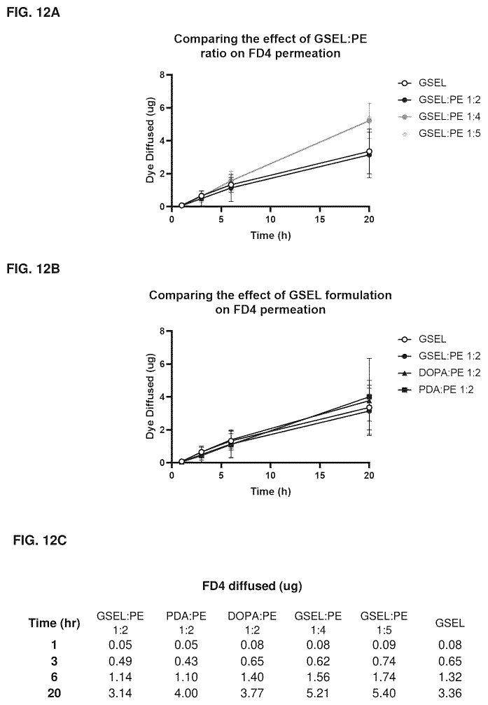

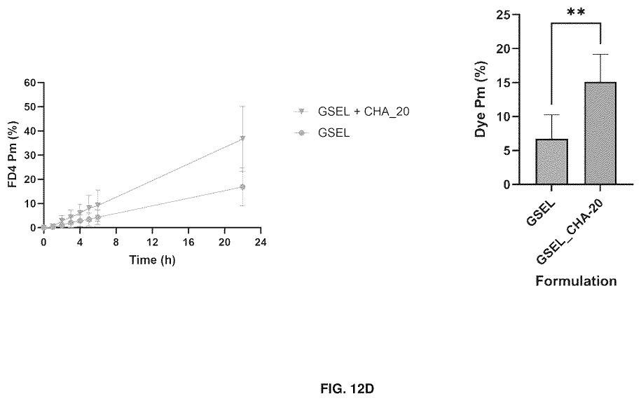

FITC-Dextran molecules were also tested to confirm that pharmaceutical ingredients of varying sizes could be delivered by GSEL. FITC-Dextran-4K (FD4) is a molecule with an approximate molecular weight of 4 kilodaltons. The total amount of FD4 that permeated increased as the ratio of excipient (GCA (40 mg/mL) and NHCO (40 mg/mL) increased relative to GSEL, but the amount of FD4 permeated over time remained similar, irrelevant of GSEL formulation (FIG. 12A, FIG. 12B, FIG. 12C). Permeation was observed to occur at a linear rate (FIG. 12C, FIG. 12D). The permeation enhancers (PE) used were a 1:1 combination of sodium glycocholate (GCA) and ammonium carbonate (NHCO), at 40 mg/ml each, i.e. GCA−NHCO_40-40.

FITC-Dextran-40K (FD40) is a molecule with an approximate molecular weight of 40 kilodaltons. Excipient combinations were tested by assessing FD40 permeability across the GSEL (FIG. 11A, FIG. 11B). Permeation of the API using formulations that included GSEL, GCA and NHCO were comparable to GCA and NHCO alone at 6 and 20 hours, suggesting that the 40 mg/mL GSEL did not inhibit the efficacy of GCA and NHCO as general excipients (FIG. 11A, FIG. 11B).

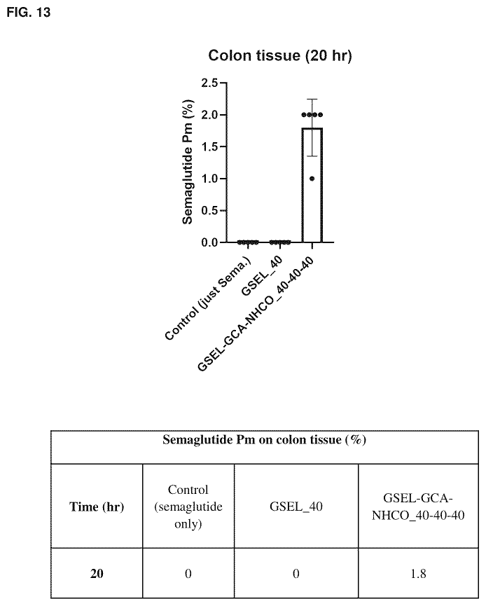

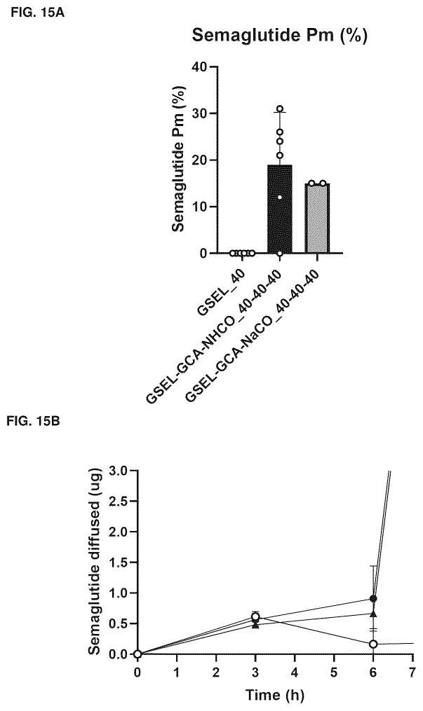

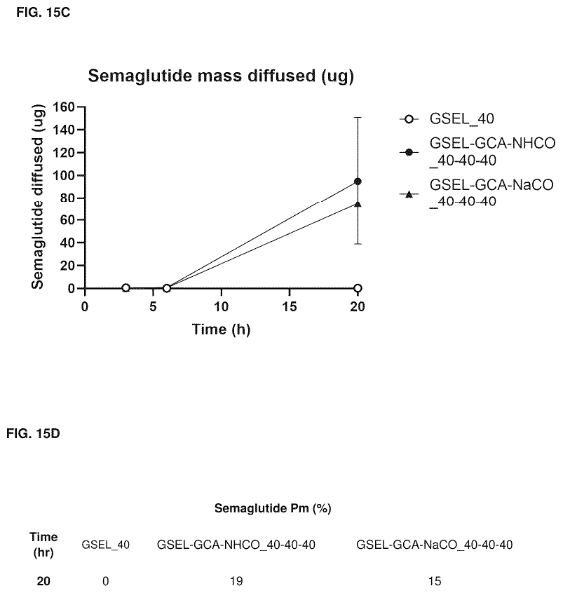

To confirm that the excipient-GSEL combination was effective across multiple tissue types, the assay was also performed using colon tissue. The total amount of semaglutide permeated was significantly enhanced by the addition of NHCO and GCA to the GSEL (FIG. 13, FIG. 14, FIG. 15, FIG. 16). Rate of API permeation was low in the initial six hours, but then increased linearly six hours post delivery (FIG. 15B, FIG. 15C).

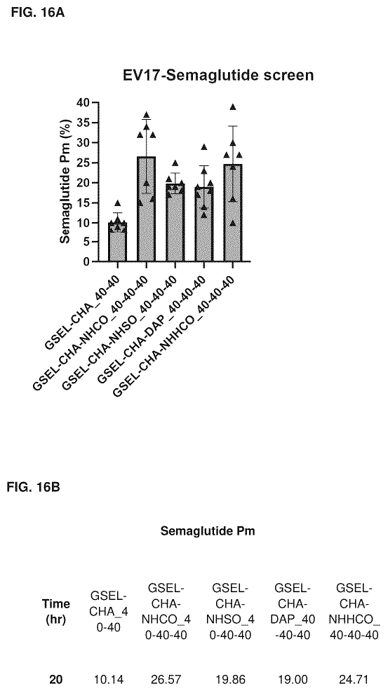

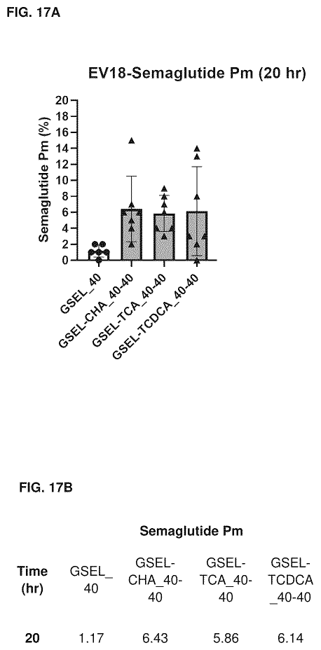

Other excipient combinations were also confirmed by Franz cell experiments (FIG. 17, FIG. 18, FIG. 19). Permeation of semaglutide over duodenal tissue using formulations that included GSEL, GCA and NHCO was observed to be greater compared to GCA and NHCO alone at 20 hours, suggesting that the 40 mg/mL GSEL did not inhibit the efficacy of GCA and NHCO as general excipients (FIG. 19A, FIG. 19B). The data also suggests that certain combinations of excipients enhanced the permeation of the API through the GSEL.

### Example 2B

### Evaluation of Permeation Enhancer Formulations

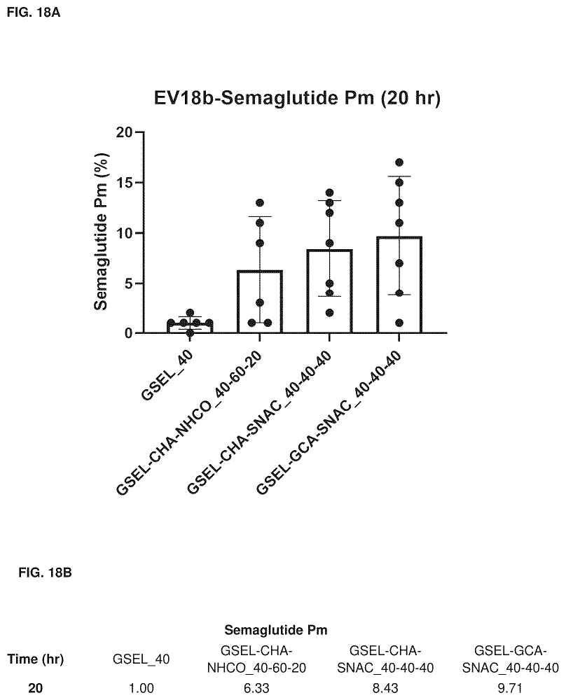

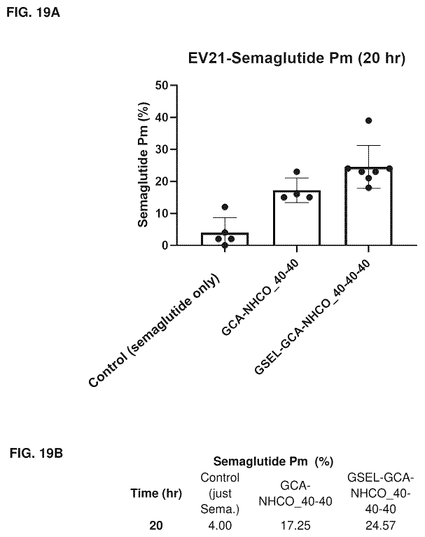

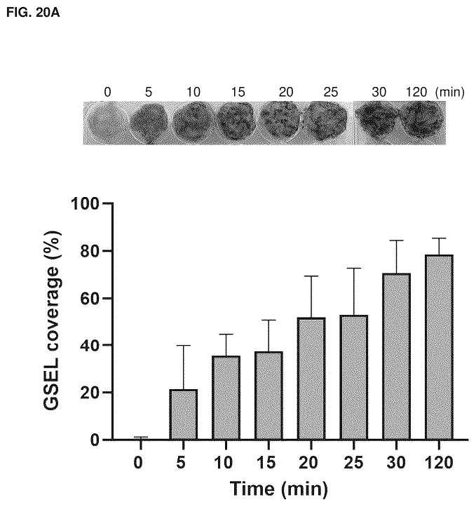

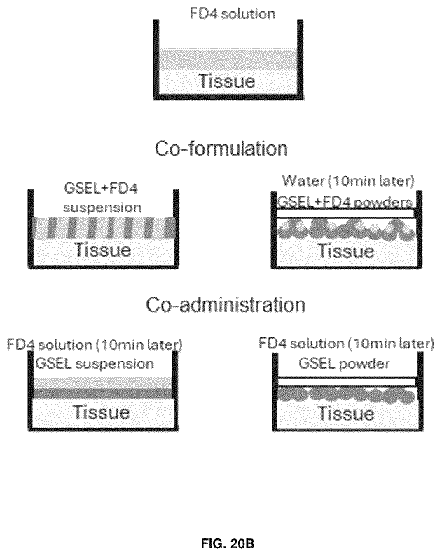

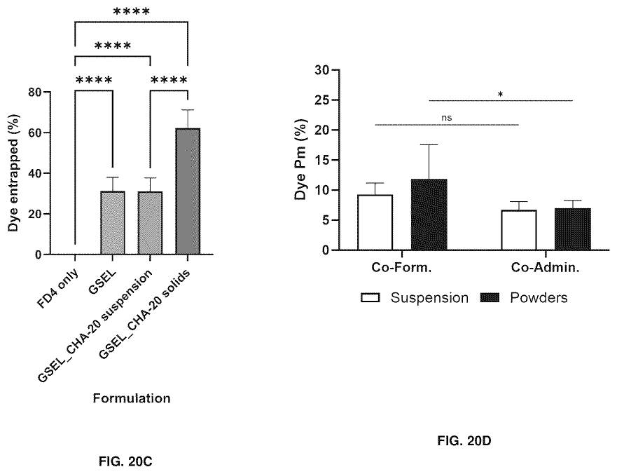

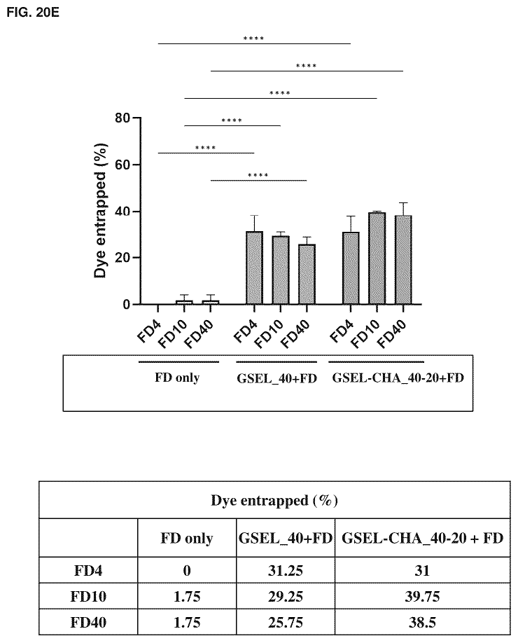

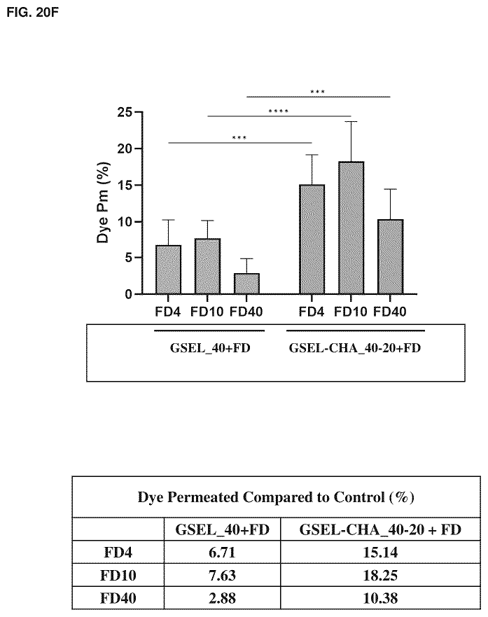

FITC-dextran 4 kDa (FD4), 10 kDa (FD10) and 40 kDa (FD40) were used as model macromolecules to validate the ability of GSEL to entrap macromolecules, and assess GSEL permeability through ex vivo tissue when integrated with permeation enhancers. To determine the rate of GSEL formation, the GSEL components were dispensed on tissue and left to react for different durations. These experiments showed that GSEL incubation past 30 minutes did not seem to significantly change coverage (FIG. 20A).

To test colocalization of FD4 on duodenal tissue, liquid (e.g., suspension) formulations were prepared by adding all components into an aliquot of Tris buffer simultaneously, then directly administered to the tissue (FIG. 20B). For solid (e.g., powder) formulations, all powders and Tris salts were added as solids on the tissue surface and water was added 10 minutes later (FIG. 20B, FIG. 20C, FIG. 20D). Afterwards, the supernatants were measured for FD4 content.

Results: GSEL alone was sufficient to entrap dye (FIG. 20C, FIG. 20E). Further colocalization tests using FD4, FD10, and FD40 showed that co-delivery with GSEL suspensions led to 31.0±6.7%, 29.3±1.7%, and 25.8±3.0% entrapment (FIG. 20E), respectively, compared to the <1% entrapped amount of the control groups (FIG. 20E). The addition of a permeation enhancer, sodium cholate (CHA) led to increased entrapment for FD10 and FD40 (39.8±0.5% and 38.5±5.3, respectively) (FIG. 20E). However, substrates of various sizes were found to permeate the porcine duodenal tissue at a higher percentage when applied with GSEL that comprised a permeation enhancer, versus GSEL with no permeation enhancer (FIG. 20F, FIG. 20G, FIG. 20H). More specifically, the addition of CHA led to 2.25, 2.74, and 3.58-fold increase for FD4, FD10, and FD40 (respectively) after 24 hours (FIG. 20F).

### Example 2C

### Evaluation of Permeation Enhancers Dynamic Assays

The gastrointestinal tract is a dynamic environment. Low entrapment could still permit increased permeability in static conditions, however in a dynamic environment it could translate to low residence time and poorer absorption. On the other hand, high levels of entrapment of semaglutide within the GSEL platform might impair permeability through the tissue because the semaglutide diffusion out of the network is too slow. Thus, it was investigated how changing the ratios among GSEL, permeation enhancer (PE) and semaglutide affected colocalization and permeability. The dynamic assay comprises assaying GSEL delivery on tissue mounted at an angle (FIG. 21).

GCA and NHCO were identified to be highly synergistic with semaglutide and GSEL (FIG. 22A, FIG. 22B), and formulation was modified to improve co-localization and permeation. The formulation (1×GSEL_1×PE) was identified to comprise: 1×GSEL=6.25 mg polydopamine nanoparticles (PDA), 2.45 mg dopamine hydrochloride (DA-HCl), 1×PE=10 mg GCA and 10 mg NHCO.

Using this formulation, a permeability test was executed to test semaglutide co-formulated with GSEL and GCA−NHCO against controls: semaglutide only, semaglutide+SNAC (14:300 by mass based on the Rybelsus tablet), semaglutide+GSEL, and semaglutide+GCA+NHCO (FIG. 22C). In this static Franz cell test, it was evident that GCA-NHCO is a permeation enhancer system that significantly improves permeability to 19.9-fold or 15.5-fold without or with GSEL, respectively (FIG. 22C) In contrast, SNAC did not promote permeation of semaglutide as much (FIG. 22C). Notably, as the Franz tests were static tests, it was hypothesized that GSEL could provide a localized depot to increase residence time at the deposition spot and lead to higher absorption.

Changing the ratio of permeation enhancer to GSEL from 1× to 0.5× or 2× did not improve permeability in a statistically significant manner, indicating that decreasing permeation enhancer concentration by half would not impact the enhancement of permeability (FIG. 22D).

Changing GSEL content revealed that 1×GSEL and 2×GSEL did not seem to have an effect, while 4×GSEL significantly decreased permeability, indicating that increasing the GSEL:PE ratio could have a negative effect on semaglutide release and tissue absorption (FIG. 22E). Under static conditions, increasing GSEL content relative to PE concentration increased semaglutide entrapment (FIG. 22K). However, under dynamic conditions, the GSEL content did not impact entrapment in a statistically significant manner (FIG. 22L). FD4 was used as an exemplary molecule of 4 KDa in size to further verify that increasing the concentration of GSEL relative to permeation enhancer led to greater entrapment in both static and dynamic assays (FIG. 22F, FIG. 22G).

In ex vivo experiments, when permeation enhancer content decreased 10-fold (0.1×PE to GSEL), there was higher colocalization (>60%, FIG. 2H, 22I), in a preliminary dynamic test but permeability decreased to 0.5% (FIG. 22J).

Different ratios of permeation enhancer:semaglutide were examined where increasing amount of semaglutide is co-formulated with 2×GSEL and 1× permeation enhancer. All formulations exhibited similar percent permeability (FIG. 22M).

### Example 3

Gastrointestinal Synthetic Epithelial Lining Formulation with Permeation Enhancer for Administration to Subject: Evaluation in Pig Model

Yorkshire pigs, weighing 40-80 kg each over 7 months and approximately 4-9 months of age were used. Following routine preoperative fast, pigs were anesthetized, intubated, and maintained on isoflurane anesthesia during the procedure. An indwelling marginal ear vein catheter was be inserted for blood collection, which occurred over 1-7 days. Dry GSEL powders containing permeation enhancers and semaglutide were solubilized with Tris buffer immediately before administration. An endoscope guided catheter was placed into the proximal small intestines, where the GSEL suspensions were injected. Blood was taken periodically from the ear vein catheter and the blood was immediately centrifuged down in serum collection tubes. Samples were analyzed via ELISA and LC-MS. Pigs were woken up after 6 hours and monitored until full recovery.

For the following experiment, a ratio of formulation components as presented in Table E8 was used (e.g., 2×GSEL, 6.4 mg/kg GCA, 6.4 mg/kg NHCO, with semaglutide at a final ratio of 20:2.35 permeation enhancer to semaglutide).

TABLE E8
Formulation components

| Component | Amount |
|---|---|
| 1× GSEL | includes 4.55 mg/kg PDA |
| | 1.8 mg/kg DA-HCl |
| 2× GSEL | includes 9.1 mg/kg PDA |
| | 3.6 mg/kg DA-HCl, |
| GCA | 6.4 mg/kg |
| NHCO | 6.4 mg/kg |
| Semaglutide | As indicated in Table E9 |

*AUC = ng*h/mL, Dose = mg/kg, F = Fraction absorbed.

Results: GSEL formulations comprising permeation enhancers were identified which result in semaglutide plasma concentrations significantly greater in the pigs receiving the GSEL formulation compared to the pigs receiving the non-GSEL formulation or the pigs receiving semaglutide alone.

PK curves are presented for intravenous administration of semaglutide alone, versus endoscopic placement of semaglutide (control), versus endoscopic placement of semaglutide delivered with GSEL with permeation enhancers at various formulations, versus endoscopic placement of semaglutide delivered with the permeation enhancers alone as a control (FIG. 23A, FIG. 23B, FIG. 23C). Based on the PK values, curves the area under the curve (AUC) was calculated for each group and the results were plotted for different timepoints at 24, 48, 72 and 168 hours (FIG. 23D, FIG. 23E, FIG. 23F, FIG. 23G). In all cases, the delivery of semaglutide with GSEL and permeation enhancers demonstrated at least a 3-fold increase in semaglutide uptake, as compared to the control (semaglutide administered with permeation enhancers), while delivery of semaglutide without any PEs showed zero absorption for 1.5 mg/kg dose for n=1. This result is likely because of the depot effect that the GSEL provides, increasing the retention time of the macromolecule on the duodenal tissue resulting in enhanced absorption. The absolute bioavailabilities (e.g., F=fraction absorbed) are shown in Table E9, with semaglutide delivered with GSEL/permeation enhancers achieving bioavailabilities ranging from 1.9-3.1%. The GSEL/permeation enhancer formulation was observed to be more effective in comparison to the GSEL alone dose, as shown from the AUC up to 48 hours post administration (Table E9).

TABLE E9
Bioavailability results for all groups tested in vivo.

| | | | *AUC/ | *AUC/ | *AUC/ | *AUC/ | | | | | |
|---|---|---|---|---|---|---|---|---|---|---|---|
| | | | norm. | norm. | norm. | norm. | | | | | |
| | Semaglutide | | dose | dose | dose | dose | F | F | F | F | |
| Group | dose | | (24 h) | (48 h) | (72 h) | (168 h) | (24 h) | (48 h) | (72 h) | (168 h) | n |
| IV/dose (1 mg) | 1 | mg | 174602 | 274533 | 358819 | 656498 | | | | | 2 |
| Control | 1.5 | mg/kg | 0 | 0 | 0 | 0 | 0 | 0 | 0 | | 1 |
| (semaglutide | | | | | | | | | | | |
| only) | | | | | | | | | | | |
| Control (1x | 1.5 | mg/kg | 1771 | 2469 | 2767 | 3232 | 1% | 0.9% | 0.8% | 0.5% | 5 |
| permeation | | | | | | | | | | | |
| enhancer) | | | | | | | | | | | |
| 1x GSEL | 1.5 | mg/kg | 3372 | 6142 | | | 2% | 2% | | | 3 |
| combined with | | | | | | | | | | | |
| 1x permeation | | | | | | | | | | | |
| enhancer | | | | | | | | | | | |
| 2x GSEL | 1.5 | mg/kg | 5327 | 8410 | 9703 | 12164 | 3.1% | 3.1% | 2.7% | 1.9% | 4 |
| combined with | | | | | | | | | | | |
| 1x permeation | | | | | | | | | | | |
| enhancer | | | | | | | | | | | |
| 2xGSEL_1xPE | | | | | | | | | | | |

### Example 4

Gastrointestinal Synthetic Epithelial Lining Formulation with Permeation Enhancer for Administration to Subject: Evaluation in Dog Model

A total of 12 (6 male, 6 female) beagles weighing 9-12 kg and approximately 6-14 months of age are used. Dogs will be trained to swallow capsules/tablets and test articles will be administered orally following an overnight fast. Following dosing, 15 mL of saline will be administered as a post-dose flush using a syringe. Blood samples will be collected via cephalic venipuncture over 48 hours (0, 0.33, 0.66, 1, 2, 4, 6, 8, 24, 48) and then daily over 7 days. Samples will be collected into serum separation tubes. Samples will be analyzed via ELISA and LC-MS. The table below illustrates one potential formulation for GSELs with permeation enhancers for the dog studies.

Component

Semaglutide
Excipient(s) for permeation
enhancement (e.g., permeation
enhancer(s))
Dopamine
Polydopamine
Urea H₂O₂
Tris acid
Tris base
Aerosil (other excipients)

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain changes and modifications will be practiced. Therefore, the description and examples should not be construed as limiting the scope of the invention.

1. A composition for oral administration for forming a polymer in situ in a subject, comprising:
a polymer precursor;
an oxygen source; and
a permeation enhancer that enhances permeation of one or more active pharmaceutical ingredients.

2. The composition of claim 1, further comprising one or more active pharmaceutical ingredients.

3. (canceled)

4. The composition of claim 1, further comprising one or more additional permeation enhancers.

5. The composition of claim 1, wherein the composition comprises an amount of polymer precursor to (polymer precursor, oxygen source, and permeation enhancer) of 40% to 90%.

6. The composition of claim 1, wherein the composition comprises an amount of oxygen source to (polymer precursor, oxygen source, and permeation enhancer) of 1% to 15%.

7-11. (canceled)

12. The composition of claim 2, wherein the one or more active pharmaceutical ingredients is a macromolecule having a molecular weight between about 1 kD and about 160 kD.

13. (canceled)

14. The composition of claim 12, wherein the macromolecule is a polypeptide.

15. The composition of claim 14, wherein the polypeptide has a molecular weight of between about 1 kD and about 10 kD.

16. The composition of claim 14, wherein the polypeptide comprises between about 8 and about 80 amino acids.

17. The composition of claim 14, wherein the polypeptide comprises insulin, semaglutide, a GLP-1 receptor agonist, tirzepatide, liraglutide, desmopressin, octreotide, an analgesic peptide, difelikefalin, H-20, an antibiotic, cyclosporin, vancomycin, lactase, beta galactosidase, exenatide, teriparatide, nafarelin, buserelin, captopril, daptomycin, an antibody, caplacizumab, ozoralizumab, brolucizumab, ranibizumab, bevacizumab, trastuzumab, rituximab, adalimumab, an enzyme, lipase, a protease, phenylalanine hydroxylase, carbamoylphosphate synthetase I, glucose oxidase, or L-asparaginase.

18. The composition of claim 12, wherein the macromolecule is a polynucleotide.

19-28. (canceled)

29. The composition of claim 2, wherein the one or more active pharmaceutical ingredients is a small molecule having a molecular weight of 1 kD or less.

30. The composition of claim 1, wherein the polymer precursor comprises one or both of a monomer and an oligomer precursor to a polymer.

31. (canceled)

32. The composition of claim 30, wherein the monomer is dopamine, levodopa, norepinephrine, methyldopa, levodopa methyl ester, levodopa ethyl ester, or combinations thereof.

33. The composition of claim 1, wherein the oxygen source is a substrate for an endogenous catalyst.

34. The composition of claim 33, wherein the oxygen source is urea hydrogen peroxide or hydrogen peroxide.

35. The composition of claim 1, wherein the composition is in an oral dosage form.

36-38. (canceled)

39. The composition of claim 1, wherein the permeation enhancer is a selected from the group consisting of: an ammonium salt, a carbonate salt, a bicarbonate salt, an endogenous secretion, a bile salt, a bile acid, a mixture of a bile salt and a bile acid, a carnitine, an acyl carnitine, a choline, an aromatic alcohol, a piperazine derivative, a mucoadhesive polymer, a cell penetrating peptide, an amino acid, an ionic liquid, an organic solvent, an anionic surfactant, a chelating agent, a non-ionic surfactant, a non-ionic detergent, a fatty acid, a fatty acid salt, an ethoxylated fatty acid ester, a sugar fatty acid ester, an ethoxylated sugar fatty acid ester, an N-acylated acid, a high molecular weight polymer, a sugar-based surfactant, ammonium carbonate, ammonium sulfate, ammonium citrate, ammonium phosphate, diammonium phosphate, monoammonium phosphate, ammonium bicarbonate, ammonium chloride, ammonium lactate, ammonium acetate, ammonium sulfate, ammonium sulfite, triammonium citrate, ammonium propionate, ammonium sulfamate, sodium carbonate, potassium carbonate, sodium bicarbonate, potassium bicarbonate, cholic acid, glycocholic acid, deoxycholic acid, glychochenodeoxychlic acid, glycodeoxycholic acid, taurodeoxycholic acid, taurocholic acid, bovine bile, chenodeoxycholic acid, taurochenodeoxycholic acid, lithocholic acid, glycolithocholate, glycohyocholate, taurolithocholate, ursodeoxycholic acid, tauroursodeoxycholic acid, glycoursodeoxycholic acid, 12-monokctocholic acid (12-MKC), 7-monoketocholic acid (7-MKC), 7,12-diketocholic acid (7,12-DKC), 3,7,12-triketocholic acid (3,7,12-TKC), 12-monoketodeoxycholic acid (12-MKDC), taurodihydrofusidic acid, 3-[(3-cholamidopropyl)dimethylammonio]-1-propanesulfonate (CHAPS), a salt of cholic acid, a salt of glycocholic acid, a salt of deoxycholic acid, a salt of glychochenodeoxychlic acid, a salt of glycodeoxycholic acid, a salt of taurodeoxycholic acid, a salt of taurocholic acid, a salt of bovine bile, a salt of chenodeoxycholic acid, a salt of taurochenodeoxycholic acid, a salt of lithocholic acid, a salt of glycolithocholate, a salt of glycohyocholate, a salt of taurolithocholate, a salt of ursodeoxycholic acid, a salt of tauroursodeoxycholic acid, a salt of glycoursodeoxycholic acid, a salt of 12-monoketocholic acid, a salt of 7-monokctocholic acid, a salt of 7,12-diketocholic acid, a salt of 3,7,12-triketocholic acid, a salt of 12-monokctodeoxycholic acid, a salt of taurodihydrofusidic acid, a salt of 3-[(3-cholamidopropyl)dimethylammonio]-1-propanesulfonate, sodium cholate, sodium glycocholate, sodium deoxycholate, sodium glychochenodeoxychlate, sodium glycodeoxycholate, sodium taurodeoxycholate, sodium taurocholate, a sodium salt of bovine bile, sodium chenodeoxycholate, sodium taurochenodeoxycholate, sodium lithocholate, sodium glycolithocholate, sodium glycohyocholate, sodium taurolithocholate, sodium ursodeoxycholate, sodium tauroursodeoxycholate, sodium glycoursodeoxycholate, sodium 12-monoketocholate, sodium 7-monoketocholate, sodium 7,12-diketocholate, sodium 3,7,12-triketocholate, sodium 12-monokctodeoxycholate, sodium taurodihydrofusidate, sodium 3-[(3-cholamidopropyl)dimethylammonio]-1-propanesulfonate, lauroylcarnitine, palmitoylcarnitine, palmitoyl carnitine chloride (PCC), lysophosphatidyl choline, benzyl alcohol, phenyl alcohol, phenoxyethanol, propyl gallate, butylated hydroxytoluene, butylated hydroxyanisole, 1-phenylpiperazinc, 1-methyl-4-phenylpiperazine, 1-(4-methylphenyl)piperazine, chitosan, chitosan hydrochloride, trimethylated chitosan chloride, N,N,N-trimethyl chitosan chloride, transportan, penetratin, oligoarginines, polyarginines, oligolysines, polylysines, oligotryptophans, polytryptophans, tryptophan, choline geranate, nicotinic acid, trigonelline, ethanol, 2-propanol, 1-propanol, 2-methyl-2-propanol, dimethyl sulfoxide, ethyl acetate, acetone, sodium cholate, a pharmaceutically acceptable salt of cholic acid, cholic acid, a mixture of cholic acid and a pharmaceutically acceptable salt of cholic acid, sodium dodecyl sulphate, a pharmaceutically acceptable salt of dodecyl hydrogen sulphate, dodecyl hydrogen sulphate, a mixture of dodecyl hydrogen sulphate and sodium dodecyl sulphate, EDTA, EGTA, DTPA, an ethoxylate, an alcohol ethoxylate (C_{X}E_{Y}, where X is the alcohol carbon number and Y is the ethylene oxide number), a medium or long chain fatty acid sugar ester, sodium laurate, a medium or long chain fatty acid sucrose ester, an ethoxylated fatty acid sugar ester, an ethoxylated sorbitan ester, an ethoxylated glyceride, macrogol-8 glyceride, sucrose esters, sucrose laurate, alkyl maltosides, dodecyl maltoside, short chain polyethylene glycol, Brij® series (polyoxyethylene(10) oleyl ether, polyoxyethylene (23) lauryl ether, etc.), polysorbates, polysorbate series PS 20, PS 40, PS 60, PS 65, PS 80, Triton X-100, caprylocaproyl polyoxyl-8 glyceride, poloxamers, polyoxylglycerides, polyethylene monostearate, sucrose monolaurate, n-Tetradecyl β-D-maltopyranoside, sodium caprate, sodium caprylate, nonaethylene glycol monododecyl ether, a fatty acid ester of a monosaccharide, an ethoxylated fatty acid ester of a monosaccharide, a fatty acid ester of sorbitan, a fatty acid ester of glucose, an ethoxylated fatty acid ester of sorbitan, an ethoxylated fatty acid ester of glucose, a fatty acid ester of a disaccharide, an ethoxylated fatty acid ester of a disaccharide, a fatty acid ester of sucrose, a fatty acid ester of maltose, an ethoxylated fatty acid ester of sucrose, an ethoxylated fatty acid ester of maltose, dodecylmaltoside, sodium dodecyl sulfate, nonaethylene glycol monododecyl ether, sodium laurate, sodium nonanoate, sodium undecanoate, sodium undecylenate, sodium oleate, linoleic acid, sucrose monolaurate, acetylsalicylic acid, sodium N-[8-(2-hydroxybenzoyl)amino]caprylate (SNAC), 8-(N-2-hydroxy-5-chloro-benzoyl)-amino-caprylic acid (5-CNAC), 4-[(4-chloro-2-hydroxy-benzoyl)amino]butanoic acid (4-CNAB), N-(10-[2-hydroxybenzoyl]-amino)decanoic acid (SNAD), monosodium N-(4-chlorosalicyloyl)-4-aminobutyrate (5-CNAB), N-[8-(2-hydroxy-4-methoxy)benzoyl]amino caprylic acid (4-MOAC), a polysaccharide, an antibacterial toxin, zonula occludens toxin analogs, viral protein 8 analogs, Clostridium perfringens enterotoxin analogs, chitosan, carboxymethylcellulose, a caprylocaproyl PEG 8 glyceride, dodecyl-β-D-maltopyranoside (DDM), glyceryl monocaproate, urea, sodium docusate, citric acid, pharmaceutically acceptable salts of any of the foregoing acids or bases, alternative pharmaceutically acceptable salts of any of the foregoing salts, the free acids of any of the foregoing acid salts, and the free bases of any of the foregoing base salts.

40-98. (canceled)

99. The composition of claim 1, wherein the permeation enhancer is a bile salt.

100. The composition of claim 99, wherein the bile salt is selected from the group consisting of sodium taurodeoxycholate, sodium taurocholate, sodium cholate, sodium deoxycholate, sodium glycodeoxycholate, sodium glycochenodeoxycholate, sodium glycocholate, sodium chenodeoxycholate, sodium taurochenodeoxycholate, sodium lithocholate, sodium ursodeoxycholate, sodium tauroursodeoxycholate, sodium glycoursodeoxycholate, and mixed sodium taurodihydrofusidate, or an alternative pharmaceutically acceptable salt thereof.

101. The composition of claim 99, wherein the bile salt is sodium cholate, or an alternative pharmaceutically acceptable salt thereof.

102-125. (canceled)

126. A method of forming a polymer coating in the small intestine of a subject, the method comprising administering to a subject the composition of claim 1.

127. A method of forming a polymer coating in the small intestine of a subject, the method comprising orally administering to a subject:
a polymer precursor;
an oxygen source; and
a permeation enhancer that enhances uptake of one or more active pharmaceutical ingredients;
wherein the polymer precursor and the oxygen source contact a catalyst endogenous to the subject and the catalyst polymerizes the polymer precursor.

128. The method of claim 127, further comprising administering an active pharmaceutical ingredient to the subject.

129. The method of claim 127, wherein the polymer precursor, the oxygen source and the permeation enhancer are administered as a single composition.

130. The method of claim 128, wherein the polymer precursor, the oxygen source, the permeation enhancer, and the active pharmaceutical ingredient are administered as a single composition.

131. The composition of claim 14, wherein the polypeptide comprises semaglutide.

132. The composition of claim 14, wherein the polypeptide comprises tirzepatide.

133-134. (canceled)

135. The method of claim 127, wherein the permeation enhancer is a bile salt.

136. The method of claim 127, wherein the permeation enhancer is selected from the group consisting of sodium taurodeoxycholate, sodium taurocholate, sodium cholate, sodium deoxycholate, sodium glycodeoxycholate, sodium glycochenodeoxycholate, sodium glycocholate, sodium chenodeoxycholate, sodium taurochenodeoxycholate, sodium lithocholate, sodium ursodeoxycholate, sodium tauroursodeoxycholate, sodium glycoursodeoxycholate and mixed sodium taurodihydrofusidate, the corresponding acids thereof, an alternative pharmaceutically acceptable salt thereof, and a mixture of a salt and an acid thereof.
